SEQUENCE LISTING (21 sequences)

SEQ ID NO: 1 - DNA, 513 bp, Anabaena sp. (PCC7119)
  variation (43)..(45): wherein aaa is replaced by tta
atgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaa

SEQ ID NO: 2 - protein, 170 aa, Anabaena sp. (PCC7119)
  VARIANT (15)..(15): wherein Lys is replaced by Leu
MSKKIGLFYGTQTGKTESVAEIIRDEFGNDVVTLHDVSQAEVTDLNDYQYLIIGCPTWNIGELQSDWEGLYSELDDVDFNGKLVAYFGTGDQIGYADNFQDAIGILEEKISQRGGKTVGYWSTDGYDFNDSKALRNGKFVGLALDEDNQSDLTDDRIKSWVAQLKSEFGL

SEQ ID NO: 3 - DNA, 162 bp, Pisum sativum
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcata

SEQ ID NO: 4 - protein, 54 aa, Pisum sativum
MAAAVTAAVSLPYSNSTSLPIRTSIVAPERLVFKKVSLNNVSISGRVGTIRALI

SEQ ID NO: 5 - DNA, 675 bp, Artificial sequence
  Coding sequence for fusion protein with transit peptide and flavodoxin
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaa

SEQ ID NO: 6 - protein, 224 aa, Artificial sequence
  Fusion protein with pea transit peptide and anabaena flavodoxin
MAAAVTAAVSLPYSNSTSLPIRTSIVAPERLVFKKVSLNNVSISGRVGTIRALIMSKKIGLFYGTQTGKTESVAEIIRDEFGNDVVTLHDVSQAEVTDLNDYQYLIIGCPTWNIGELQSDWEGLYSELDDVDFNGKLVAYFGTGDQIGYADNFQDAIGILEEKISQRGGKTVGYWSTDGYDFNDSKALRNGKFVGLALDEDNQSDLTDDRIKSWVAQLKSEFGL

SEQ ID NO: 7 - DNA, 1179 bp, Oryza sativa
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacacca

SEQ ID NO: 8 - DNA, 195 bp, Cyanophora paradoxa
atggccttcgtcgcgtctgtccccgtcttcgccaacgcctctggccttaagaccgaggctaaggtctgccagaagcccgcgctgaagaacagcttcttccgcggcgaggaggttacctctcgctcgttcttcgccagccaggctgtgtcggcgaagccggcgaccaccttcgaggtcgacaccaccatccgcgcg

SEQ ID NO: 9 - DNA, 195 bp, Cyanophora paradoxa
atggctttcgttgccagcgtgccagttttcgctaacgcttccggccttaaaactgaagccaaggtgtgccagaagcctgccttgaagaattcatttttcaggggcgaggaagtcacatctagatctttttttgcctcccaagcagtgtccgctaaaccagcaacaaccggcgaggttgatactaccattagggca

SEQ ID NO: 10 - protein, 65 aa, Cyanophora paradoxa
  VARIANT (57)..(57): Gly is replaced by Phe
MAFVASVPVFANASGLKTEAKVCQKPALKNSFFRGEEVTSRSFFASQAVSAKPATTGEVDTTIRA

SEQ ID NO: 11 - protein, 235 aa, Artificial sequence
  Artifiical fusion protein of C.paradoxa transit peptide SEQ ID NO 10 and Anabaena flavodoxin SEQ ID NO 2
MAFVASVPVFANASGLKTEAKVCQKPALKNSFFRGEEVTSRSFFASQAVSAKPATTGEVDTTIRAMSKKIGLFYGTQTGKTESVAEIIRDEFGNDVVTLHDVSQAEVTDLNDYQYLIIGCPTWNIGELQSDWEGLYSELDDVDFNGKLVAYFGTGDQIGYADNFQDAIGILEEKISQRGGKTVGYWSTDGYDFNDSKALRNGKFVGLALDEDNQSDLTDDRIKSWVAQLKSEFGL

SEQ ID NO: 12 - DNA, 2082 bp, Artificial sequence
  artificial expression cassette comprising nucleic acid sequences for the promoter of SEQ ID NO 7, coding the C.paradoxa transit peptide of SEQ ID NO 10 fused to the flavodoxin of SEQ ID NO2 and a tZein terminator
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacaccaatggctttcgttgccagcgtgccagttttcgctaacgcttccggccttaaaactgaagccaaggtgtgccagaagcctgccttgaagaattcatttttcaggggcgaggaagtcacatctagatctttttttgcctcccaagcagtgtccgctaaaccagcaacaaccggcgaggttgatactaccattagggcaatgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaacgatgattgagtaataatgtgtcacgcatcaccatgggtggcagtgtcagtgtgagcaatgacctgaatgaacaattgaaatgaaaagaaaaaaagtactccatctgttccaaattaaaattggttttaaccttttaataggtttatacaataattgatatatgttttctgtatatgtctaatttgttatcatcc

SEQ ID NO: 13 - DNA, 2082 bp, Artificial sequence; identical to SEQ ID NO: 12
  artificial sequence, Anabaena Flavodoxin codon usage optimized for higher plants, encoding the polypeptide of SEQ ID NO 2

SEQ ID NO: 14 - DNA, 675 bp, Artificial sequence
  artifiical sequence fusing the nucleic acid of SEQ ID NO 3 encoding the transit peptide of SEQ ID NO 4 to the codon optimized nculeic acid of SEQ ID NO 13 encoding the flavodoxin of SEQ ID NO 2
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgagcaagaagatcggcctgttttacggcacgcaaaccggcaaaaccgagagcgtcgcggagatcattagggacgagttcgggaacgacgtggtgacactccacgacgtcagccaagcggaggttacggacctgaacgactaccagtacctgatcatcgggtgccccacctggaatataggcgagttgcaatcggactgggagggcttgtactctgagcttgacgacgtggacttcaacgggaagctggtggcttacttcggtacgggtgaccagattggctacgcagacaacttccaggacgccataggcatcctcgaggagaagatcagccagagaggtgggaaaaccgtgggttactggagcactgacggctacgacttcaacgactcgaaggcgttgaggaacggcaagttcgtgggtcttgcgctcgacgaggacaaccagtccgatctcacagacgacaggatcaagtcctgggtggctcagctgaaatccgagttcggcctttag

SEQ ID NO: 15 - DNA, 513 bp, Synechocystis PCC6803
atgacaaaaattggacttttttacggtactcaaaccggcaacactgaaaccattgctgaactgattcaaaaagaaatgggcggcgatagtgtggtcgatatgatggatatatcccaggctgatgttgatgattttaggcaatatagttgcctgattatcggttgtcccacctggaatgtgggggaactccagagtgattgggaaggcttttatgaccaattagacgaaattgattttaatggcaaaaaagtagcctattttggtgctggcgatcaggttggttatgcagataattttcaagacgccatgggcattttagaagaaaaaatcagtggattaggcggtaaaacagtggggttttggcccaccgctggctatgattttgacgaatcaaaagcggtgaaaaatgggaaatttgttggtttagctttggacgaagataatcagccagagttaacagaattaagagtaaagacatgggtaagtgaaattaaaccaattttgcaatcctaa

SEQ ID NO: 16 - protein, 170 aa, Synechocystis PCC6803
MTKIGLFYGTQTGNTETIAELIQKEMGGDSVVDMMDISQADVDDFRQYSCLIIGCPTWNVGELQSDWEGFYDQLDEIDFNGKKVAYFGAGDQVGYADNFQDAMGILEEKISGLGGKTVGFWPTAGYDFDESKAVKNGKFVGLALDEDNQPELTELRVKTWVSEIKPILQS

SEQ ID NO: 17 - DNA, 675 bp, Artificial sequence
  fusion of the nucleic acid of SEQ ID NO 3 encoding the transit peptide of SEQ ID NO 4 and the nucleic acid of SEQ ID NO 15 encoding the flavodoxin of SEQ ID NO 16
atggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgacaaaaattggacttttttacggtactcaaaccggcaacactgaaaccattgctgaactgattcaaaaagaaatgggcggcgatagtgtggtcgatatgatggatatatcccaggctgatgttgatgattttaggcaatatagttgcctgattatcggttgtcccacctggaatgtgggggaactccagagtgattgggaaggcttttatgaccaattagacgaaattgattttaatggcaaaaaagtagcctattttggtgctggcgatcaggttggttatgcagataattttcaagacgccatgggcattttagaagaaaaaatcagtggattaggcggtaaaacagtggggttttggcccaccgctggctatgattttgacgaatcaaaagcggtgaaaaatgggaaatttgttggtttagctttggacgaagataatcagccagagttaacagaattaagagtaaagacatgggtaagtgaaattaaaccaattttgcaatcctaa

SEQ ID NO: 18 - protein, 224 aa, Artificial sequence
  fusion protein of the transit peptide of SEQ ID NO 4 and the flavodoxin of SEQ ID NO 16
MAAAVTAAVSLPYSNSTSLPIRTSIVAPERLVFKKVSLNNVSISGRVGTIRALIMTKIGLFYGTQTGNTETIAELIQKEMGGDSVVDMMDISQADVDDFRQYSCLIIGCPTWNVGELQSDWEGFYDQLDEIDFNGKKVAYFGAGDQVGYADNFQDAMGILEEKISGLGGKTVGFWPTAGYDFDESKAVKNGKFVGLALDEDNQPELTELRVKTWVSEIKPILQS

SEQ ID NO: 19 - DNA, 1907 bp, Artificial sequence
  the promoter of SEQ ID NO 7 combined with the nucleic acid of SEQ ID NO 3 for the transit peptide of SEQ ID NO 4 and the nucleic acid of SEQ ID NO 1 coding for the flavodoxin of SEQ ID NO 2, with a short linker sequence between the promoter & CDS
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacaccaatttaaatcaactagggatatcacaagtttgtacaaaaaagcaggcttaaacaatggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgtcaaagaaaattggtttattctacggtactcaaactggtaaaactgaatcagtagcagaaatcattcgagacgagtttggtaatgatgtggtgacattacacgatgtttcccaggcagaagtaactgacttgaatgattatcaatatttgattattggctgtcctacttggaatattggcgaactgcaaagcgattgggaaggactctattcagaactggatgatgtagattttaatggtaaattggttgcctactttgggactggtgaccaaataggttacgcagataattttcaggatgcgatcggtattttggaagaaaaaatttctcaacgtggtggtaaaactgtcggctattggtcaactgatggatatgattttaatgattccaaggcactaagaaatggcaagtttgtaggactagctcttgatgaagataatcaatctgacttaacagacgatcgcatcaaaagttgggttgctcaattaaagtctgaatttggtttgtaa

SEQ ID NO: 20 - DNA, 1907 bp, Artificial sequence
  the promoter of SEQ ID NO 7 combined with the nucleic acid of SEQ ID NO 3 for the transit peptide of SEQ ID NO 4 and the nucleic acid of SEQ ID NO 13 coding for the flavodoxin of SEQ ID NO 2, with a short linker sequence between the promoter & CDS
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacaccaatttaaatcaactagggatatcacaagtttgtacaaaaaagcaggcttaaacaatggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgagcaagaagatcggcctgttttacggcacgcaaaccggcaaaaccgagagcgtcgcggagatcattagggacgagttcgggaacgacgtggtgacactccacgacgtcagccaagcggaggttacggacctgaacgactaccagtacctgatcatcgggtgccccacctggaatataggcgagttgcaatcggactgggagggcttgtactctgagcttgacgacgtggacttcaacgggaagctggtggcttacttcggtacgggtgaccagattggctacgcagacaacttccaggacgccataggcatcctcgaggagaagatcagccagagaggtgggaaaaccgtgggttactggagcactgacggctacgacttcaacgactcgaaggcgttgaggaacggcaagttcgtgggtcttgcgctcgacgaggacaaccagtccgatctcacagacgacaggatcaagtcctgggtggctcagctgaaatccgagttcggcctttag

SEQ ID NO: 21 - DNA, 1907 bp, Artificial sequence
  the promoter of SEQ ID NO 7 combined with the nucleic acid of SEQ ID NO 3 for the transit peptide of SEQ ID NO 4 and the nucleic acid of SEQ ID NO 15 coding for the flavodoxin of SEQ ID NO 16, with a short linker sequence between the promoter & CDS
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacaccaatttaaatcaactagggatatcacaagtttgtacaaaaaagcaggcttaaacaatggctgctgcagtaacagccgcagtctccttgccatactccaactccacttcccttccgatcagaacatctattgttgcaccagagagacttgtcttcaaaaaggtttcattgaacaatgtttctataagtggaagggtaggcaccatcagagctctcataatgacaaaaattggacttttttacggtactcaaaccggcaacactgaaaccattgctgaactgattcaaaaagaaatgggcggcgatagtgtggtcgatatgatggatatatcccaggctgatgttgatgattttaggcaatatagttgcctgattatcggttgtcccacctggaatgtgggggaactccagagtgattgggaaggcttttatgaccaattagacgaaattgattttaatggcaaaaaagtagcctattttggtgctggcgatcaggttggttatgcagataattttcaagacgccatgggcattttagaagaaaaaatcagtggattaggcggtaaaacagtggggttttggcccaccgctggctatgattttgacgaatcaaaagcggtgaaaaatgggaaatttgttggtttagctttggacgaagataatcagccagagttaacagaattaagagtaaagacatgggtaagtgaaattaaaccaattttgcaatcctaa

(19) United States
(12) Patent Application Publication
(10) Pub. No.: US 2015/0059735 A1
(43) Pub. Date: Mar. 5, 2015

(54) PLANTS HAVING ONE OR MORE ENHANCED YIELD-RELATED TRAITS AND METHOD FOR MAKING SAME

(71) Applicant: BASF Plant Science Company GmbH, Ludwigshafen (DE)

(72) Inventor: Christophe Reuzeau, La Chapelle Gonaguet (FR)

(73) Assignee: BASF Plant Science Company, Ludwigshafen (DE)

(21) Appl. No.: 14/389,917

(22) PCT Filed: Mar. 15, 2013
(86) PCT No.: PCT/IB2013/052071

§ 371 Date: Oct. 1, 2014

(30) Foreign Application Priority Data
Apr. 2, 2012 (EP) 12162830.9

(51) Int. Cl. C12N 15/82; C13B 10/00

(52) U.S. Cl. CPC C12N 15/8201; C13B 10/00
USPC 127/43; 800/284; 800/290

(57) ABSTRACT

A method for enhancing various economically important yield-related traits in plants by modulating expression of a nucleic acid encoding a flavodoxin polypeptide in plants in a specific way. Provided are plants having the expression of a nucleic acid encoding a flavodoxin polypeptide modulated by a particular type of promoter, which plants have enhanced yield-related traits compared with control plants. Hitherto unknown constructs, which are useful in performing the methods of the invention, are also provided.

The present application claims priority of the following applications: EP 12162830.9 filed on Apr. 2, 2012 and U.S. 61/618,859 filed on Apr. 2, 2012 all of which are herewith incorporated by reference with respect to the entire disclosure content. Incorporated by reference are further, international patent application PCT/GB02/04612, published as WO2003/035881, explicitly the pages 35 to 45 and particularly the flavodoxins and transit peptides listed therein; as well as EP1532257 and particularly the PCPR promoter disclosed therein as “PRO123” and as SEQ ID NO: 14.

### BACKGROUND OF THE INVENTION

1. Field of the Invention

The present invention relates generally to the field of plant molecular biology and concerns a method for enhancing one or more yield-related traits in plants by increasing expression in a plant of a nucleic acid encoding a flavodoxin polypeptide in a particular way. The present invention also concerns plants having specifically increased expression of an exogenous nucleic acid encoding a flavodoxin polypeptide with plastid targeting, which plants have one or more enhanced yield-related traits relative to corresponding control plants. The invention also provides constructs useful in the methods, uses, plants, harvestable parts and products of the invention of the invention.

2. Description of Related Art

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

One trait of economic interest is increased yield. Yield is normally defined as the measurable produce of economic value from a crop. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the abovementioned factors may therefore contribute to increasing crop yield.

Seed yield is an important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, nutrient deficiency, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Environmental stress is a major limiting factor for plant productivity and crop yield. Many of the deleterious processes undergone by plants exposed to adverse environmental conditions are mediated by reactive oxygen species (ROS) which are generated in Chloroplasts through the faulty performance of the photosynthetic apparatus (Foyer, C. H. et al. (1994) Plant Cell Environ. 17, 507-523, Hammond-Kosack, K. E., and Jones, J. D. G. (1996) Plant Cell 8, 1773-1791, Allen, R. (1995) Plant Physiol. 107/1049-1054), auto-oxidation of components of the photosynthetic electron transport chain leads to the formation of superoxide radicals and their derivatives, hydrogen peroxide and hydroxyl radicals. These compounds react with a wide variety of biomolecules (most conspicuously, DNA), causing cell stasis and death.

To cope with the damaging effects of reactive oxygen species (ROS), aerobic organisms have evolved highly efficient antioxidant defense systems which are made up of both enzymatic and non-enzymatic constituents. In different tissues and organisms, antioxidants play different and often complementary protective functions, such as direct scavenging of ROS 1 replacement of damaged oxidant sensitive biomolecules and DNA repair activities (Fridovich 11. (1997). J. Biol. Chem. 272, 1851-1857). At least part of the cellular response against oxidative stress is of an adaptive nature and involves de novo synthesis of committed members of the antioxidant barrier. Various multigenic responses have been recognized in the facultative aerobic bacterium Escherichia coli, including those modulated by the soxRS and oxyR regulons (Hidalgo, E., and Demple, B. (1996). In Regulation of Gene Expression in Escherichia coli, Molecular Biology Intelligence Unit Series (E. C. C. Lin and A. S. Lynch, eds.), pp. 434-452, Austin, Tex.: R. G. Landis).

The soxRS response appears to be specifically tailored to face the challenges imposed by exposure of the cells to superoxide radicals or to nitric oxide. Many different components of the response have been identified, including two soluble flavoproteins: FAD-containing ferredoxin-NADP+ reductase (FNR), and its electron partner substrate flavodoxin (Liochev et al. (1994) Proc. Natl. Acad. Sei. USA 91, 1328-1331, Zheng, M. et al (1999) J. Bacteriol. 181, 4639-4643).

Flavodoxins are small monomeric proteins (Mw 18,800) containing one molecule of non-covalently bound FMN (Razquin, P. et al (1988) J. Bacteriol. 176, 7409-7411). FNR is able to use, with roughly similar efficiencies, both flavodoxin and the iron-sulfur protein ferredoxin as substrates for its NADP(H) oxidoreductase activity. In cyanobacteria, flavodoxin expression is induced under conditions of iron deprivation, when ferredoxin cannot be synthesized.

As part of the soxRS response of E. coli, both FNR and flavodoxin levels increase over twenty times upon treatment of the bacteria with superoxide-propagating compounds such as the redox cycling herbicide methyl vialogen (MV), whereas ferredoxin amounts are not affected (Rodriguez, R. E. et al (1998) Microbiology 144, 2375-2376). Unlike FNR and ferredoxins, which are widely distributed among plastids, mitochondria and bacteria, flavodoxin occurrence appears to be largely restricted to bacteria. Flavodoxins have not been isolated from plant tissues, and no flavodoxin homologue has been recognized in the Arabidopsis thaliana genome (The Arabidopsis Genome Initiative (2000) Nature 408, 796-815). It has been described that plant lines which have been engineered to express a flavoprotein such as flavodoxin display enhanced tolerance compared to control, untreated plants, when exposed to an environmental stress condition (see the international patent application PCT/GB02/04612, published as WO2003/035881, filed on Oct. 10, 2002 by the applicant PLANT BIOSCIENCE LTD, in the following referred to as WO 03/035881).

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

It has now been found that various yield-related traits may be improved in plants by modulating expression in a plant of a nucleic acid encoding a flavodoxin polypeptide.

### BRIEF SUMMARY OF THE INVENTION

The present invention concerns a method for enhancing one or more yield-related traits in plants by specifically increasing the expression in a plant of a nucleic acid encoding a flavodoxin polypeptide that is targeted to plastids. The present invention also concerns plants having specifically increased expression of a nucleic acid encoding a flavodoxin polypeptide with plastid targeting, which plants have one or more enhanced yield-related traits compared with control plants. The invention also provides hitherto unknown constructs comprising flavodoxin-encoding nucleic acids, useful in performing the methods of the invention.

A preferred embodiment is a method for enhancing one or more yield-related traits in a plant relative to control plants, comprising the steps of increasing the expression, preferably by recombinant methods, in a plant of an exogenous nucleic acid encoding a transit peptide and a flavodoxin polypeptide in a particular way, wherein the expression is under the control of a particular promoter sequence operably linked to the nucleic acid encoding the transit peptide and the flavodoxin polypeptide, and growing the plant(s).

Hence, it is an object of the invention to provide an expression construct and a vector construct comprising a nucleic acid encoding a transit peptide and a flavodoxin polypeptide, operably linked to a beneficial promoter sequence. The use of such genetic constructs for making a transgenic plant having one or more enhanced yield-related traits, preferably increased biomass and/or seed yield, relative to control plants is provided.

Also a preferred embodiment are transgenic plants transformed with one or more expression constructs of the invention, and thus, expressing in a particular way the nucleic acids encoding a transit peptide and a flavodoxin protein, wherein the plants have one or more enhanced yield-related trait. Harvestable parts of the transgenic plants of the present invention and products derived from the transgenic plants and their harvestable parts are also part of the present invention.

In particular it has been found that surprisingly the expression of an exogenous nucleic acid encoding for a transit peptide and a flavodoxin as defined herein under the control of a Protochlorophyllide Reductase Promoter (PCPR) as defined herein below results in increased biomass, increased seed yield and/or increased sugar content of plants comprising said combination of PCPR promoter functionally linked to said exogenous nucleic acid compared with control plants under standard and/or abiotic stress conditions.

### BRIEF DESCRIPTION OF THE SEVERAL VIEWS OF THE DRAWINGS

The present invention will now be described with reference to the following figures in which:

FIG. 1 represents the domain structure of SEQ ID NO: 2 with conserved motifs and or domains. The domains were identified and visualized using the software InterProScan (see Zdobnov E. M. and Apweiler R.; “InterProScan—an integration platform for the signature-recognition methods in InterPro.”; Bioinformatics, 2001, 17(9): 847-8; InterPro database, release Release 36.0, 23 Feb. 2012) (A) and the InterproScan software version 4.8, InterPro database release 41 of Feb. 13, 2013 (B).

FIG. 2 represents the binary vector used for specific expression in sugarcane of a nucleic acid encoding flavodoxin (FLD) fused to a plastid transit peptide (TP), represented by TP::FLD, under the control of a protochlorophyllide reductase promoter promoter (pPCPR). Flavodoxin, transit peptide and protochlorophyllide reductase promoter promoter are as disclosed herein.

### DETAILED DESCRIPTION OF THE INVENTION

### Definitions

The following definitions will be used throughout the present application. The section captions and headings in this application are for convenience and reference purpose only and should not affect in any way the meaning or interpretation of this application. The technical terms and expressions used within the scope of this application are generally to be given the meaning commonly applied to them in the pertinent art of plant biology, molecular biology, bioinformatics and plant breeding. All of the following term definitions apply to the complete content of this application. It is to be understood that as used in the specification and in the claims, “a” or “an” can mean one or more, depending upon the context in which it is used. Thus, for example, reference to “a cell” can mean that at least one cell can be utilized.

The term “essentially”, “about”, “approximately” and the like in connection with an attribute or a value, particularly also define exactly the attribute or exactly the value, respectively. The term “about” in the context of a given numeric value or range relates in particular to a value or range that is within 20%, within 10%, or within 5% of the value or range given. As used herein, the term “comprising” also encompasses the term “consisting of”.

### Peptide(s)/Protein(s)

The terms “peptides”, “oligopeptides”, “polypeptide” and “protein” are used interchangeably herein and refer to amino acids in a polymeric form of any length, linked together by peptide bonds, unless mentioned herein otherwise.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Homologue(s)

“Homologues” of a protein encompass peptides, oligopeptides, polypeptides, proteins and enzymes having amino acid substitutions, deletions and/or insertions relative to the unmodified protein in question and having substantially the same biological and functional activity as the unmodified protein from which they are derived. “Homologues” of a gene encompass genes having a nucleic acid sequence with nucleotide substitutions, deletions and/or insertions relative to the unmodified gene in question and having substantially the same biological and/or functional activity as the unmodified gene from which they are derived, or encoding polypeptides having substantially the same biological and functional activity as the polypeptide encoded by the unmodified nucleic acid sequence.

The term “nucleotide” refers to a nucleic acid building block consisting of a nucleobase, a pentose and at least one phosphate group. Thus, the term “nucleotide” includes a nukleosidmonophosphate, nukleosiddiphosphate, and nukleosidtriphosphate.

Orthologues and paralogues are two different forms of homologues and encompass evolutionary concepts used to describe the ancestral relationships of genes or proteins. Paralogues are genes or proteins within the same species that have originated through duplication of an ancestral gene or protein; orthologues are genes or protein from different organisms that have originated through speciation, and are also derived from a common ancestral gene or protein.

A “deletion” refers to removal of one or more amino acids from a protein or a removal of one or more nucleotides from a nucleic acid.

An “insertion” refers to one or more amino acid residues being introduced into a predetermined site in a protein or to one or more nucleotides being introduced into a predetermined site in a nucleic acid sequence. Regarding a protein, insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope.

A “substitution” refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols (see Current Protocols in Molecular Biology, John Wiley & Sons, N.Y. (1989 and yearly updates)).

### Derivatives

“Derivatives” of proteins or polypeptides include polypeptides which may, compared to the amino acid sequence of the naturally-occurring form of the protein or polypeptide, such as the protein of interest, comprise substitutions of amino acids with non-naturally occurring amino acid residues, or additions of non-naturally occurring amino acid residues. “Derivatives” of a protein or polypeptide also encompass polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003). “Derivatives” of nucleic acids include nucleic acids which may, compared to the nucleotide sequence of the naturally-occurring form of the nucleic acid comprise deletions, alterations, or additions with non-naturally occurring nucleotides. “Derivatives” of a nucleic acid also encompass nucleic acids which comprise naturally occurring altered or non-naturally altered nucleotides as compared to the nucleotide sequence of a naturally-occurring form of the nucleic acid. A derivative of a protein or nucleic acid still provides substantially the same function, e.g., enhanced yield-related trait, when expressed or repressed in a plant respectively.

### Functional Fragments

The term “functional fragment” refers to any nucleic acid or protein which comprises merely a part of the full-length nucleic acid or full-length protein, respectively, but still provides substantially the same function, e.g., enhanced yield-related trait, when expressed or repressed in a plant respectively.

In cases where overexpression of nucleic acid is desired, the term “substantially the same functional activity” or “substantially the same function” means that any homologue and/or fragment provide increased/enhanced yield-related trait(s) when expressed in a plant. Preferably substantially the same functional activity or substantially the same function means at least 50%, at least 60%, at least 70%, at least 80%, at least 90%, at least 95%, at least 98%, at least 99% or 100% or higher increased/enhanced yield-related trait(s) compared with functional activity provided by the exogenous expression of the full-length flavodoxin nucleotide sequence or the flavodoxin amino acid sequence.

### Domain/Motif/Consensus Sequence/Signature

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” or “signature” refers to a short conserved region in the sequence of evolutionarily related amino acid or nucleic acid sequences. For amino acid sequences motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)) and the Pfam protein families database (R. D. Finn, J. Mistry, J. Tate, P. Coggill, A. Heger, J. E. Pollington, O. L. Gavin, P. Gunesekaran, G. Ceric, K. Forslund, L. Holm, E. L. Sonnhammer, S. R. Eddy, A. Bateman Nucleic Acids Research (2010) Database Issue 38:211-222). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percentage sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol. 147(1); 195-7).

### Reciprocal BLAST

Typically, this involves a first BLAST involving BLASTing (i.e. running the BLAST software with the sequence of interest as query sequence) a query sequence (for example using any of the sequences listed in Table 2 or 3) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived. The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

### Transit Peptide

A “transit peptide” (or transit signal, signal peptide, signal sequence) is a short (3-60 amino acids long) peptide chain that directs the transport of a protein, preferably to organelles within the cell or to certain subcellular locations or for the secretion of a protein. Transit peptides may also be called transit signal, signal peptide, signal sequence, targeting signals, or (subcellular) localization signals.

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tₘ may be calculated using the following equations, depending on the types of hybrids:

1) DNA-DNA hybrids (Meinkoth and Wahl, Anal. Biochem., 138: 267-284, 1984): Tₘ=81.5° C.+16.6×log₁₀[Na⁺]^{a}+0.41×%[G/C^{b}]−500×[L^{c}]⁻¹−0.61×% formamide
2) DNA-RNA or RNA-RNA hybrids:
Tₘ=79.8° C.+18.5 (log₁₀[Na⁺]^{a})+0.58 (% G/C^{b})+11.8 (% G/C^{b})²−820/L^{c}
3) oligo-DNA or oligo-RNA^{d} hybrids:
For <20 nucleotides: Tₘ=2 (Iₙ)
For 20-35 nucleotides: Tₘ=22+1.46 (Iₙ) ^{a} or for other monovalent cation, but only accurate in the 0.01-0.4 M range.^{b} only accurate for % GC in the 30% to 75% range.^{c} L=length of duplex in base pairs.^{d} oligo, oligonucleotide; Iₙ,=effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. In a preferred embodiment high stringency conditions mean hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC. For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

“Alleles” or “allelic variants” are alternative forms of a given gene, located at substantially the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Endogenous

Reference herein to an “endogenous” nucleic acid and/or protein refers to the nucleic acid and/or protein in question as found in a plant in its natural form (i.e., without there being any human intervention like recombinant DNA engineeringtechnology), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Exogenous

The term “exogenous” (in contrast to “endogenous”) nucleic acid or gene refers to a nucleic acid that has been introduced in a plant by means of recombinant DNA technology. An “exogenous” nucleic acid can either not occur in a plant in its natural form, be different from the nucleic acid in question as found in a plant in its natural form, or can be identical to a nucleic acid found in a plant in its natural form, but integrated not within its natural genetic environment. The corresponding meaning of “exogenous” is applied in the context of protein expression. For example, a transgenic plant containing a transgene, i.e., an exogenous nucleic acid, may, when compared to the expression of the endogenous gene, encounter a substantial increase of the expression of the respective gene or protein in total. A transgenic plant according to the present invention includes an exogenous flavodoxin nucleic acid integrated at any genetic loci and optionally the plant may also include the endogenous gene within the natural genetic background.

Gene shuffling/Directed Evolution

“Gene shuffling” or “directed evolution” consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Expression Cassette

“Expression cassette” as used herein is DNA capable of being expressed in a host cell or in an in-vitro expression system. Preferably the DNA, part of the DNA or the arrangement of the genetic elements forming the expression cassette is artificial. The skilled artisan is well aware of the genetic elements that must be present in the expression cassette in order to be successfully expressed. The expression cassette comprises a sequence of interest to be expressed operably linked to one or more control sequences (at least to a promoter) as described herein. Additional regulatory elements may include transcriptional as well as translational enhancers, one or more NEENA as described herein, and/or one or more RENA as described herein. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section for increased expression/overexpression. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The expression cassette may be integrated into the genome of a host cell and replicated together with the genome of said host cell.

### Construct/Genetic Construct

Artificial This is DNA (such as but, not limited to plasmids or viral DNA)—artificial in part or total or artificial in the arrangement of the genetic elements contained—capable of increasing or decreasing the expression of DNA and/or protein of interest typically by replication in a host cell and used for introduction of a DNA sequence of interest into a host cell or host organism. Replication may occur after integration into the host cell's genome or through the presence of the construct as part of a vector or an artificial chromosome inside the host cell. Host cells of the invention may be any cell selected from bacterial cells, such as Escherichia coli or Agrobacterium species cells, yeast cells, fungal, algal or cyanobacterial cells or plant cells. The skilled artisan is well aware of the genetic elements that must be present on the genetic construct in order to successfully transform, select and propagate host cells containing the sequence of interest.

Typically the construct/genetic construct is an expression construct and comprises one or more expression cassettes that may lead to overexpression (overexpression construct) or reduced expression of a gene of interest. A construct may consist of an expression cassette. The sequence(s) of interest is/are operably linked to one or more control sequences (at least to a promoter) as described herein. Additional regulatory elements may include transcriptional as well as translational enhancers, one or more NEENA as described herein, and/or one or more RENA as described herein. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section for increased expression/overexpression. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

### Vector Construct/Vector

This is DNA (such as but, not limited to plasmids or viral DNA)—artificial in part or total or artificial in the arrangement of the genetic elements contained—capable of replication in a host cell and used for introduction of a DNA sequence of interest into a host cell or host organism. A vector may be a construct or may comprise at least one construct. A vector may replicate without integrating into the genome of a host cell, e.g. a plasmid vector in a bacterial host cell, or it may integrate part or all of its DNA into the genome of the host cell and thus lead to replication and expression of its DNA. Host cells of the invention may be any cell selected from bacterial cells, such as Escherichia coli or Agrobacterium species cells, yeast cells, fungal, algal or cyanobacterial cells or plant cells. The skilled artisan is well aware of the genetic elements that must be present on the genetic construct in order to successfully transform, select and propagate host cells containing the sequence of interest. Typically the vector comprises at least one expression cassette. The one or more sequence(s) of interest is operably linked to one or more control sequences (at least to a promoter) as described herein. Additional regulatory elements may include transcriptional as well as translational enhancers, one or more NEENA as described herein and/or one or more RENA as described herein. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

### Regulatory Element/Control Sequence/Promoter/Promoter Sequence

The terms “regulatory element”, “control sequence”, “promoter”, and “promoter sequence” refer to regulatory nucleic acid sequences capable of effecting expression of the associated sequences. Regulatory elements may be promoter(s). The terms “promoter” and “promoter sequences” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described herein, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” or “functionally linked” is used interchangeably and, as used herein, refers to a functional linkage between the promoter sequence and the gene of interest, such that the promoter sequence is able to direct transcription of the gene of interest.

The term “functional linkage” or “functionally linked” with respect to regulatory elements, is to be understood as meaning, for example, the sequential arrangement of a regulatory element (e.g. a promoter) with a nucleic acid sequence to be expressed and, if appropriate, further regulatory elements (such as e.g., a terminator, NEENA as described herein or a RENA as described herein) in such a way that each of the regulatory elements can fulfil its intended function to allow, modify, facilitate or otherwise influence expression of said nucleic acid sequence. As a synonym the wording “operable linkage” or “operably linked” may be used. The expression may result, depending on the arrangement of the nucleic acid sequences, in sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed is recombinantly positioned behind the sequence acting as promoter, so that the two sequences are linked covalently to each other. The distance between the promoter sequence and the recombinant nucleic acid sequence to be expressed is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch EF and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Constitutive promoterA “constitutive promoter” refers to a promoter that is transcriptionally active during most, but not necessarily all, phases of growth and development and under most environmental conditions, in at least one cell, tissue or organ.

A “ubiquitous promoter” is active in substantially all tissues or cells of an organism.

A “developmentally-regulated promoter” is active during certain developmental stages or in parts of the plant that undergo developmental changes.

An “inducible promoter” has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

An “organ-specific” or “tissue-specific promoter” is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

A “seed-specific promoter” is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific.

A “green tissue-specific promoter” as defined herein is a promoter that is transcriptionally active predominantly in green tissue, substantially to the exclusion of any other parts of a plant, whilst still allowing for any leaky expression in these other plant parts.

Another example of a tissue-specific promoter is a meristem-specific promoter, which is transcriptionally active predominantly in meristematic tissue, substantially to the exclusion of any other parts of a plant, whilst still allowing for any leaky expression in these other plant parts.

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta® aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, genetice construct or a vector comprising the nucleic acid sequence or an organism transformed with the nucleic acid sequences, expression cassettes or vectors according to the invention, all those constructions brought about by recombinant methods in which either
(a) the sequences of the flavodoxin nucleic acids or a part thereof, or
(b) genetic control sequence(s) which is operably linked with the flavodoxin nucleic acid sequence according to the invention, for example a promoter, or
(c) a) and b)
   are not located in their natural genetic environment or have been modified by recombinant methods e.g. modified and/or inserted by man for example by genetechnological methods. The modification may take the form of, for example, a substitution, addition, deletion, inversion or insertion of one or more nucleotide residues. The natural genetic environment is understood as meaning the natural genomic or chromosomal locus in the original plant or the presence in a genomic library or the combination with the natural promoter.

Also linking a nucleic acid sequence encoding a transit peptide for plastid targeting with a nucleic acid encoding flavodoxin as defined herein that is naturally not linked to said transit peptide creates a recombinant sequence.

A recombinant nucleic acid, expression cassette, genetic construct or vector construct preferably comprises a natural gene and a natural promoter, a natural gene and a non-natural promoter, a non-natural gene and a natural promoter, or a non-natural gene and a non-natural promoter.

In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp.

A naturally occurring expression cassette—for example the naturally occurring combination of the natural promoter of the nucleic acid sequences with the corresponding nucleic acid sequence encoding a protein useful in the methods of the present invention, as defined above—becomes a recombinant expression cassette when this expression cassette is modified by man by non-natural, synthetic (“artificial”) methods such as, for example, mutagenic treatment. Suitable methods are described, for example, in U.S. Pat. No. 5,565,350, WO 00/15815 or US200405323. Furthermore, a naturally occurring expression cassette—for example the naturally occurring combination of the natural promoter of the nucleic acid sequences with the corresponding nucleic acid sequence encoding a protein useful in the methods of the present invention, as defined above—becomes a recombinant expression cassette when this expression cassette is not integrated in the natural genetic environment but in a different genetic environment.

It shall further be noted that in the context of the present invention, the term “isolated nucleic acid” or “isolated protein” may in some instances be considered as a synonym for a “recombinant nucleic acid” or a “recombinant protein”, respectively, and refers to a nucleic acid or protein that is not located in its natural genetic environment and cellular environment, respectively. The isolated gene may be isolated from an organism or may be manmade, for example by chemical synthesis. In one embodiment an isolated nucleic acid sequence or isolated nucleic acid molecule is one that is not in its native surrounding or its native nucleic acid neighbourhood, yet is physically and functionally connected to other nucleic acid sequences or nucleic acid molecules and is found as part of a nucleic acid construct, vector sequence or chromosome.

As used herein, the term “transgenic” relating to an organisms e.g. transgenic plant refers to an organism, e.g., a plant, plant cell, callus, plant tissue, or plant part that exogenously contains the nucleic acid, construct, vector or expression cassette described herein or a part thereof which is preferably introduced by processes that are not essentially biological, preferably by Agrobacteria-mediated transformation or particle bombardment. A transgenic plant for the purposes of the invention is thus understood as meaning, as above, that the nucleic acids described herein are not present in, or not originating from the genome of said plant, or are present in the genome of said plant but not at their natural genetic environment in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning that the expression of naturally in that plant occurring nucleic acid sequences at an unnatural genetic environment in the genome, i.e. homologous expression, or that heterologous expression of not naturally in that plant occurring nucleic acid sequences takes place. Preferred transgenic plants are mentioned herein.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. For the purposes of this invention, the original unmodulated expression may also be absence of any expression. The term “modulating the activity” or the term “modulating expression” shall mean any change of the expression of the inventive nucleic acid sequences and/or encoded proteins, which leads to increased or decreased yield-related trait(s) such as but not limited to increased or decreased seed yield and/or increased or decreased growth of the plants. The expression can increase from zero (absence of, or immeasurable expression) to a certain amount, or can decrease from a certain amount to immeasurable small amounts or zero.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product. The term “expression” or “gene expression” can also include the translation of the mRNA and therewith the synthesis of the encoded protein, i.e., protein expression.

### Increased Expression/Enhanced Expression/Overexpression

The term “increased expression”, “enhanced expression”, or “overexpression” as used herein means any form of expression that is additional to the original wild-type expression level. For the purposes of this invention, the original wild-type expression level might also be zero, i.e. absence of expression or immeasurable expression. Reference herein to “increased expression”, “enhanced expression” or “overexpression” is taken to mean an increase in gene expression and/or, as far as referring to polypeptides, increased polypeptide levels and/or increased polypeptide activity, relative to control plants. The increase in expression, polypeptide levels or polypeptide activity is in increasing order of preference at least 10%, 20%, 30%, 40% or 50%, 60%, 70%, 80%, 85%, 90%, or 100% or even more compared to that of control plants. The increase in expression may be in increasing order of preference at least 100%, 200%, 300%, 400%, 500%, 600%, 700%, 800%, 900%, 1000%, 2000%, 3000%, 4000% or 5000% or even more compared to that of control plants. In cases when the control plants have only very little expression, polypeptide levels or polypeptide activity of the sequence in question and/or the recombinant gene is under the control of strong regulatory element(s) the increase in expression, polypeptide levels or polypeptide activity may be at least 100 times, 200 times, 300 times, 400 times, 500 times, 600 times, 700 times, 800 times, 900 times, 1000 times, 2000 times, 3000 times, 5000 times, 10 000 times, 20 000 times, 50 000 times, 100 000 times or even more compared to that of control plants.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell. biol. 8: 4395-4405; Callis et al. (1987) Genes Dev 1:1183-1200). Such intron enhancement of gene expression is typically greatest when placed near the 5′ end of the transcription unit. Use of the maize introns Adh1-5 intron 1, 2, and 6, the Bronze-1 intron are known in the art. For general information see: The Maize Handbook, Chapter 116, Freeling and Walbot, Eds., Springer, N.Y. (1994).

To obtain increased expression or overexpression of a polypeptide most commonly the nucleic acid encoding this polypeptide is overexpressed in sense orientation with a polyadenylation signal. Introns or other enhancing elements may be used in addition to a promoter suitable for driving expression with the intended expression pattern. In contrast to this, overexpression of the same nucleic acid sequence as antisense construct will not result in increased expression of the protein, but decreased expression of the protein.

### Decreased Expression

Reference herein to “decreased expression” or “reduction or substantial elimination of expression” is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or even more compared to that of control plants.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. Alternatively, a plant cell that cannot be regenerated into a plant may be chosen as host cell, i.e. the resulting transformed plant cell does not have the capacity to regenerate into a (whole) plant.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:1-9; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229). The genetically modified plant cells can be regenerated via all methods with which the skilled worker is familiar. Suitable methods can be found in the abovementioned publications by S. D. Kung and R. Wu, Potrykus or Hofgen and Willmitzer. Alternatively, the genetically modified plant cells are non-regenerable into a whole plant.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described herein.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Throughout this application a plant, plant part, seed or plant cell transformed with—or interchangeably transformed by—a construct or transformed with or by a nucleic acid is to be understood as meaning a plant, plant part, seed or plant cell that carries said construct or said nucleic acid as a transgene due the result of an introduction of said construct or said nucleic acid by biotechnological means. The plant, plant part, seed or plant cell therefore comprises said recombinant construct or said recombinant nucleic acid. Any plant, plant part, seed or plant cell that no longer contains said recombinant construct or said recombinant nucleic acid after introduction in the past, is termed null-segregant, nullizygote or null control, but is not considered a plant, plant part, seed or plant cell transformed with said construct or with said nucleic acid within the meaning of this application.

### T-DNA Activation Tagging

“T-DNA activation” tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### TILLING

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

### Homologous Recombination

“Homologous recombination” allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield-Related Trait(s)

A “yield-related trait” is a trait or feature which is related to plant yield. Yield-related traits may comprise one or more of the following non-limitative list of features: early flowering time, yield, biomass, seed yield, early vigour, greenness index, growth rate, agronomic traits, such as e.g. tolerance to submergence (which leads to increased yield in rice), Water Use Efficiency (WUE), Nitrogen Use Efficiency (NUE), etc. The term “one or more yield-related traits” is to be understood to refer to one yield-related trait, or two, or three, or four, or five, or six or seven or eight or nine or ten, or more than ten yield-related traits of one plant compared with a control plant.

Reference herein to “enhanced yield-related trait” is taken to mean an increase relative to control plants in a yield-related trait, for instance in early vigour, seed yield and/or in biomass, of a whole plant or of one or more parts of a plant, which may include (i) above-ground parts, preferably aboveground harvestable parts, and/or (ii) parts below ground, preferably harvestable parts below ground.

In particular, such harvestable parts are roots such as taproots, stems, beets, tubers, leaves, flowers or seeds, and performance of the methods of the invention results in plants having increased seed yield relative to the seed yield of control plants, and/or increased aboveground biomass, in particular stem biomass relative to the aboveground biomass, and in particular stem biomass of control plants, and/or increased root biomass relative to the root biomass of control plants and/or increased beet biomass relative to the beet biomass of control plants. Moreover, it is particularly contemplated that the sugar content (in particular the sucrose content) in the above ground parts, particularly stem (in particular of sugarcane plants) and/or in the belowground parts, in particular in roots including taproots, and tubers, and/or in beets (in particular in sugar beets) is increased relative to the sugar content (in particular the sucrose content) in corresponding part(s) of the control plant.

Throughout the present application the tolerance of and/or the resistance to one or more agrochemicals by a plant, e.g. herbicide tolerance, is not considered a yield-related trait within the meaning of this term of the present application. An altered tolerance of and/or the resistance to one or more agrochemicals by a plant, e.g. improved herbicide tolerance, is not an “enhanced yield-related trait” as used throughout this application.

In a particular embodiment of the present invention, any reference to one or more enhanced yield-related trait(s) is meant to exclude the restoration of the expression and/or activity of the POI polypeptide in a plant in which the expression and/or the activity of the POI polypeptide has been reduced or disabled when compared to the original wildtype plant or original variety. For example, the overexpression of the POI polypeptide in a knock-out mutant variety of a plant, wherein said POI polypeptide or an orthologue or paralogue has been knocked-out is not considered enhancing one or more yield-related trait(s) within the meaning of the current invention.

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters.

The terms “yield of a plant” and “plant yield” are used interchangeably herein and are meant to refer to vegetative biomass such as root and/or shoot biomass, to reproductive organs, and/or to propagules such as seeds of that plant.

Flowers in maize are unisexual; male inflorescences (tassels) originate from the apical stem and female inflorescences (ears) arise from axillary bud apices. The female inflorescence produces pairs of spikelets on the surface of a central axis (cob). Each of the female spikelets encloses two fertile florets, one of them will usually mature into a maize kernel once fertilized. Hence a yield increase in maize may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate, which is the number of filled florets (i.e. florets containing seed) divided by the total number of florets and multiplied by 100), among others.

Inflorescences in rice plants are named panicles. The panicle bears spikelets, which are the basic units of the panicles, and which consist of a pedicel and a floret. The floret is borne on the pedicel and includes a flower that is covered by two protective glumes: a larger glume (the lemma) and a shorter glume (the palea). Hence, taking rice as an example, a yield increase may manifest itself as an increase in one or more of the following: number of plants per square meter, number of panicles per plant, panicle length, number of spikelets per panicle, number of flowers (or florets) per panicle; an increase in the seed filling rate which is the number of filled florets (i.e. florets containing seeds) divided by the total number of florets and multiplied by 100; an increase in thousand kernel weight, among others.

### Early Flowering Time

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can for instance be determined using the method as described in WO 2007/093444.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increased Growth Rate

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a mature seed up to the stage where the plant has produced mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

### Seed Yield

Increased seed yield may manifest itself as one or more of the following:
a) an increase in seed biomass (total seed weight) which may be on an individual seed basis and/or per plant and/or per square meter;
b) increased number of flowers per plant;
c) Increased number of seeds;
d) increased seed filling rate (which is expressed as the ratio between the number of filled florets divided by the total number of florets);
e) increased harvest index, which is expressed as a ratio of the yield of harvestable parts, such as seeds, divided by the biomass of aboveground plant parts; and
f) increased thousand kernel weight (TKW), which is extrapolated from the number of seeds counted and their total weight. An increased TKW may result from an increased seed size and/or seed weight, and may also result from an increase in embryo and/or endosperm size.

The terms “filled florets” and “filled seeds” may be considered synonyms.

An increase in seed yield may also be manifested as an increase in seed size and/or seed volume. Furthermore, an increase in seed yield may also manifest itself as an increase in seed area and/or seed length and/or seed width and/or seed perimeter.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Biomass

The term “biomass” as used herein is intended to refer to the total weight of a plant or plant part. Total weight can be measured as dry weight, fresh weight or wet weight. Within the definition of biomass, a distinction may be made between the biomass of one or more parts of a plant, which may include any one or more of the following:
aboveground parts such as but not limited to shoot biomass, seed biomass, leaf biomass, etc.;
aboveground harvestable parts such as but not limited to shoot biomass, seed biomass, stem biomass, leaf biomass, setts etc.;
parts below ground, such as but not limited to root biomass, tubers, bulbs, etc.;
harvestable parts below ground, such as but not limited to root biomass, tubers, bulbs, etc.;
harvestable parts partially below ground such as but not limited to beets and other hypocotyl areas of a plant, rhizomes, stolons or creeping rootstalks;
vegetative biomass such as root biomass, shoot biomass, etc.;
reproductive organs; and
propagules such as seed.

### Root

In a preferred embodiment throughout this application any reference to “root” as biomass or harvestable parts or as organ, e.g., of increased sugar content, is to be understood as a reference to harvestable parts partly inserted in or in physical contact with the ground such as but not limited to beets and other hypocotyl areas of a plant, rhizomes, stolons or creeping rootstalks, as well as harvestable parts belowground, such as but not limited to root, taproot, tubers or bulbs, but not including leaves.

### Stress Resistance

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35%, 30% or 25%, more preferably less than 20% or 15% in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture.

“Biotic stress” is understood as the negative impact done to plants by other living organisms, such as bacteria, viruses, fungi, nematodes, insects, other animals or other plants. “Biotic stresses” are typically those stresses caused by pathogens, such as bacteria, viruses, fungi, plants, nematodes and insects, or other animals, which may result in negative effects on plant growth and/or yield.

“Abiotic stress” is understood as the negative impact of non-living factors on the living plant in a specific environment. “Abiotic stresses” may be due to drought or excess water, anaerobic stress, salt stress, chemical toxicity, oxidative stress and hot, cold or freezing temperatures. The “abiotic stress” may be an osmotic stress caused by a water stress, e.g. due to drought, salt stress, or freezing stress. Abiotic stress may also be an oxidative stress or a cold stress. “Freezing stress” is intended to refer to stress due to freezing temperatures, i.e. temperatures at which available water molecules freeze and turn into ice. “Cold stress”, also called “chilling stress”, is intended to refer to cold temperatures, e.g. temperatures below 10°, or preferably below 5° C., but at which water molecules do not freeze. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

### Increase/Improve/Enhance

The terms “increase”, “improve” or “enhance” in the context of a yield-related trait are interchangeable and shall mean in the sense of the application at least a 3%, 4%, 5%, 6%, 7%, 8%, 9% or 10%, preferably at least 15% or 20%, more preferably 25%, 30%, 35% or 40% increase in the yield-related trait(s) in comparison to control plants as defined herein.

### Marker Assisted Breeding

Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

### Use as Probes in (Gene Mapping)

Use of nucleic acids encoding the protein of interest for genetically and physically mapping the genes requires only a nucleic acid sequence of at least 15 nucleotides in length. These nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the nucleic acids encoding the protein of interest. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding the protein of interest in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes (or null control plants) are individuals missing the transgene by segregation. Further, control plants are grown under equal growing conditions to the growing conditions of the plants of the invention, i.e. in the vicinity of, and simultaneously with, the plants of the invention. A “control plant” as used herein refers not only to whole plants, but also to plant parts, including seeds and seed parts.

### Propagation Material

“Propagation material” is any kind of organ, tissue, or cell of a plant capable of developing into a complete plant. “Propagation material” can be based on vegetative reproduction (also known as vegetative propagation, vegetative multiplication, or vegetative cloning) or sexual reproduction. Propagation material can therefore be seeds or parts of the non-reproductive organs, like stem or leave. In particular, with respect to Poaceae, suitable propagation material can also be sections of the stem, i.e., stem cuttings (like setts).

### Stalk

A “stalk” is the stem of a Poaceae and is also known as the “millable cane” in particular for Saccharum species like sugarcane. In the context of Poaceae “stalk”, “stem”, “shoot”, or “tiller” are used interchangeably.

### Sett

A “sett” is a section of the stem of a Poaceae, in particular for Saccharum species like sugarcane, which is suitable to be used as propagation material. Synonymous expressions to “sett” are “seed-cane”, “stem cutting”, “section of the stalk”, and “seed piece”.

In the following, the expression “as defined in claim/item X” is meant to direct the artisan to apply the definition as disclosed in item/claim X. For example, “a nucleic acid as defined in item 1” has to be understood so that the definition of the nucleic acid as in item 1 is to be applied to the nucleic acid. In consequence the term “as defined in item” or “as defined in claim” may be replaced with the corresponding definition of that item or claim, respectively.

### DETAILED DESCRIPTION

The present invention shows that increasing expression in a plant of a flavodoxin nucleic acid encoding a flavodoxin polypeptide using a particular type of promoter and plastid targeting results in plants having one or more enhanced yield-related trait relative to control plants.

Any reference hereinafter to a “protein useful in the methods of the invention” is taken to mean a flavodoxin polypeptide as defined herein. Any reference hereinafter to a “nucleic acid useful in the methods of the invention” is taken to mean a nucleic acid capable of encoding such a flavodoxin polypeptide with plastid targeting. In one embodiment any reference to a protein or nucleic acid or expression construct “useful in the methods of the invention” is to be understood to mean proteins or nucleic acids or expression construct “useful in the methods, vector constructs, plants, harvestable parts and products of the invention”. The nucleic acid to be introduced into a plant (and therefore useful in performing the methods of the invention) is any nucleic acid encoding the type of protein which will now be described, hereafter also named “POI nucleic acid” or “POI gene” or “flavodoxin nucleic acid” or “flavodoxin nucleic acid” or “flavodoxin gene”, preferably encoding said protein with a targeting signal to the plastid of a plant.

Any reference herein to “a particular promoter” is taken to mean a PCPR promoter as defined herein.

Thus, a flavodoxin nucleic acid encoding a flavodoxin polypeptide is useful in the genetic constructs, methods, plants, harvestable parts and products of the present invention. Preferably, the flavodoxin nucleic acid is an isolated nucleic acid molecule comprising a nucleic acid selected from the group consisting of:
(i) a nucleic acid having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence represented by SEQ ID NO: 1, 13 or 15, or a functional fragment, derivative, orthologue, or paralogue thereof;
(ii) the complementary sequence of anyone of the nucleic acids of (i);
(iii) a nucleic acid encoding a flavodoxin polypeptide having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 2 or 16, or a functional fragment, derivative, orthologue, or paralogue thereof; preferably the flavodoxin polypeptide confers one or more enhanced yield-related traits relative to control plants; and
(iv) a nucleic acid molecule which hybridizes with a nucleic acid molecule of (i) to (iii) under stringent hybridization conditions.

More preferably, the isolated flavodoxin nucleic acid comprising a nucleic acid selected from the group consisting of:
(i) a nucleic acid having in increasing order of preference at least 90%, at least 91%, at least 92%, at least 93%, at least 94%, at least 95%, at least 96%, at least 97%, at least 98%, at least 99% or at least 100% sequence identity to the nucleic acid sequence represented by SEQ ID NO: 1, 13 or 15;
(ii) the complementary sequence of anyone of the nucleic acids of (i);
(iii) a nucleic acid encoding a flavodoxin polypeptide having in increasing order of preference at least 90%, at least 91%, at least 92%, at least 93%, at least 94%, at least 95%, at least 96%, at least 97%, at least 98%, at least 99% or at least 100% sequence identity to the amino acid sequence represented by SEQ ID NO: 2 or 16, preferably the flavodoxin polypeptide confers one or more enhanced yield-related traits relative to control plants; and
(iv) a nucleic acid molecule which hybridizes with a nucleic acid molecule of (i) to (iii) under high stringency hybridization conditions.

Percentages of identity of a nucleic acid are indicated with reference to the entire nucleotide region given in a sequence specifically disclosed herein.

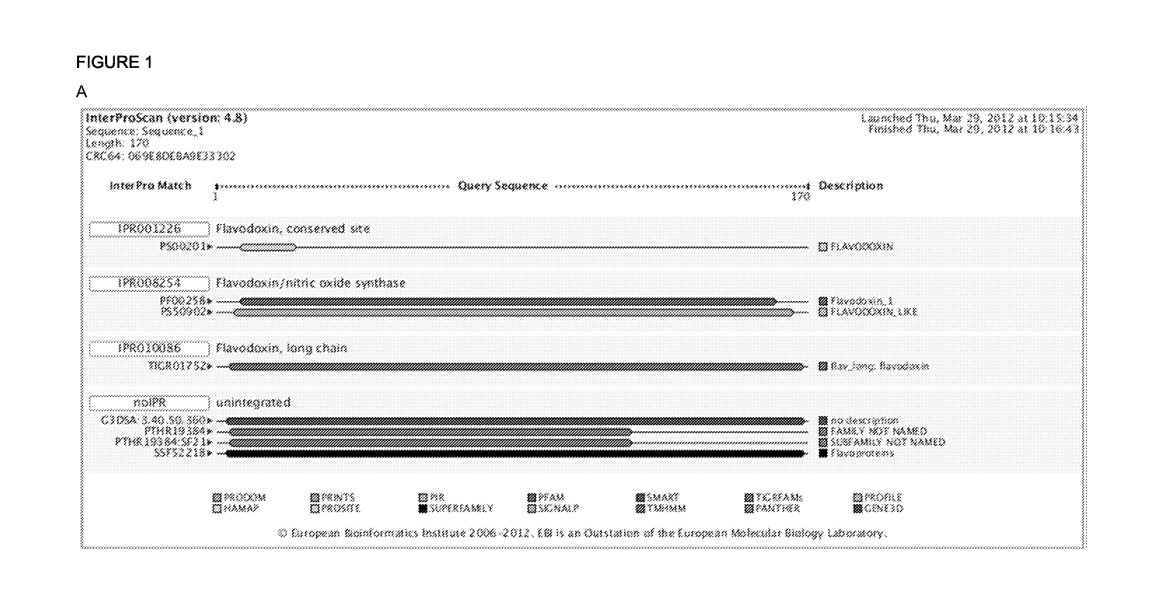

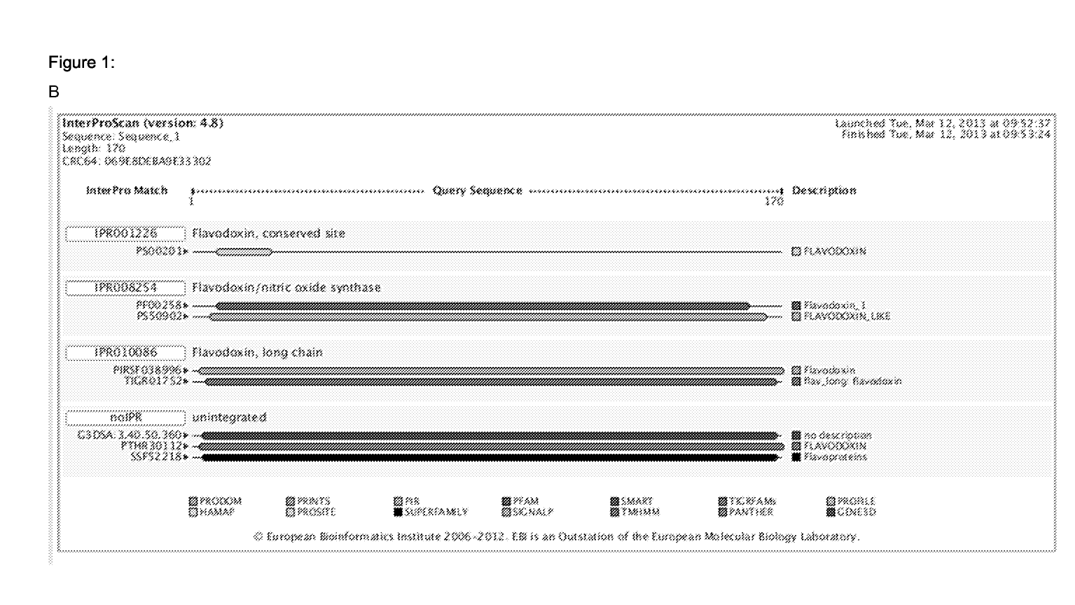

In a preferred embodiment the flavodoxin nucleic acid useful in the methods, vector constructs, plants, harvestable parts and products of the invention encodes a polypeptide comprising one or more of the domains and motifs listed in table B, more preferably the PFAM domain PF00258, preferably when analyzed with the InterproScan software as described in example 2. Further preferred is a localization and/or order of the one or more domains and/or motifs listed in table B within the polypeptide sequence of the flavodoxin polypeptide that is substantially the same as the one shown for SEQ ID NO: 2 in FIG. 1.

Most preferably, the isolated flavodoxin nucleic acid comprises or consists of a sequence as represented in SEQ ID NO: 1, 13 or 15, a complement thereof, a nucleic acid encoding a flavodoxin polypeptide with SEQ ID NO: 2 or 16, or a nucleic acid molecule which hybridizes with anyone of these nucleic acid molecules or a complementary sequence thereof under stringent hybridization conditions, and preferably encoding a polypeptide comprising one or more of the domains and motifs listed in table B, more preferably the PFAM domain PF00258, preferably when analyzed with the InterproScan software as described in example 2.

Preferred flavodoxin nucleic acids are referenced in Table 2 and/or the sequence listing. In one embodiment the flavodoxin nucleic acid comprises a nucleic acid sequence referenced in Table 2 and/or the sequence listing. More preferred as flavodoxin nucleic acid is a nucleic acid sequence comprising the flavodoxin gene of Anabaena sp., preferably Anabaena PCC7119, or Synechocystis sp., preferably Synechocystis sp. PCC 6803.

Most preferred as flavodoxin nucleic acid is a nucleic acid sequence comprising the flavodoxin gene of Anabaena sp., preferably Anabaena PCC7119.

In one embodiment the invention relates to the methods, vector constructs, plants, harvestable parts and products as described herein, comprising the codon optimised flavodoxin gene of Anabaena as disclosed in SEQ ID NO: 13 encoding the flavodoxin protein of SEQ ID NO: 2 or functional fragment, derivative, orthologue, or paralogue thereof as described herein, wherein said flavodoxin polypeptide, functional fragment, derivative, orthologue, or paralogue is linked to a transit peptide as described herein and functionally linked to a promoter suitable for expression in plants. Suitable promoters other than the promoter disclosed in SEQ ID NO: 7 are known in the art.

In one embodiment the invention relates to the methods, vector constructs, plants, harvestable parts and products as described herein, comprising the flavodoxin gene of Synechocystis sp. PCC 6803 as disclosed in SEQ ID NO: 15 or encoding the flavodoxin protein of SEQ ID NO: 16, or functional fragment, derivative, orthologue, or paralogue thereof as described herein, wherein said flavodoxin polypeptide, functional fragment, derivative, orthologue, or paralogue is linked to a transit peptide as described herein and functionally linked to a promoter suitable for expression in plants. Suitable promoters other than the promoter disclosed in SEQ ID NO: 7 are known in the art. The sequences of the polypeptides encoded are shown in SEQ ID NO: 16 & 18, with or without a pea FNR transit peptide, respectively.

Further nucleic acid variants useful in practising the methods of the invention include portions of nucleic acids encoding flavodoxin polypeptides, functional fragments of nucleic acids encoding flavodoxin polypeptides, nucleic acids hybridising to nucleic acids encoding flavodoxin polypeptides, splice variants of nucleic acids encoding flavodoxin polypeptides, allelic variants of nucleic acids encoding flavodoxin polypeptides and variants of nucleic acids encoding flavodoxin polypeptides obtained by gene shuffling. The terms hybridising sequence, splice variant, allelic variant and gene shuffling are as described herein.

Nucleic acids encoding flavodoxin polypeptides need not be full-length nucleic acids, since performance of the methods of the invention does not always rely on the use of full-length nucleic acid sequences. According to the present invention, there is provided a method for enhancing one or more yield-related traits in plants, comprising introducing and expressing in a plant a functional fragment of any one of the nucleic acid sequences given in Table 2 and/or the sequence listing, or a portion of a nucleic acid encoding an orthologue, paralogue or homologue of any of the amino acid sequences given in Table 2 and/or the sequence listing.

A fragment of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Fragments of a flavodoxin nucleic acid described herein encode a flavodoxin polypeptide as defined herein or at least a part thereof, which has substantially the same biological activity as the amino acid sequences given in Table 2 and/or the sequence listing. Preferably, the portion is a portion of any one of the nucleic acids given in Table 2 and/or the sequence listing, or is a portion of a nucleic acid encoding an orthologue or paralogue of any one of the amino acid sequences given in Table 2 and/or the sequence listing. Preferably the portion is at least about 100, at least about 200, at least about 300, at least about 400, at least about 500, at least about 600, at least about 700, at least about 800, at least about 900, at least about 1000 or more nucleotides, preferably consecutive nucleotides, preferably counted from the 5′ or 3′ end of the nucleic acid, in length of any of the nucleic acid sequences given in Table 2 and/or the sequence listing. Preferably, the flavodoxin nucleic acid comprises at least about 100, at least about 200, at least about 300, at least about 400, at least about 500 nucleotides, preferably consecutive nucleotides, preferably counted from the 5′ or 3′ end of the nucleic acid, or up to the full length of the nucleic acid sequence set out in SEQ ID NO: 1, 13 or 15.

Preferably the portion of the flavodoxin nucleic acid is about 400-425, about 425-450, about 450-475, about 475-500, about 500-525, about 525-550, about 550-575, about 575-600, about 625-650, about 650-675, about 675-700, about 700-725, about 725-750, about 750-775, about 775-800, about 800-825, about 825-850, about 850-875, about 875-900, about 925-950, about 950-975, about 975-1000 nucleotides, preferably consecutive nucleotides, preferably counted from the 5′ or 3′ end of the nucleic acid, in length, of the nucleic acid sequences given in Table 2 and/or the sequence listing. Preferably, the flavodoxin nucleic acid portion is about 400-425, about 425-450, about 450-475, about 475-500 nucleotides, preferably consecutive nucleotides, preferably counted from the 5′ or 3′ end of the nucleic acid, or up to the full length of the nucleic acid sequence set out in SEQ ID NO: 1, 13 or 15.

Another nucleic acid variant is a nucleic acid capable of hybridising, under reduced stringency conditions, preferably under stringent conditions, more preferably under high stringent conditions, with a nucleic acid encoding a flavodoxin polypeptide as defined herein, or with a portion as defined herein or a complement of either.

The hybridising sequence is capable of hybridising to the complement of anyone of the nucleic acids given in Table 2 and/or the sequence listing, or to a portion of any of these sequences, a portion being as defined herein, or the hybridising sequence is capable of hybridising to the complement of a nucleic acid encoding an orthologue or paralogue of any one of the nucleic acid sequences given in Table 2 and/or the sequence listing. Most preferably, the hybridising sequence is capable of hybridising to the complement of a nucleic acid given in SEQ ID NO: 1, 13 or 15 or to the complement of a nucleic acid encoding the polypeptide as represented by SEQ ID NO: 2 or 16 or to a portion thereof. In one embodiment, the hybridization conditions are of medium stringency, preferably of high stringency, as defined herein.

Preferably, the hybridising sequence encodes a polypeptide with an amino acid sequence which comprises SEQ ID NO: 2 or 16.

Preferred splice variants are splice variants of a nucleic acid represented by SEQ ID NO: 1, 13 or 15, or a splice variant of a nucleic acid encoding an orthologue or paralogue of SEQ ID NO: 2 or 16.

Furthermore, nucleic acid variants may also be obtained by site-directed mutagenesis. Several methods are available to achieve site-directed mutagenesis, the most common being PCR based methods (Current Protocols in Molecular Biology. Wiley Eds.). Flavodoxin polypeptides differing from the sequence of SEQ ID NO: 2 or 16 by one or several amino acids (substitution(s), insertion(s) and/or deletion(s) as defined herein) may equally be useful to increase the yield of plants in the methods and constructs and plants of the invention.

Nucleic acids encoding flavodoxin polypeptides may be derived from any natural or artificial source. The nucleic acid may be modified from its native form in composition and/or genomic environment through deliberate human manipulation. Preferably the flavodoxin polypeptide-encoding nucleic acid is from a bacterium, preferably a cyanobacterium, most preferably from Anabaena.

In another embodiment, the present invention extends to recombinant chromosomal DNA comprising a nucleic acid sequence (including a particular promoter employed) useful in the methods of the invention, wherein said nucleic acid is present in the chromosomal DNA as a result of recombinant methods, but is not in its natural genetic environment. In a further embodiment the recombinant chromosomal DNA of the invention is comprised in a plant cell. DNA comprised within a cell, particularly a cell with cell walls like a plant cell, is better protected from degradation or damage than a bare nucleic acid sequence. The same holds true for a DNA construct comprised in a host cell, for example a plant cell.

In a preferred embodiment the invention relates to compositions comprising the recombinant chromosomal DNA of the invention and/or the construct of the invention and a host cell, preferably a plant cell, wherein the recombinant chromosomal DNA and/or the construct are comprised within the host cell, preferably within a plant cell or a host cell with a cell wall. In a further embodiment said composition comprises dead host cells, living host cells or a mixture of dead and living host cells, wherein the recombinant chromosomal DNA and/or the construct of the invention may be located in dead host cells and/or living host cell. Optionally the composition may comprise further host cells that do not comprise the recombinant chromosomal DNA of the invention or the construct of the invention. The compositions of the invention may be used in processes of multiplying or distributing the recombinant chromosomal DNA and/or the construct of the invention, and or alternatively to protect the recombinant chromosomal DNA and/or the construct of the invention from breakdown and/or degradation as explained herein above. The recombinant chromosomal DNA of the invention and/or the construct of the invention can be used as a quality marker of the compositions of the invention, as an indicator of origin and/or as an indication of producer.

A flavodoxin polypeptide as described herein is useful in the genetic constructs, methods, plants, harvestable parts and products of the present invention. Preferably, the flavodoxin polypeptide is a bacterial flavodoxin polypeptide, for example a cyanobacterial flavodoxin polypeptide such as the flavodoxin of the cyanobacterium Anabaena PCC7119 (Fillat M. et al (1991) Biochem J. 280 187-191) or SEQ ID NO: 2 or the Synechocystis flavodoxin disclosed in SEQ ID NO: 16. Other suitable flavodoxin polypeptides include flavodoxins from photosynthetic anoxigenic bacteria, enterobacteria, diazotrophs and algae. Examples of nucleic acids encoding flavodoxin polypeptides suitable for use according to the present invention are exemplified in Table 2 and/or the sequence listing. Whilst a wild type flavodoxin polypeptide is preferred, a flavodoxin polypeptide may also be a fragment, mutant, derivative, variant or allele of such a wild type sequence.

Suitable fragments, mutants, derivatives, variants and alleles are those which retain the functional characteristics of the polypeptide encoded by the wild-type flavoprotein gene, especially the ability to act as an anti-oxidant. Changes to a sequence, to produce a mutant, variant or derivative, may be by one or more of addition, insertion, deletion or substitution of one or more nucleotides in the nucleic acid, leading to the addition, insertion, deletion or substitution of one or more amino acids in the encoded polypeptide. Of course, changes to the nucleic acid which make no difference to the encoded amino acid sequence are included.

A polypeptide which is a member of the flavodoxin family or which is an amino acid sequence variant, allele, derivative or mutant thereof may comprise an amino acid sequence which shares greater than about 30% sequence identity, greater than about 35%, greater than about 40%, greater than about 45%, greater than about 55%, greater than about 65%, greater than about 70%, greater than about 80%, greater than about 90% or greater than about 95%, preferably greater than about 96%, greater than about 97%, greater than about 98%, or greater than about 99% sequence identity with a flavodoxin polypeptide encoded by a flavodoxin nucleic acid as shown in Table 2 and/or the sequence listing.

A polypeptide which is a member of the Flavodoxin family or which is an amino acid sequence variant, allele, derivative or mutant thereof may comprise an amino acid sequence which shares greater than about 30% sequence identity, greater than about 35%, greater than about 40%, greater than about 45%, greater than about 55%, greater than about 65%, greater than about 70%, greater than about 80%, greater than about 90% or greater than about 95%, preferably greater than about 96%, greater than about 97%, greater than about 98%, or greater than about 99% sequence identity with the amino acid sequence of Anabaena PCC7119 flavodoxin.

In certain embodiments, a flavodoxin polypeptide may show little overall homology, say about 20%, or about 25%, or about 30%, or about 35%, or about 40% or about 45%, with the Anabaena PCC7119 flavodoxin sequence (SEQ ID NO: 2) or the Synechocystis flavodoxin (SEQ ID NO: 16), even though it possesses substantially the same anti-oxidation activity. However, in functionally significant domains or regions, the amino acid homology may be much higher. For example, a flavodoxin polypeptide comprises an FMN-binding domain which has high homology to the flavodoxin FMN binding domain (a flavodoxin-like domain). Putative functionally significant domains or regions can be identified using processes of bioinformatics, including comparison of the sequences of homologues.

In a preferred embodiment the flavodoxin polypeptide useful in the methods, plants, harvestable parts and products of the invention is a polypeptide comprising one or more of the domains and motifs listed in table B, more preferably the PFAM domain PF00258, preferably when analyzed with the InterproScan software as described in example 2. Further preferred is a localization and/or order of the one or more domains and/or motifs listed in table B within the polypeptide sequence of the flavodoxin polypeptide that is substantially the same as the one shown for SEQ ID NO: 2 in FIG. 1.

Most preferred as flavodoxin polypeptide is a polypeptide comprising or consisting of the flavodoxin protein encoded by any of the nucleic acid sequences given in Table 2 and/or the sequence listing, preferably of Anabaena sp., preferably Anabaena PCC7119, or Synechocystis sp., preferably Synechocystis sp. PCC 6803, more preferably the polypeptide of SEQ ID NO: 2 or 16 encoded by the nucleic acid as disclosed in SEQ ID NO: 1, 13 or 15, respectively, and most preferably the polypeptide of SEQ ID NO: 2.

Preferably, the flavodoxin polypeptide is a polypeptide comprising a polypeptide selected from the group consisting of:
(i) a polypeptide having in increasing order of preference at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 2 or 16, or a functional fragment, derivative, orthologue, or paralogue thereof; preferably the flavodoxin polypeptide confers one or more enhanced yield-related traits relative to control plants;
(ii) a polypeptide encoded by a nucleic acid having in increasing order of preference at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence represented by SEQ ID NO: 1, 13 or 15, or a functional fragment, derivative, orthologue, or paralogue thereof; preferably the flavodoxin polypeptide confers one or more enhanced yield-related traits relative to control plants.

More preferably, the flavodoxin polypeptide is a polypeptide comprising a polypeptide selected from the group consisting of:
(i) a polypeptide having in increasing order of preference at least 90%, at least 91%, at least 92%, at least 93%, at least 94%, at least 95%, at least 96%, at least 97%, at least 98%, at least 99% or at least 100% sequence identity to the amino acid sequence represented by SEQ ID NO: 2 or 16, or a functional fragment, derivative, orthologue, or paralogue thereof;
(ii) a polypeptide encoded by a nucleic acid having in increasing order of preference at least 90%, at least 91%, at least 92%, at least 93%, at least 94%, at least 95%, at least 96%, at least 97%, at least 98%, at least 99% or at least 100% sequence identity to the nucleic acid sequence represented by SEQ ID NO: 1, 13 or 15, or a fragment, derivative, orthologue, or paralogue thereof. Preferably the flavodoxin polypeptide confers one or more enhanced yield-related traits relative to control plants, preferably control plants not expressing the flavodoxin polypeptide.

Percentages of identity of a polypeptide or protein are indicated with reference to the entire amino acid sequence specifically disclosed herein.

Preferably, the flavodoxin polypeptide comprises at least about 50, at least about 75, at least about 100, at least about 110, at least about 120, at least about 130, at least about 140, at least about 145, at least about 150, at least about 155, at least about 160, at least about 165, or at least about 167 amino acids, preferably consecutive amino acids, preferably counted from the N-terminus or C-terminus of the amino acid sequence, or up to the full length of the amino acid sequence set out in SEQ ID NO: 2 or 16. Preferably, the flavodoxin polypeptide has substantially the same biological activity as SEQ ID NO: 2 or 16. Preferably the flavodoxin polypeptide confers one or more enhanced yield-related traits relative to control plants, preferably control plants not expressing the flavodoxin polypeptide.

Preferably, the flavodoxin polypeptide comprises at least about 50, at least about 75, at least about 100, at least about 110, at least about 120, at least about 130, at least about 140, at least about 145, at least about 150, at least about 155, at least about 160, at least about 165, or at least about 167 amino acids, preferably consecutive amino acids, preferably counted from the N-terminus or C-terminus of the amino acid sequence, or up to the full length of any of the amino acid sequences encoded by the nucleic acid sequences set out in Table 2 and/or the sequence listing. Preferably, the flavodoxin polypeptide has substantially the same biological activity as the respective sequence of Table 2 and/or the sequence listing. Preferably the flavodoxin polypeptide confers one or more enhanced yield-related traits relative to control plants, preferably control plants not expressing the flavodoxin polypeptide.

Preferably, the flavodoxin polypeptide comprises about 50-75, about 75-100, about 100-110, about 110-120, about 120-130, about 130-140, about 140-150, about 150-160, about 160-170 amino acids, preferably consecutive amino acids, preferably counted from the N-terminus or C-terminus of the amino acid sequence, or up to the full length of any of the amino acid sequences encoded by the nucleic acid sequences set out in Table 2 and/or the sequence listing. Preferably, the flavodoxin polypeptide has substantially the same biological activity as the respective sequence of Table 2 and/or the sequence listing. Preferably the flavodoxin polypeptide confers one or more enhanced yield-related traits relative to control plants, preferably control plants not expressing the flavodoxin polypeptide.

Preferably, the flavodoxin polypeptide comprises about 50-75, about 75-100, about 100-110, about 110-120, about 120-130, about 130-140, about 140-150, about 150-160, about 160-170 amino acids, preferably consecutive amino acids, preferably counted from the N-terminus or C-terminus of the amino acid sequence, or up to the full length of the amino acid sequence set out in SEQ ID NO: 2 or 16. Preferably, the flavodoxin polypeptide has substantially the same biological activity as SEQ ID NO: 2 or 16. Preferably the flavodoxin polypeptide confers one or more enhanced yield-related traits relative to control plants, preferably control plants not expressing the flavodoxin polypeptide.

More preferably, the isolated flavodoxin polypeptide comprises or consists of SEQ ID NO: 2 or 16, or is encoded by a nucleic acid with SEQ ID NO: 1, 13 or 15, preferably the flavodoxin polypeptide confers one or more enhanced yield-related traits relative to control plants.

The polypeptides encoded by allelic variants useful in the methods of the present invention have substantially the same biological activity as the flavodoxin polypeptide of SEQ ID NO: 2 or 16 and any of the amino acid sequences encoded by the nucleic acid sequences depicted in Table 2 and/or the sequence listing. Allelic variants exist in nature, and encompassed within the methods of the present invention is the use of these natural alleles. Preferably, the allelic variant is an allelic variant of SEQ ID NO: 1, 13 or 15 or an allelic variant of a nucleic acid encoding an orthologue or paralogue of SEQ ID NO: 2 or 16.

In another embodiment the polypeptide sequences useful in the methods, constructs, plants, harvestable parts and products of the invention have substitutions, deletions and/or insertions compared to the sequence of SEQ ID NO: 2 or 16, wherein the amino acid substitutions, insertions and/or deletions may range from 1 to 10 amino acids each.

The invention also provides genetic constructs, like expression constructs or expressions cassettes, or vector constructs, comprising a flavodoxin nucleic acid. Preferably, these genetic constructs are suitable for the introduction and/or expression in plants, plant parts or plant cells of nucleic acids encoding flavodoxin polypeptides. The expression constructs may be inserted into vectors constructs, which may be commercially available, suitable for transforming into plants or host cells and suitable for expression of the gene of interest in the transformed cells. The invention also provides use of a genetic construct as defined herein in the methods of the invention. Thus, another embodiment of the present invention is an expression construct or expression cassette comprising a flavodoxin nucleic acid.

The genetic constructs of the invention may be comprised in a host cell, plant cell, seed, agricultural product or plant or plant part. Plants or host cells are transformed with a genetic construct such as a vector construct or an expression construct comprising any of the flavodoxin nucleic acids described herein.

In one embodiment the genetic construct of the invention confers increased yield or yield-related traits(s) to a plant when it has been introduced into said plant, which plant expresses the nucleic acid encoding the flavodoxin polypeptide comprised in the genetic construct. In another embodiment the genetic construct of the invention confers increased yield or yield-related traits(s) to a plant comprising plant cells in which the construct has been introduced, which plant cells express the nucleic acid encoding the flavodoxin polypeptide comprised in the genetic construct.

The skilled artisan is well aware of the genetic elements that must be present in the genetic construct in order to successfully transform, select and propagate host cells containing the sequence of interest.

More specifically, the present invention provides an expression construct comprising:
(a) a flavodoxin nucleic acid encoding a flavodoxin polypeptide as defined above;
(b) one or more control sequences capable of driving expression of the nucleic acid sequence of (a), wherein the control sequence is preferably a promoter sequence; and optionally
(c) a transcription termination sequence.

Most preferably, the present invention provides an expression construct comprising:
(a) a flavodoxin nucleic acid encoding a flavodoxin polypeptide as defined above;
(b) a transit nucleic acid sequence encoding a transit peptide;
(c) a promoter sequence, operably linked to the nucleic acid of (a) and (b), wherein the promoter sequence comprises the PCPR promoter (protochlorophyllide reductase promoter), or a functional fragment or variant or homologue, orthologue or paralogue thereof; and optionally
(d) a transcription termination sequence.

In a preferred embodiment any reference to a PCPR promoter or a protochlorophyllide reductase promoter throughout this application is to be understood to refer to a promoter that in its natural genetic context controls the expression of a nucleic acid encoding a protochlorophyllide reductase. Preferably said promoter is from a dicot or a monocot plant, more preferably from a Poaceae, even more preferably from rice and most preferably the promoter with a sequence as disclosed in SEQ ID NO: 7.

Preferably, the flavodoxin nucleic acid of the expression construct comprises any of the flavodoxin nucleic acids described herein, preferably, as set out in Table 2 and/or the sequence listing, or a functional fragment or variant or homologue, orthologue or paralogue thereof. Preferably, the transit nucleic acid is selected from the nucleic acid sequences encoding any of the transit peptides described herein, preferably, as set out in Table 3, or a functional fragment or variant or homologue, orthologue or paralogue thereof.

Preferably, the promoter sequence comprises a PCPR promoter sequence as described herein, preferably the rice PCPR promoter (protochlorophyllide reductase promoter), or a functional fragment or variant or homologue, orthologue or paralogue thereof.

Preferably, the flavodoxin nucleic acid of the expression construct comprises a nucleic acid selected from the group consisting of:
(i) a nucleic acid having in increasing order of preference at least 80%, at least 85%, at least 90%, at least 93%, at least 94%, at least 95%, at least 96%, at least 97%, at least 98%, at least 99% or at least 100% sequence identity to the nucleic acid sequence represented by SEQ ID NO: 1, 13 or 15, wherein the nucleic acid preferably has the same biological activity as SEQ ID NO: 2 or 16, preferably, wherein the nucleic acid encodes a flavodoxin polypeptide that confers one or more enhanced yield-related traits relative to control plants;
(ii) the complementary sequence of anyone of the nucleic acids of (i);
(iii) a nucleic acid encoding a flavodoxin polypeptide having in increasing order of preference at least 80%, at least 85%, at least 90%, at least 93%, at least 94%, at least 95%, at least 96%, at least 97%, at least 98%, at least 99% or at least 100% sequence identity to the amino acid sequence represented by SEQ ID NO: 2 or 16, preferably the flavodoxin polypeptide confers one or more enhanced yield-related traits relative to control plants; and
(iv) a nucleic acid molecule which hybridizes with a nucleic acid molecule of (i) to (iii) under stringent hybridization conditions, wherein the nucleic acid preferably has substantially the same biological activity as SEQ ID NO: 2 or 16 or a complementary sequence thereof, preferably, wherein the nucleic acid encodes a flavodoxin polypeptide that confers one or more enhanced yield-related traits relative to control plants

Most preferably, the flavodoxin nucleic acid of the expression construct comprises or consists of SEQ ID NO: 1, 13 or 15, a complement thereof, a nucleic acid encoding a flavodoxin polypeptide with SEQ ID NO: 2 or 16, or a nucleic acid molecule which hybridizes with anyone of these nucleic acid molecules under stringent hybridization conditions.

Yet another embodiment relates to genetic constructs useful in the methods, vector constructs, plants, harvestable parts and products of the invention wherein the genetic construct comprises the flavodoxin nucleic acid of the invention functionally linked a promoter as disclosed herein above and further functionally linked to one or more of
1) nucleic acid expression enhancing nucleic acids (NEENAs):
   a) as disclosed in the international patent application published as WO2011/023537 in table 1 on page 27 to page 28 and/or SEQ ID NO: 1 to 19 and/or as defined in items i) to vi) of claim 1 of said international application which NEENAs are herewith incorporated by reference; and/or
   b) as disclosed in the international patent application published as WO2011/023539 in table 1 on page 27 and/or SEQ ID NO: 1 to 19 and/or as defined in items i) to vi) of claim 1 of said international application which NEENAs are herewith incorporated by reference; and/or
   c) as contained in or disclosed in:
      i) the European priority application filed on 5 Jul. 2011 as EP 11172672.5 in table 1 on page 27 and/or SEQ ID NO: 1 to 14937, preferably SEQ ID NO: 1 to 5, 14936 or 14937, and/or as defined in items i) to v) of claim 1 of said European priority application which NEENAs are herewith incorporated by reference; and/or
      ii) the European priority application filed on 6 Jul. 2011 as EP 11172825.9 in table 1 on page 27 and/or SEQ ID NO: 1 to 65560, preferably SEQ ID NO: 1 to 3, and/or as defined in items i) to v) of claim 1 of said European priority application which NEENAs are herewith incorporated by reference; and/or
   d) equivalents having substantially the same enhancing effect; and/or
2) functionally linked to one or more Reliability Enhancing Nucleic Acid (RENA) molecule
   a) as contained in or disclosed in the European priority application filed on 15 Sep. 2011 as EP 11181420.8 in table 1 on page 26 and/or SEQ ID NO: 1 to 16 or 94 to 116666, preferably SEQ ID NO: 1 to 16, and/or as defined in point i) to v) of item a) of claim 1 of said European priority application which RENA molecule(s) are herewith incorporated by reference; or
   b) equivalents having substantially the same enhancing effect.

A preferred embodiment of the invention relates to a genetic construct useful in the methods, vector constructs, plants, harvestable parts and products of the invention and comprising a nucleic acid encoding a flavodoxin polypeptide of the invention under the control of a promoter as described herein above, wherein the NEENA, RENA and/or the promoter is heterologous to the flavodoxin nucleic acid molecule of the invention.

The genetic constructs—like expression constructs—described herein and the vector constructs described herein are useful in the methods, plants, harvestable parts and products of the invention. Preferably they confer an increase of one or more yield-related traits when stably introduced into a plant as described herein. Preferably plants carrying the construct of the invention show an increase in one or more yield-related traits grown udner non-stress conditions, drought conditions or conditions of nitrogen deficiency, more preferably under non-stress conditions.

The promoter in a genetic construct described herein may be a native or may be a non-native promoter to the nucleic acid described herein, i.e., a promoter not regulating the expression of said nucleic acid in its natural genetic environment.

In one embodiment the PCPR promoter (protochlorophyllide reductase promoter) in a genetic construct described herein is a promoter active in green tissue(s), leaves and/or stems, preferably a green tissue-specific promoter with substantially the same temporal and/or spatial expression pattern and/or substantially the same expression strength as the promoter shown in SEQ ID NO: 7, and preferably is of plant origin or synthetic. Advantageously, the green-tissue specific PCPR promoter is resulting in a stronger increase of one or more desired yield-related traits as any other promoter, whether natural or synthetic, such as constitutive or ubiquitous promoter, developmentally-regulated promoter, inducible promoter, organ-specific or tissue-specific promoter, for example a root-specific promoter, seed-specific promoter, endosperm-specific promoters, embryo specific promoters, embryo specific promoters, aleurone-specific promoters, green tissue-specific promoter, stemspecific, leave-specific or meristem-specific promoter. Preferably, the promoter sequence operably linked to the nucleic acid encoding a transit peptide and a flavodoxin as defined herein comprises a PCPR promoter—more preferably the PCPR promoter from rice and even more preferably the PCPR promoter as disclosed in EP1532257 as “PRO123” with SEQ ID NO: 14, or as disclosed in SEQ ID NO: 7 of the present application—, or a functional fragment or variant or homologue, orthologue or paralogue thereof. More preferably, the promoter sequence consists of the PCPR promoter)—more preferably the PCPR promoter from rice and even more preferably the PCPR promoter as disclosed in EP1532257 as “PRO123” with SEQ ID NO: 14, or as disclosed in SEQ ID NO: 7 of the present application—, or a functional fragment or variant or homologue, orthologue or paralogue thereof. In one embodiment preferred promoter functional fragments or variants have in increasing order of preference at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even 100% sequence identity with the nucleic acid sequence represented by SEQ ID NO: 7.

Preferably, the portion of the promoter sequence is a functional portion of SEQ ID NO: 7. Preferably the portion is at least about 400, at least about 500, at least about 600, at least about 700, at least about 800, at least about 900, at least about 1000, at least about 1100 or more nucleotides, preferably consecutive nucleotides, preferably counted from the 5′ or 3′ end of the nucleic acid, in length, of the nucleic acid sequences given in SEQ ID NO: 7.

Preferably the portion of the promoter sequence is about 400-425, about 425-450, about 450-475, about 475-500, about 500-525, about 525-550, about 550-575, about 575-600, about 625-650, about 650-675, about 675-700, about 700-725, about 725-750, about 750-775, about 775-800, about 800-825, about 825-850, about 850-875, about 875-900, about 925-950, about 950-975, about 975-1000, about 1000-1025, about 1025-1100, about 1100-1125, about 1125-1150, about 1150-1175, about 1170-1179 nucleotides, preferably consecutive nucleotides, preferably counted from the 5′ or 3′ end of the nucleic acid, in length, of the nucleic acid sequences given in SEQ ID NO: 7.

Preferred promoter sequence comprises or consists of SEQ ID NO: 7.

The transit peptide encoded by the transit nucleic acid is preferably about 5, 10, 15, 16, 17, 18, 19, 20, 21, 22, 23, 24, 25, 26, 27, 28, 29, 30, 31, 32, 33, 34, 35, 36, 37. 38, 39, 40, 41, 42, 43, 44, 45, 46, 47, 48, 49, 50, 51, 52, 53, 54, or more amino acids long. Preferably the transit peptide directs the transport of a protein to other organelles within the cell. Preferably, the transit peptide targets the flavodoxin polypeptide to the nucleus, mitochondria, mitochondrial matrix, endoplasmic reticulum, chloroplasts, apicoplasts, chromoplast, cyanelle, thylakoid, amyloplast, peroxisome, glyoxysome, and/or hydrogenosome. Most preferably, the transit peptide targets the flavodoxin polypeptide to a plastid, preferably to a chloroplast. Preferably the transit peptide is cleaved from the polypeptide, preferably by a signal peptidase, after the polypeptide is transported. In another embodiment, the transit peptide is not cleaved from the polypeptide after the polypeptide is transported.

A chloroplast transit peptide suitable for use in accordance with certain embodiments of the present invention may be any peptide sequence which directs a polypeptide to the chloroplast of a plant cell. Suitable peptides may readily be identified by a skilled person and some examples are shown in Table 3. Other examples are known in the art.

In some preferred embodiments, a transit peptide may comprise or consist of the chloroplast transit peptide of the FAD-containing ferredoxin-NADP+ reductase (FNR), more preferably of the FNR of pea or Cyanophora paradoxa, which transit peptide even more preferably has the sequence shown in SEQ ID NO: 4 or 10, respectively. Its coding sequence is preferably as shown in SEQ ID NO: 3, or 8 or 9, respectively.

A nucleic acid encoding any flavodoxin polypeptide as defined above may be used in accordance with the present invention with any suitable chloroplast transit peptide as defined above. Preferably, the flavodoxin polypeptide is not fused to a transit peptide with which it is naturally associated, i.e., it is fused to a heterogeneous transit peptide. Flavodoxin polypeptides, which are not found in plants, are not naturally associated with chloroplast transit peptides.

A preferred transit nucleic acid sequence coding for a transit peptide is given in SEQ ID NO: 3, 8 or 9. Preferably, the transit nucleic acid sequence comprises or consists of a transit nucleic acid sequence as given in SEQ ID NO: 3, 8 or 9, or functional fragments or variants thereof. Preferred functional transit nucleic acid sequence fragments or variants have in increasing order of preference at least 50%, 51%, 52%, 53%, 54%, 55%, 56%, 57%, 58%_{,} 59%, 60%, 81%^{,} 62%, 63%, 64%, 65%, 66%, 67%, 68%, 69%, 70%, 71%, 72%, 73%, 74%_{,} 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence represented by SEQ ID NO: 3, 8 or 9 or any of the nucleic acid sequences coding for the transit peptides shown in Table 3.

In one embodiment the transit peptide differs from any transit peptide naturally linked to the flavodoxin protein(s) of table 2 and/or the sequence listing.

Preferably the portion of the transit nucleic acid sequence is at least about 15, at least about 30, at least about 45, at least about 60, at least about 75, at least about 90, at least about 120, at least about 135, at least about 150 or more nucleotides, preferably consecutive nucleotides, preferably counted from the 5′ end of the nucleic acid, in length of any of the nucleic acid sequences given in SEQ ID NO: 3, 8 or 9.

Preferably the portion of the transit nucleic acid sequence is 15 to 45, about 24 to 60, about 60-75, about 75-102, about 102-126, about 126-150 nucleotides, preferably consecutive nucleotides, preferably counted from the 5′ end of the nucleic acid, in length, of the nucleic acid sequences given in SEQ ID NO: 3, 8 or 9.

A preferred transit peptide is given in SEQ ID NO: 4 or 10. Preferably, the transit peptide comprises or consists of a transit peptide as given in SEQ ID NO: 4 or 10, or functional fragments or variants thereof. Preferred functional transit peptide fragments or variants have in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NOs: 4 or 10, or any of the transit peptides shown in Table 3.

Preferably, the transit peptide comprises at least about 5, at least about 10, at least about 15, at least about 20, at least about 25, at least about 30, at least about 35, at least about 40, at least about 45 or at least about 50 amino acids, preferably consecutive amino acids, preferably counted from the N-terminus of the amino acid sequence, or up to the full length of the amino acid sequence set out in SEQ ID NO: 4 or 10.

Preferably, the transit peptide comprises at least about 5, at least about 10, at least about 15, at least about 20, at least about 25, at least about 30, at least about 35, at least about 40, at least about 45, or at least about 50 amino acids, preferably consecutive amino acids, preferably counted from the N-terminus or C-terminus, preferably from the N-terminus of the amino acid sequence, or up to the full length of any of the amino acid sequence set out in Table 3.

Preferably, the transit peptide comprises about 5 to 20, about 20-25, about 25-30, about 30-35, about 35-40, about 40-45, about 45-50 amino acids, preferably consecutive amino acids, preferably counted from the N-terminus of the amino acid sequence, or up to the full length of the amino acid sequence set out in SEQ ID NO: 4 or 10.

Preferably, the transit peptide comprises about 5 to 20, about 20-25, about 25-30, about 30-35, about 35-40, about 40-45, about 45-50 amino acids, preferably consecutive amino acids, preferably counted from the N-terminus or C-terminus, preferably from the N-terminus of the amino acid sequence, or up to the full length of any of the amino acid sequence set out in Table 3.

Additional preferred chloroplast transit peptides are referenced in Table 3.

Preferably the expression construct comprises a nucleic acid selected from the group consisting of:
(i) a nucleic acid having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence represented by SEQ ID NO: 5, 12, 14 or 17, or a functional fragment, derivative, orthologue, or paralogue thereof;
(ii) a nucleic acid encoding an amino acid sequence in increasing order of preference with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 6, 11 or 18, or a functional fragment, derivative, orthologue, or paralogue thereof; and/or
(iii) the complementary sequence of anyone of the nucleic acids of (i) or (ii); and optionally a promoter sequence as described herein.

Preferably the functional portion of the nucleic acid encoding a flavodoxin polypeptide and a transit peptide is at least about 100, at least about 200, at least about 300, at least about 400, at least about 500, at least about 600, or more nucleotides, preferably consecutive nucleotides, preferably counted from the 5′ or 3′ end, preferably from the 5′ end of the nucleic acid, in length of any of the nucleic acid sequences given in SEQ ID NO: 5, 12, 14 or 17.

Preferably the functional portion of the nucleic acid encoding a flavodoxin polypeptide and a transit peptide is about 400-425, about 425-450, about 450-475, about 475-500, about 500-525, about 525-550, about 550-575, about 575-600, about 625-650, about 650-675 nucleotides, preferably consecutive nucleotides, preferably counted from the 5′ or 3′ end, preferably from the 5′ end of the nucleic acid, in length, of the nucleic acid sequences given in SEQ ID NO: 5, 12, 14 or 17.

More preferably, the expression construct comprises a nucleic acid sequence as set out in SEQ ID NO: 5, 12, 14 or 17.

Further preferred is an expression construct comprising a nucleic acid sequence coding for a polypeptide comprising a flavodoxin polypeptide and a transit sequence comprising an amino acid sequence in increasing order of preference with at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 6, or a functional fragment, derivative, orthologue, or paralogue thereof.

Preferably, the polypeptide comprising a flavodoxin polypeptide and a transit sequence comprises at least about 140, at least about 150, at least about 160, at least about 170, at least about 180, at least about 190, at least about 200, at least about 210, at least about 220 amino acids, preferably consecutive amino acids, preferably counted from the N-terminus or C-terminus, preferably from the N-terminus of the amino acid sequence, or up to the full length of the amino acid sequence set out in SEQ ID NO: 6.

Preferably, the flavodoxin polypeptide comprises about 100-110, about 110-120, about 120-130, about 130-140, about 140-150, about 150-160, about 160-170, about 170-180, about 180-190, about 190-200, about 200-210, about 210-220 amino acids, preferably consecutive amino acids, preferably counted from the N-terminus or C-terminus, preferably from the N-terminus of the amino acid sequence, or up to the full length of the amino acid sequence set out in SEQ ID NO: 6.

Thus, a further embodiment is a flavodoxin polypeptide encoded by an expression construct comprising:
(a) a flavodoxin nucleic acid encoding a flavodoxin polypeptide as described herein; and
(b) a transit nucleic acid sequence encoding a transit peptide as described herein; wherein the expression construct comprises a promoter sequence in functional linkage to the nucleic acid sequence comprising the flavodoxin nucleic acid sequence and the transit nucleic acid sequence and wherein the promoter sequence comprises the PCPR promoter (protochlorophyllide reductase promoter), preferably, or a functional fragment or variant or homologue, orthologue or paralogue thereof.

Preferably the polypeptide comprising a flavodoxin polypeptide and a transit sequence comprises a transit peptide from pea FAD-containing ferredoxin-NADP+ reductase (FNR) and a flavodoxin protein from Anabaena sp. (PCC7119).

Preferably, the fusion polypeptide comprising a flavodoxin polypeptide and a transit sequence comprises an amino acid sequence in increasing order of preference with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%_{, 75}%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 6, or a functional fragment, derivative, orthologue, or paralogue thereof.

More preferably, the polypeptide comprising a flavodoxin polypeptide and a transit sequence comprises or consists of an amino acid sequence as set out in SEQ ID NO: 6.

In some preferred embodiments, a fusion polypeptide comprising a flavodoxin polypeptide and a chloroplast targeting peptide preferably comprise or consists of the sequence shown in SEQ ID NO: 6. A suitable nucleic acid molecule encoding such a fusion polypeptide preferably comprise or consists of the sequence shown in SEQ ID NO: 5, 12, 14 or 17.

Preferably the expression construct comprises a nucleic acid encoding a transit peptide from pea FAD-containing ferredoxin-NADP+ reductase (FNR) and a flavodoxin protein from Anabaena sp. (PCC7119) and a PCPR promoter (protochlorophyllide reductase promoter), preferably the promoter sequence comprises or consists of the nucleotide sequences depicted in SEQ ID NO: 7.

Preferably the expression construct comprises a nucleic acid selected from the group consisting of:
(i) a nucleic acid having in increasing order of preference at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the nucleic acid sequence represented by SEQ ID NO: 5, 12, 14 or 17, or a fragment, derivative, orthologue, or paralogue thereof;
(ii) a nucleic acid sequence coding for a polypeptide comprising a flavodoxin polypeptide and a transit sequence comprising an amino acid sequence in increasing order of preference with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the amino acid sequence represented by SEQ ID NO: 6, or a fragment, derivative, orthologue, or paralogue thereof; and
(iii) the complementary sequence of anyone of the nucleic acids of (i) or (ii); and operatively linked thereto a promoter sequence of comprising in increasing order of preference at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even 100% sequence identity with the nucleic acid sequence represented by SEQ ID NO: 7.

Preferably, the expression construct comprises a nucleic acid encoding for a fusion protein comprising a transit peptide and a flavodoxin polypeptide as depicted in SEQ ID NO: 5, 12, 14 or 17 and operably linked thereto a promoter sequence as shown in SEQ ID NO: 7.

Optionally, one or more transcription termination sequences may be used in the construct introduced into a plant. Those skilled in the art will be aware of terminator sequences that may be suitable for use in performing the invention. Preferably, the construct comprises an expression cassette comprising a promoter sequence operably linked to the nucleic acid encoding a transit peptide and a flavodoxin polypeptide and a transcription termination sequence. Preferably the transcription termination sequence is a zein terminator (t-zein) linked to the 3′ end of the flavodoxin coding sequence. Most preferably, the expression cassette comprises a sequence having in increasing order of preference at least 95%, at least 96%, at least 97%, at least 98%, at least 99% identity to the sequence of the zein terminator (t-zein).

The genetic construct, vector construct, or expression construct described herein can further comprise one or more sequences encoding a selectable marker.

Preferred selectable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example 3-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that in attempts to stable or transient integrate nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described herein) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

A further embodiment of the present invention is a vector construct comprising a flavodoxin nucleic acid, an expression construct or expression cassette containing the flavodoxin nucleic acid as described herein.

A preferred embodiment is a recombinant vector construct comprising a nucleic acid sequence coding for a transit sequence as described herein (preferably selected from Table 3) and a flavodoxin polypeptide as described herein (the coding sequence preferably selected from Table 2 and/or the sequence listing) and, operably linked thereto, a promoter sequence as described herein (preferably as depicted in SEQ ID NO: 7), wherein the promoter sequence comprises the PCPR promoter (protochlorophyllide reductase promoter), or a functional fragment or variant or homologue, orthologue or paralogue thereof.

A further preferred embodiment is a recombinant vector construct comprising:
(a) (i) a flavodoxin nucleic acid having at least 60% identity, preferably at least 70% sequence identity, at least 80%, at least 90%, at least 95%, at least 98%, at least 99% sequence identity, or even 100% sequence identity with SEQ ID NO: 1, 13 or 15 or a functional fragment thereof, or an orthologue or a paralogue thereof;
   (ii) a nucleic acid coding for a flavodoxin protein having at least 60% identity, preferably at least 70% sequence identity, at least 80%, at least 90%, at least 95%, at least 98%, at least 99% sequence identity, or even 100% sequence identity with SEQ ID NO: 2 or 16, a functional fragment thereof, an orthologue or a paralogue thereof; and/or
   (iii) a nucleic acid capable of hybridizing under stringent conditions with any of the nucleic acids according to (i) or (ii) or a complementary sequence thereof;
      operably linked with
(b) a promoter sequence, wherein the promoter sequence preferably, comprises the PCPR promoter (protochlorophyllide reductase promoter), preferably, or a functional fragment or variant or homologue, orthologue or paralogue thereof; and preferably
(c) a transcription termination sequence.

Furthermore, a recombinant vector construct is provided comprising:
(a) (i) a flavodoxin nucleic acid having at least 95%, at least 98%, at least 99% sequence identity, or even 100% sequence identity with SEQ ID NO: 1, 13 or 15;
   (ii) a nucleic acid coding for a protein having at least 95%, at least 98%, at least 99% sequence identity, or even 100% sequence identity with SEQ ID NO: 2 or 16; and/or
   (iii) a nucleic acid capable of hybridizing under stringent conditions with any of the nucleic acids according to (i) or (ii) or a complementary sequence thereof;
      operably linked with
(b) a promoter sequence operably linked to the nucleic acid of (a); preferably as depicted in SEQ ID NO: 7, or a functional fragment thereof, or an orthologue or a paralogue thereof; and preferably
(c) a transcription termination sequence is a further embodiment of the invention.

A further preferred embodiment is a recombinant vector construct comprising:
(a) (i) a flavodoxin nucleic acid having at least 60% identity, preferably at least 70% sequence identity, at least 80%, at least 90%, at least 95%, at least 98%, at least 99% sequence identity, or even 100% sequence identity with SEQ ID NO: 1, 13 or 15 or a functional fragment thereof, or an orthologue or a paralogue thereof;
   (ii) a nucleic acid coding for a flavodoxin protein having at least 60% identity, preferably at least 70% sequence identity, at least 80%, at least 90%, at least 95%, at least 98%, at least 99% sequence identity, or even 100% sequence identity with SEQ ID NO: 2 or 16, a functional fragment thereof, an orthologue or a paralogue thereof; and/or
   (iii) a nucleic acid capable of hybridizing under stringent conditions with any of the nucleic acids according to (i) or (ii) or a complementary sequence thereof;
      operably linked with
(b) a transit nucleic acid sequence encoding a transit peptide; preferably as depicted in SEQ ID NO: 3, 8 or 9;
(c) a promoter sequence operably linked to the nucleic acids of (a) and (b); preferably as depicted in SEQ ID NO: 7, or a functional fragment thereof, or an orthologue or a paralogue thereof; and preferably
(d) a transcription termination sequence.

Furthermore, a recombinant vector construct is provided comprising:
(a) (i) a flavodoxin nucleic acid having at least 95%, at least 98%, at least 99% sequence identity, or even 100% sequence identity with SEQ ID NO: 1, 13 or 15;
   (ii) a nucleic acid coding for a protein having at least 95%, at least 98%, at least 99% sequence identity, or even 100% sequence identity with SEQ ID NO: 2 or 16; and/or
   (iii) a nucleic acid capable of hybridizing under stringent conditions with any of the nucleic acids according to (i) or (ii)
      operably linked with
(b) a transit nucleic acid sequence encoding a transit peptide; preferably as depicted in SEQ ID NO: 3, 8 or 9, wherein the transit peptide and the protein encoded by the flavodoxin nucleic acid are in functional linkage with each other;
(c) a promoter sequence operably linked to the nucleic acids of (a) and (b); preferably as depicted in SEQ ID NO: 7;
   and preferably
(d) a transcription termination sequence, where the transcription termination sequence is in functional linkage with the flavodoxin nucleic acid.

A preferred embodiment of the present invention is a vector construct comprising SEQ ID NO: 5, 12, 14 or 17. Preferably the expression vector comprises SEQ ID NO: 5, 12, 14 or 17 and promoter sequence as represented by SEQ ID NO: 7 operably linked to SEQ ID NO: 5, 12, 14 or 17.

The vector constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the vector construct may optionally comprise a selectable marker gene. Examples for selectable marker gene are described herein. The marker genes may be removed or excised from the transgenic cell once they are no longer needed. Techniques for marker removal are known in the art, useful techniques are described herein.

According to another embodiment, the present invention provides a method for enhancing one or more yield-related traits in plants relative to control plants, comprising increasing the expression in a plant of an exogenous nucleic acid encoding a flavodoxin polypeptide as defined herein and optionally selecting for plants having one or more enhanced yield-related traits wherein said nucleic acid is operably linked to a particular promoter as described herein and the flavodoxin polypeptide is expressed specifically by the use of a particular promoter.

A further embodiment of the present invention is a method for enhancing one or more yield-related traits in plants relative to control plants, comprising increasing the expression in a plant of an exogenous nucleic acid encoding a transit peptide and a flavodoxin polypeptide and optionally selecting for plants having one or more enhanced yield-related traits, wherein said nucleic acid is operably linked to a particular promoter as described herein and the flavodoxin polypeptide is expressed specifically by the use of a particular promoter and targeted to the plastid(s). Preferably, the expression of the exogenous nucleic acid is under the control of an endogenous or exogenous promoter sequence.

Preferably said one or more enhanced yield-related traits comprise increased yield relative to control plants, and preferably comprise increased biomass and/or increased seed yield relative to control plants, and preferably comprise increased aboveground biomass, increased below-ground biomass, increased seed yield and/or increased sugar yield (either as harvestable sugar per plant, per fresh weight, per dry weight or per area) relative to control plants.

In a preferred embodiment the seed yield is increased.

In another preferred embodiment the above-ground biomass is increased.

Performance of the methods of the invention results in plants having an increased yield-related trait relative to the yield-related trait of control plants.

The inventive methods for enhancing one or more yield-related traits in plants as described herein comprising introducing, preferably by recombinant methods, and expressing in a plant the nucleic acid(s) and/or constructs as defined herein, and preferably the further step of growing the plants and optionally the step of harvesting the plants or part(s) thereof.

In one embodiment the increased yield-related trait is increased seed yield, preferably increased harvest index, increased seed filling, increased total number of seed, increased total weight of the seed and improved timing, quantity and quality of flowering. More preferably the increased yield-related trait is increased harvest index, increased seed filling and/or increased total weight of the seed.

In another embodiment the increased yield-related trait is increased biomass, in particular aboveground biomass, preferably stem biomass, relative to the aboveground biomass, and in particular stem biomass, of control plants and/or increased root biomass relative to the root biomass of control plants and/or increased beet biomass relative to the beet biomass of control plants. Moreover, it is particularly contemplated that the sugar content (in particular the sucrose content) in the aboveground parts, particularly stem (in particular of sugarcane plants) and/or in the belowground parts, in particular in roots including taproots and tubers, and/or in beets (in particular in sugar beets) is increased relative to the sugar content (in particular the sucrose content) in corresponding part(s) of the control plant.

Preferred aboveground biomass is stem biomass. Enhanced stem biomass can be displayed in an increase in stem length, stem width or breadth, stem density, stem weight, stem diameter, number of nodes and/or internodes, diameter or amount or density of stem vasculature or vascular bundles, in particular phloem and or xylem. Moreover, the sap content of the stem is preferably enhanced. Furthermore, the sucrose content, preferably the stem sucrose content is preferably enhanced.

In particular, the methods of the present invention may be performed under stress or non-stress conditions. Stress conditions are preferably abiotic stress conditions, more preferably drought, salinity and/or cold or hot temperatures and/or nutrient use due to one or more nutrient deficiency such as nitrogen deficiency, most preferably drought and/or nitrogen deficiency.

In a preferred embodiment the methods of the invention are performed using plants in need of increased abiotic stress-tolerance for example tolerance to drought, salinity and/or cold or hot temperatures and/or nutrient use due to one or more nutrient deficiency such as nitrogen deficiency.

In an example, the methods of the present invention may be performed under stress conditions, such as drought or mild drought, to give plants having increased yield relative to control plants. Preferably, when subjected to drought stress the transgenic plants having increased biomass, preferably aboveground biomass, and/or increased seed yield relative to control plants.

In another example, the methods of the present invention may be performed under stress conditions such as nutrient deficiency to give plants having increased yield relative to control plants. Nutrient deficiency may result from a lack of nutrients such as nitrogen, phosphates and other phosphorous-containing compounds, potassium, calcium, magnesium, manganese, iron and boron, amongst others. Preferably, when subjected to nutrient deficiency the transgenic plants having increased biomass, preferably aboveground biomass, and/or increased seed yield relative to control plants.

In yet another example, the methods of the present invention may be performed under stress conditions such as salt stress to give plants having increased yield relative to control plants. The term salt stress is not restricted to common salt (NaCl), but may be any one or more of: NaCl, KCl, LiCl, MgCl₂, CaCl₂, amongst others. Preferably, when subjected to salt stress the transgenic plants having increased biomass, preferably aboveground biomass, and/or increased seed yield relative to control plants.

In yet another example, the methods of the present invention may be performed under stress conditions such as cold stress or freezing stress to give plants having increased yield relative to control plants. Preferably, when subjected to cold stress the transgenic plants having increased biomass, preferably aboveground biomass, and/or increased seed yield relative to control plants.

In another preferred embodiment the methods of the present invention are performed under non-stress conditions.

In yet another embodiment, there is provided a method for enhancing one or more yield-related traits in plants, comprising introducing and expressing in a plant one or more of any of the exogenous nucleic acids given in Table 2 and/or the sequence listing, or comprising introducing and expressing in a plant a functional fragment, an orthologue, paralogue or homologue of any of the nucleic acid sequences given in Table 2 and/or the sequence listing or
(i) an exogenous nucleic acid having at least 60% identity with SEQ ID NO: 1, 13 or 15, or a functional fragment thereof, an orthologue or a paralogue thereof; or
(ii) an exogenous nucleic acid encoding a protein having at least 60% identity with SEQ ID NO: 2 or 16, or a functional fragment thereof, an orthologue or a paralogue thereof; or
(iii) an exogenous nucleic acid capable of hybridizing under stringent conditions with any of the nucleic acids according to (i) or (ii) or a complementary sequence thereof; or
(iv) an exogenous nucleic acid encoding a polypeptide with the biological activity of a flavodoxin or a ferredoxin; or
(v) an exogenous nucleic acid encoding the same polypeptide as the nucleic acids of (i) to (iv) above, but differing from the nucleic acids of (i) to (iv) above due to the degeneracy of the genetic code; or
(vi) an exogenous nucleic acid combining the features of the nucleic acids of any two of (i) to (iv) above.

Preferably, the exogenous nucleic acid also encodes for any of the transit peptides given in Table 3.

A preferred method for increasing expression of an exogenous nucleic acid encoding a flavodoxin polypeptide is by introducing and expressing in a plant a nucleic acid encoding a flavodoxin polypeptide, even more preferably wherein said nucleic acid is operably linked to a particular promoter as described herein and the flavodoxin polypeptide is targeted to the plastids.

According to one embodiment, there is provided a method for improving yield-related traits as provided herein in plants relative to control plants, comprising increasing the expression in a plant of an exogenous nucleic acid encoding a flavodoxin polypeptide as defined herein, wherein said nucleic acid is operably linked to a particular promoter as described herein and the flavodoxin polypeptide is targeted to the plastids.

In another embodiment, there is provided a method for enhancing one or more yield-related traits in plants, comprising introducing and expressing in a plant a functional fragment, orthologue, paralogue, or splice variant of any of the nucleic acids given in Table 2 and/or the sequence listing

In yet another embodiment, there is provided a method for enhancing one or more yield-related traits in plants, comprising introducing and expressing in a plant an allelic variant of one or more of any of the nucleic acids given in Table 2 and/or the sequence listing,

Hence, a preferred embodiment is a method for enhancing one or more yield-related traits in a plant relative to control plants, comprising increasing the expression in a plant of an exogenous nucleic acid encoding a transit peptide and a flavodoxin polypeptide, wherein the expression is under the control of a promoter sequence operably linked to the nucleic acid encoding the transit peptide and the flavodoxin polypeptide. Preferably, the promoter sequence comprises the nucleotide sequence of the protochlorophyllide reductase promoter promoter, or functional fragments or derivatives thereof. The protochlorophyllide reductase promoter promoter preferably comprises the sequence of SEQ ID NO: 7.

In a preferred embodiment, the transit peptide targets the flavodoxin polypeptide to a plastid, preferably to a chloroplast. Preferably, the chloroplast transit peptide is selected from the transit peptides listed in Table 3.

Preferably, the flavodoxin polypeptide is encoded by a nucleic acid sequence selected from the group of nucleic acid sequences listed in Table 2 and/or the sequence listing. More preferably, the flavodoxin polypeptide is from Anabaena sp., preferably Anabaena PCC7119, or Synechocystis sp., preferably Synechocystis sp. PCC 6803. Most preferred, the transit peptide is encoded by
(i) an exogenous nucleic acid having at least 60% identity with SEQ ID NO: 1, 13 or 15 or a functional fragment thereof, an orthologue or a paralogue thereof;
(ii) an exogenous nucleic acid encoding a protein having at least 60% identity with SEQ ID NO: 2 or 16, or a functional fragment thereof, an orthologue or a paralogue thereof; and/or by
(iii) an exogenous nucleic acid capable of hybridizing under stringent conditions with any of the nucleic acids according to (i) or (ii) or a complementary sequence thereof.

Most preferred is the flavodoxin polypeptide being encoded by
(i) an exogenous nucleic acid having at least 60% identity with SEQ ID NO: 1, 13 or 15 or a functional fragment thereof, an orthologue or a paralogue thereof;
(ii) an exogenous nucleic acid encoding a protein having at least 60% identity with SEQ ID NO: 2 or 16, or a functional fragment thereof, an orthologue or a paralogue thereof; and/or by
(iii) an exogenous nucleic acid capable of hybridizing under stringent conditions with any of the nucleic acids according to (i) or (ii) or a complementary sequence thereof.

A method for enhancing one or more yield-related traits in a plant relative to control plants, preferably comprises
(a) stably transforming a plant cell with an expression cassette comprising an exogenous nucleic acid encoding a transit peptide and encoding a flavodoxin polypeptide, wherein the flavodoxin polypeptide is encoded by
   (i) an exogenous nucleic acid having at least 60% identity with SEQ ID NO: 1, 13 or 15 or a functional fragment thereof, an orthologue or a paralogue thereof;
   (ii) an exogenous nucleic acid coding for a protein having at least 60% identity with SEQ ID NO: 2 or 16, or a functional fragment thereof, an orthologue or a paralogue thereof; and/or
   (iii) an exogenous nucleic acid capable of hybridizing under stringent conditions with any of the nucleic acids according to (i) or (ii) or a complementary sequence thereof; in functional linkage with a promoter sequence;
(b) regenerating the plant from the plant cell; and
(c) expressing said exogenous nucleic acid.

Preferably, the transit peptide is selected from the transit peptides shown in Table 3, more preferably it is encoded by the nucleic acids of SEQ ID NO: 3, 8 or 9 or has the sequence as disclosed in SEQ ID NO: 4 or 10. Preferably, the promoter sequence comprises a nucleic acid sequence as represented by SEQ ID NO: 7.

As an alternative to the nucleic acid of SEQ ID NO: 9 the nucleic acid of SEQ ID NO: 8 encoding the transit peptide of the variant of SEQ ID NO: 10 can be used.

Preferably, the plant used in the method of the present invention is a dicotyledonous or monocotyledonous plant. Preferably, the plant is a Poaceae. More preferably, the monocotyledonous plant is of the genus saccharum, preferably selected from the group consisting of Saccharum arundinaceum, Saccharum bengalense, Saccharum edule, Saccharum munja, Saccharum officinarum, Saccharum procerum, Saccharum ravennae, Saccharum robustum, Saccharum sinense, and Saccharum spontaneum.

Performance of the methods of the invention gives plants having one or more enhanced yield-related traits. In particular performance of the methods of the invention gives plants having increased early vigour and/or increased yield, especially increased biomass and/or increased seed yield relative to control plants. The terms “early vigour” “yield”, “biomass”, and “seed yield” are described in more detail in the “definitions” section herein.

The present invention thus provides a method for increasing yield-related traits, especially biomass and/or seed yield of plants, relative to control plants, which method comprises increasing the expression in a plant of an exogenous nucleic acid as described herein. Preferably, the exogenous nucleic acid also encodes a transit peptide, preferably, a chloroplast transit sequence. Preferably, said enhanced yield-related trait comprises enhanced biomass and/or increased seed yield relative to control plants, and preferably comprise enhanced aboveground biomass and/or increased seed yield relative to control plants.

According to a preferred embodiment of the present invention, performance of the methods of the invention gives plants having an increased growth rate relative to control plants. Therefore, according to the present invention, there is provided a method for increasing the growth rate of plants, which method comprises increasing expression in a plant of a nucleic acid encoding a flavodoxin polypeptide as defined herein.

Performance of the methods of the invention gives plants grown under non-stress conditions and/or under stress conditions increased yield-related traits relative to control plants grown under comparable conditions. Therefore, according to the present invention, there is provided a method for increasing one or more yield-related traits in plants grown under non-stress conditions and/or under stress conditions, which method comprises increasing expression in a plant of a nucleic acid encoding a flavodoxin polypeptide. Preferably, the method comprises the step of introducing an exogenous nucleic acid encoding a flavodoxin polypeptide, and preferably a transit peptide, in said plant, preferably under the control of an endogenous or exogenous promoter sequence as described herein. Preferably, said enhanced yield-related trait is obtained under conditions of drought stress, salt stress or nitrogen deficiency

Performance of the methods of the invention gives plants grown under conditions of drought, increased yield-related traits relative to control plants grown under comparable conditions. Therefore, according to the present invention, there is provided a method for increasing yield-related traits in plants grown under conditions of drought which method comprises increasing expression in a plant of an exogenous nucleic acid encoding a flavodoxin polypeptide, wherein said nucleic acid is operably linked to a particular promoter as described herein and the flavodoxin polypeptide is targeted to the plastids.

Performance of the methods of the invention gives plants grown under conditions of nutrient deficiency, particularly under conditions of nitrogen deficiency, increased yield-related traits relative to control plants grown under comparable conditions. Therefore, according to the present invention, there is provided a method for increasing yield-related traits in plants grown under conditions of nutrient deficiency, which method comprises increasing expression in a plant of an exogenous nucleic acid encoding a flavodoxin polypeptide, wherein said nucleic acid is operably linked to a particular promoter as described herein and the flavodoxin polypeptide is targeted to the plastids.

Performance of the methods of the invention gives plants grown under conditions of salt stress, increased yield-related traits relative to control plants grown under comparable conditions. Therefore, according to the present invention, there is provided a method for increasing yield-related traits in plants grown under conditions of salt stress, which method comprises increasing expression in a plant of an exogenous nucleic acid encoding a flavodoxin polypeptide, wherein said nucleic acid is operably linked to a particular promoter as described herein and the flavodoxin polypeptide is targeted to the plastids.

In one embodiment of the invention, seed yield is increased.

In another embodiment of the invention, above ground biomass is increased, preferably stem, stalk and/or sett biomass, more preferably in Poaceae, even more preferably in a Saccharum species, most preferably in sugarcane, and optionally below-ground biomass and/or root growth is not increased compared to control plants.

In a further embodiment the total harvestable sugar, preferably glucose, fructose and/or sucrose, is increased, preferably in addition to increased other yield-related traits as defined herein, for example biomass, and more preferably also in addition to an increase in sugar content, preferably glucose, fructose and/or sucrose content.

Methods for increasing expression of nucleic acids or genes, or gene products, are well documented in the art and examples are provided herein.

As mentioned above, a preferred method for modulating expression of a nucleic acid encoding a flavodoxin polypeptide is by introducing and expressing in a plant a nucleic acid encoding a flavodoxin polypeptide; however the effects of performing the method, i.e. enhancing yield-related traits may also be achieved using other well-known techniques, including but not limited to T-DNA activation tagging, TILLING, homologous recombination. A description of these techniques is provided in the definitions section.

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

A preferred embodiment of the present invention is the use of an expression construct according or a recombinant expression vector described herein in a method for making a transgenic plant having an enhanced yield-related trait, preferably increased biomass and/or increased seed yield, relative to control plants, and more preferably increased above-ground biomass and/or increased seed yield relative to control plants.

Thus, a preferred embodiment is a transgenic plant, transgenic plant part, or transgenic plant cell obtainable by a method for enhancing one or more yield-related traits in a plant relative to control plants or by a method for the production of transgenic plants, as described herein, wherein said transgenic plant, transgenic plant part, or transgenic plant cell expresses an exogenous nucleic acid encoding a transit peptide and a flavodoxin polypeptide under the control of a promoter sequence as described herein.

Preferably, the transgenic plant, transgenic plant part, or transgenic plant cell is transformed with an expression construct or with a recombinant expression vector described herein.

In a preferred embodiment the plant, plant part, seed, sett or propagule of the invention has one or more increased yield-related trait(s) under non-stress conditions and/or under conditions of drought and 7 or nitrogen deficiency, more preferably under non-stress conditions.

Most preferred, the transgenic plant, transgenic plant part or transgenic plant cell has an enhanced yield-related trait, preferably an enhanced biomass and/or increased seed yield relative to control plants.

The invention also includes host cells containing an exogenous isolated nucleic acid encoding a flavodoxin polypeptide as defined above. In one embodiment host cells according to the invention are plant cells, yeasts, bacteria or fungi. Preferred bacterial host cells are Escherichia coli or Agrobacterium. Host plants for the nucleic acids, construct, expression cassette or the vector used in the method according to the invention are, in principle, advantageously all plants which are capable of synthesizing the polypeptides used in the inventive method. In a particular embodiment the plant cells of the invention overexpress the nucleic acid molecule of the invention.

Thus, one embodiment of the present invention is an exogenous nucleic acid encoding a transit peptide and a flavodoxin polypeptide, as described herein, operatively linked to a promoter sequence, preferably a protochlorophyllide reductase promoter promoter, as described herein, comprised in a host cell, wherein the host cell is selected from the group consisting of plant cell, bacterial cell, yeast cell, fungal cell, and mammalian cell, preferably, plant cell, more preferably a Poaceae cell, even more preferably a cell of the genus Saccharum, most preferably a sugarcane cell.

The methods of the invention are advantageously applicable to any plant, in particular to any plant as defined herein. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to an embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, potato and tobacco. According to another embodiment of the present invention, the plant is a monocotyledonous plant. Examples of monocotyledonous plants include sugarcane. According to another embodiment of the present invention, the plant is a cereal. Examples of cereals include rice, maize, wheat, barley, millet, rye, triticale, sorghum, emmer, spelt, einkorn, teff, milo and oats. In a particular embodiment the plants of the invention or used in the methods of the invention are selected from the group consisting of maize, wheat, rice, soybean, cotton, oilseed rape including canola, sugarcane, sugar beet and alfalfa.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Preferred plants are Poaceae. Most preferred plant is sugarcane, preferably of the genus saccharum. More preferred is a plant selected from the group consisting of Saccharum arundinaceum, Saccharum bengalense, Saccharum edule, Saccharum munja, Saccharum officinarum, Saccharum procerum, Saccharum ravennae, Saccharum robustum, Saccharum sinense, and Saccharum spontaneum.

With respect to the sequences of the invention or useful in the methods, constructs, plants, harvestable parts and products of the invention, in one embodiment a nucleic acid or a polypeptide sequence originating not from higher plants is used in the methods of the invention or the expression construct useful in the methods of the invention. In another embodiment a nucleic acid or a polypeptide sequence of plant origin is used in the methods, constructs, plants, harvestable parts and products of the invention because said nucleic acid and polypeptides has the characteristic of a codon usage optimised for expression in plants, and of the use of amino acids and regulatory sites common in plants, respectively. The plant of origin may be any plant, but preferably those plants as described herein. In yet another embodiment a nucleic acid sequence originating not from higher plants but artificially altered to have the codon usage of higher plants is used in the expression construct useful in the methods of the invention.

According to another embodiment, the present invention provides a method for producing plants having one or more enhanced yield-related traits relative to control plants, wherein said method comprises the steps of increasing the expression in said plant of a nucleic acid encoding a flavodoxin polypeptide as described herein and optionally selecting for plants having one or more enhanced yield-related traits.

According to another embodiment, the present invention provides a method for producing plants having one or more enhanced yield-related traits relative to control plants, wherein said method comprises the steps of increasing the expression in said plant of a nucleic acid encoding transit peptide and a flavodoxin polypeptide as described herein, wherein said nucleic acid is operably linked to a particular promoter as described herein, and optionally selecting for plants having one or more enhanced yield-related traits.

Thus the invention furthermore provides plants or host cells transformed with a construct as described herein. In particular, the invention provides plants transformed with a construct as described herein, which plants have increased yield-related traits as described herein.

A preferred embodiment is therefore a method for the production of a transgenic plant, transgenic plant part, or transgenic plant cell having an enhanced yield-related traits relative to control plants, preferably increased biomass and/or seed yield, comprising:
(a) introducing a recombinant vector construct described herein into a plant, a plant part, or a plant cell;
(b) generating a transgenic plant, transgenic plant part, or transgenic plant cell from the transformed plant, transformed plant part or transformed plant cell; and
(c) expressing the exogenous nucleic acid encoding the transit peptide and the flavodoxin polypeptide.

In one embodiment the methods for the production of a transgenic plant, transgenic plant part, or transgenic plant cell having an enhanced yield-related traits relative to control plants, comprises the step of harvesting the seeds of the transgenic plant and planting the seeds and growing the seeds to plants, wherein the seeds comprises the exogenous nucleic acid encoding the transit peptide and the flavodoxin polypeptide, and the promoter sequence operably linked thereto.

In another embodiment the methods of the invention are methods for the production of a transgenic Poaceae plant, preferably a Saccharum species plant, a transgenic part thereof, or a transgenic plant cell thereof, having one or more enhanced yield-related traits relative to control plants, comprises the step of harvesting setts from the transgenic plant and planting the setts and growing the setts to plants, wherein the setts comprises the exogenous nucleic acid encoding the POI polypeptide and the promoter sequence operably linked thereto.

The invention also provides a method for the production of transgenic plants having enhanced biomass, preferably aboveground biomass, and/or increased seed yield relative to control plants, comprising introduction and expression in a plant of any nucleic acid encoding a flavodoxin polypeptide as defined herein wherein said nucleic acid is operably linked to a particular promoter as described herein and the flavodoxin polypeptide is targeted to the plastids.

More specifically, the present invention provides a method for the production of transgenic plants having one or more enhanced yield-related traits, particularly increased biomass and/or seed yield, which method comprises:
(i) introducing and expressing in a plant or plant cell a flavodoxin polypeptide-encoding nucleic acid or a genetic construct comprising a flavodoxin polypeptide-encoding nucleic acid; and
(ii) cultivating the plant cell under conditions promoting plant growth and development, preferably promoting plant growth and development of plants having one or more enhanced yield-related traits relative to control plants.

The nucleic acid of (i) may be any of the nucleic acids capable of encoding a flavodoxin polypeptide as described herein. Preferably the nucleic acid also encodes a transit peptide targeting the flavodoxin to the plastid and preferably, the nucleic acid is operably linked to a promoter sequence described herein.

Cultivating the plant cell under conditions promoting plant growth and development, may or may not include regeneration and/or growth to maturity. Accordingly, in a particular embodiment of the invention, the plant cell transformed by the method according to the invention is regenerable into a transformed plant. In another particular embodiment, the plant cell transformed by the method according to the invention is not regenerable into a transformed plant, i.e. cells that are not capable to regenerate into a plant using cell culture techniques known in the art. While plants cells generally have the characteristic of totipotency, some plant cells cannot be used to regenerate or propagate intact plants from said cells. In one embodiment of the invention the plant cells of the invention are such cells. In another embodiment the plant cells of the invention are plant cells that do not sustain themselves in an autotrophic way. One example are plant cells that do not sustain themselves through photosynthesis by synthesizing carbohydrate and protein from such inorganic substances as water, carbon dioxide and mineral salt.

The nucleic acid may be introduced directly into a plant cell or into the plant itself (including introduction into a tissue, organ or any other part of a plant). According to a preferred feature of the present invention, the nucleic acid is preferably introduced into a plant or plant cell by transformation. The term “transformation” is described in more detail in the “definitions” section herein.

In a preferred embodiment the methods of the invention are performed using plants in need of increased abiotic stress-tolerance for example tolerance to drought, salinity and/or cold or hot temperatures and/or nutrient use due to one or more nutrient deficiency such as nitrogen deficiency.

In one embodiment the present invention extends to any plant cell or plant produced by any of the methods described herein, and to all plant parts and propagules thereof.

The present invention encompasses plants or parts thereof (including seeds and/or setts) obtainable by the methods according to the present invention. The plants or plant parts or plant cells comprise a nucleic acid transgene encoding a flavodoxin polypeptide as defined above, preferably in a genetic construct such as an expression cassette. The present invention extends further to encompass the progeny of a primary transformed or transfected cell, tissue, organ or whole plant that has been produced by any of the aforementioned methods, the only requirement being that progeny exhibit substantially the same genotypic and/or phenotypic characteristic(s) as those produced by the parent in the methods according to the invention.

In a further embodiment the invention extends to seeds and/or setts exogenously comprising the expression cassettes of the invention, the genetic constructs of the invention, or the nucleic acids encoding
the flavodoxin polypeptide
and/or the flavodoxin functional fragment,
derivative,
orthologue, and/or
paralogue thereof,
   as described herein and operably linked to a particular promoter as described herein. Typically a plant grown from the seed or sett of the invention will also show enhanced yield-related traits.

The invention also extends to harvestable parts of a transgenic plant of the present invention, such as, but not limited to seeds, leaves, fruits, flowers, stems, setts, roots, rhizomes, tubers and bulbs, wherein the harvestable parts comprise the construct of the invention, and/or an exogenous nucleic acid encoding a flavodoxin polypeptide operably linked to a particular promoter as described herein and/or the flavodoxin polypeptide as defined herein with targeting to the plastid and expressed specifically by the use of a particular promoter. and/or

In particular, such harvestable parts are roots such as taproots, rhizomes, fruits, stems, setts, beets, tubers, bulbs, leaves, flowers and/or seeds. Preferred harvestable parts are seed and/or stem cuttings (like setts of sugarcane but not limited to setts).

In another embodiment aboveground parts or aboveground harvestable parts or aboveground biomass are to be understood as aboveground vegetative biomass not including seeds and/or fruits.

In a further embodiment the invention relates to a transgenic pollen grain comprising the construct of the invention and/or a haploid derivate of the plant cell of the invention. Although in one particular embodiment the pollen grain of the invention can not be used to regenerate an intact plant without adding further genetic material and/or is not capable of photosynthesis, said pollen grain of the invention may have uses in introducing the enhanced yield-related trait into another plant by fertilizing an egg cell of the other plant using a live pollen grain of the invention, producing a seed from the fertilized egg cell and growing a plant from the resulting seed. Further pollen grains find use as marker of geographical and/or temporal origin.

The invention furthermore relates to products derived or produced from a transgenic plant described herein or one or more harvestable product of a transgenic plant described herein, preferably directly derived or directly produced, from one or more harvestable part(s) of such a transgenic plant. Preferred products are dry pellets, pressed stems, setts, meal or powders, fibres, cloth, paper or cardboard containing fibres produced by the plants of the invention, oil, fat and fatty acids, starch, carbohydrates,—including starches, paper or cardboard containing carbohydrates produced by the plants of the invention—, sap, juice, chaff, or proteins. Preferred carbohydrates are starches, cellulose and/or sugars, preferably sucrose. Also preferred products are residual dry fibers, e.g., of the stem (like bagasse from sugarcane after cane juice removal), molasse, or filtercake, preferably from sugarcane. Said products can be agricultural products.

Preferably, the product comprises—for example as an indicator of the particular quality of the product—the construct of the invention, an exogenous nucleic acid encoding a flavodoxin polypeptide as described herein and/or an exogenous flavodoxin polypeptide as described herein, wherein said nucleic acid is operably linked to a particular promoter as described herein and the flavodoxin polypeptide is targeted to the plastids and expressed specifically by the use of a particular promoter.

In another embodiment the invention relates to anti-counterfeit milled seed and/or milled stem having as an indication of origin and/or as an indication of producer a plant cell of the invention and/or the construct of the invention, wherein milled stem preferably is milled Poaceae stem, more preferably milled sugarcane.

The invention also includes methods for manufacturing a product comprising a) growing the plants of the invention and b) producing said product from or by the plants of the invention or parts thereof, including stem and/or seeds. In a further embodiment the methods comprise the steps of a) growing the plants of the invention, b) removing the harvestable parts as described herein from the plants and c) producing said product from, or with the harvestable parts of plants according to the invention. Preferably, the product comprises the genetic construct, nucleic acid and/or polypeptide of the invention as described herein. More preferably the product is produced from seeds or the stem of the transgenic plant, preferably from the seeds.

In one embodiment, the method for manufacturing a product comprising a) growing the Poaceae plants of the invention, preferably the plant being a Saccharum species and more preferably sugarcane, b) obtaining the stem from the plants of the invention, and c) cutting the stem into pieces, preferably into pieces suitable as propagation material, preferably into one or more setts. Preferably, the setts comprise the construct, nucleic acid and/or polypeptide of the invention as described herein.

In another embodiment, the method for manufacturing a product comprising a) growing the Poaceae pplants of the invention, preferably the plant being a Saccharum species and more preferably sugarcane, b) obtaining the stem from the plants of the invention or parts thereof, and c) extracting the juice, preferably the cane juice from the stem and/or extracting the residual fibers after juice extraction, and optionally d) extracting sugar, preferably, sucrose, from the juice of the stem.

In a preferred embodiment the methods of the invention are performed using plants in need of increased abiotic stress-tolerance for example tolerance to drought, salinity and/or cold or hot temperatures and/or nutrient use due to one or more nutrient deficiency such as nitrogen deficiency.

In one embodiment the method of the invention is a method for manufacturing cloth by a) growing the plants of the invention that are capable of producing fibres usable in cloth making, e.g. cotton, b) removing the harvestable parts as described herein from the plants, and c) producing fibres from said harvestable part and d) producing cloth from the fibres of c). Another embodiment of the invention relates to a method for producing feedstuff for bioreactors, fermentation processes or biogas plants, comprising a) growing the plants of the invention, b) removing the harvestable parts as described herein from the plants and c) producing feedstuff for bioreactors, fermentation processes or biogas plants. In a preferred embodiment the method of the invention is a method for producing alcohol(s) from plant material comprising a) growing the plants of the invention, b) removing the harvestable parts as described herein from the plants and c) optionally producing feedstuff for fermentation process, and d)—following step b) or c)—producing one or more alcohol(s) from said feedstuff or harvestable parts, preferably by using microorganisms such as fungi, algae, bacteria or yeasts, or cell cultures. A typical example would be the production of ethanol using carbohydrate containing harvestable parts, for example corn seed, sugarcane stem parts or beet parts of sugar beet, or products derived therefrom for example juice or sap from sugarcane or sugar beet or corn starch or corn starch syrup. In one embodiment, the product is produced from the stem of the transgenic plant. In another embodiment the product is produced from the seed of the plant.

In another embodiment the method of the invention is a method for the production of one or more polymers comprising a) growing the plants of the invention, b) removing the harvestable parts as described herein from the plants and c) producing one or more monomers from the harvestable parts, optionally involving intermediate products, d) producing one or more polymer(s) by reacting at least one of said monomers with other monomers or reacting said monomer(s) with each other. In another embodiment the method of the invention is a method for the production of a pharmaceutical compound comprising a) growing the plants of the invention, b) removing the harvestable parts as described herein from the plants and c) producing one or more monomers from the harvestable parts, optionally involving intermediate products, d) producing a pharmaceutical compound from the harvestable parts and/or intermediate products. In another embodiment the method of the invention is a method for the production of one or more chemicals comprising a) growing the plants of the invention, b) removing the harvestable parts as described herein from the plants and c) producing one or more chemical building blocks such as but not limited to acetate, pyruvate, lactate, fatty acids, sugars, amino acids, nucleotides, carotenoids, terpenoids or steroids from the harvestable parts, optionally involving intermediate products, d) producing one or more chemical(s) by reacting at least one of said building blocks with other building block or reacting said building block(s) with each other.

The present invention is also directed to a product obtained by a method for manufacturing a product, as described herein.

In one embodiment the products produced by the methods of the invention are plant products such as, but not limited to, a foodstuff, feedstuff, a food supplement, feed supplement, fiber, cosmetic or pharmaceutical. In another embodiment the methods for production are used to make agricultural products such as, but not limited to fibres, plant extracts, meal or presscake and other leftover material after one or more extraction processes, flour, proteins, amino acids, carbohydrates, fats, oils, polymers, vitamins, and the like. Preferred carbohydrates are sugars, preferably sucrose. In one embodiment the agricultural product is selected from the group consisting of 1) fibres, 2) timber, 3) plant extracts, 4) meal or presscake or other leftover material after one or more extraction processes, 5) flour, 6) proteins, 7) carbohydrates, 8) fats, 9) oils, 10) polymers e.g. cellulose, starch, lignin, lignocellulose, and 11) combinations and/or mixtures of any of 1) to 10). In a preferable embodiment the product or agricultural product does generally not comprise living plant cells, does comprise the expression cassette, genetic construct, protein and/or polynucleotide as described herein.

Preferably, the product comprises the genetic construct, nucleic acid and/or polypeptide of the invention as described herein.

In yet another embodiment the polynucleotides and/or the polypeptides and/or the genetic constructs of the invention are comprised in an agricultural product. In a particular embodiment the nucleic acid sequences and/or protein sequences and/or the genetic constructs of the invention may be used as product markers, for example where an agricultural product was produced by the methods of the invention. Such a marker can be used to identify a product to have been produced by an advantageous process resulting not only in a greater efficiency of the process but also improved quality of the product due to increased quality of the plant material and harvestable parts used in the process. Such markers can be detected by a variety of methods known in the art, for example but not limited to PCR based methods for nucleic acid detection or antibody based methods for protein detection.

The present invention also encompasses use of constructs comprising nucleic acids encoding flavodoxin polypeptides and operably linked a particular promoter as described herein and use of these flavodoxin polypeptides expressed specifically by the use of a particular promoter in enhancing any of the aforementioned yield-related traits in plants. For example, constructs comprising nucleic acids encoding flavodoxin polypeptide and operably linked a particular promoter as described herein, or the flavodoxin polypeptides themselves expressed specifically by the use of a particular promoter, may find use in breeding programmes in which a DNA marker is identified which may be genetically linked to a flavodoxin polypeptide-encoding gene—promoter combination as described herein. The nucleic acids/gene—promoter combination of the invention, or the flavodoxin polypeptides themselves expressed specifically by the use of a particular promoter may be used to define a molecular marker. This DNA or protein marker may then be used in breeding programmes to select plants having one or more enhanced yield-related traits as defined herein in the methods of the invention. Furthermore, allelic variants of a flavodoxin polypeptide-encoding nucleic acid/gene operably linked a particular promoter as described herein may find use in markerassisted breeding programmes. The inventive combinations of a particular promoter and nucleic acids encoding flavodoxin polypeptides may also be used as probes for genetically and physically mapping the genomic location of genes that they are a part of, and as markers for traits linked to those genes and their insertion sites. Such information may be useful in plant breeding in order to develop lines with desired phenotypes.

A preferred embodiment is a method for breeding a plant with one or more enhanced yield-related traits comprising
(a) crossing a transgenic plant of the invention or a transgenic plant obtainable by any of the methods described herein with a second plant;
(b) obtaining seed from the cross of step (a);
(c) planting said seeds and growing the seeds to plants; and
(d) selecting from said plants, plants exogenously expressing the nucleic acid encoding flavodoxin polypeptide described herein, preferably encoding the transit peptide and the flavodoxin polypeptide, wherein the nucleic acid is preferably functionally linked to a promoter sequence described herein.

Optionally, the method for breeding further comprises the step of (e) producing propagation material from the plants expressing the nucleic acid encoding the transit peptide and the flavodoxin polypeptide, wherein the propagation material comprises the genetic construct and/or vector construct of the invention. Preferably, the propagation material being cuttings of the stem or seeds.

Another preferred embodiment is a method for plant improvement comprising
(a) obtaining a transgenic plant by any of the methods of the present invention;
(b) combining within one plant cell the genetic material of at least one plant cell of the plant of a) with the genetic material of at least one cell differing in one or more gene from the plant cells of the plants of a) or crossing the transgenic plant of a) with a second plant;
(c) obtaining seed from at least on plant generated from the one plant cell of b) or the plant of the cross of step (b);
(d) planting said seeds and growing the seeds to plants; and
(e) selecting from said plants, plants expressing under the control of a particular promoter as described herein the nucleic acid encoding the transit peptide and the flavodoxin polypeptide; and optionally
(f) producing propagation material from the plants expressing the nucleic acid encoding the transit peptide and the flavodoxin polypeptide, wherein the propagation material comprises the genetic construct and/or vector construct of the invention.

Preferably, the propagation material being cuttings of the stem or seeds.

In a preferred embodiment the methods of the invention are performed using plants in need of increased abiotic stress-tolerance for example tolerance to drought, salinity and/or cold or hot temperatures and/or nutrient use due to one or more nutrient deficiency such as nitrogen deficiency.

In one embodiment, the total storage carbohydrate content of the plants of the invention, or parts thereof and in particular of the harvestable parts of the plant(s) is increased compared to control plant(s) and the corresponding plant parts of the control plants. Storage carbohydrates are preferably sugars such as but not limited to sucrose, fructose and glucose, and polysaccharides such as but not limited to starches, glucans and fructans.

The total storage carbohydrate content and the content of individual groups or species of carbohydrates may be measured in a number of ways known in the art. For example, the international application published as WO2006066969 discloses in paragraphs [79] to [117] a method to determine the total storage carbohydrate content of sugarcane, including fructan content.

Another method for sugarcane is as follows: The transgenic sugarcane plants are grown for 10 to 15 months, either in the greenhouse or the field. Standard conditions for growth of the plants are used. Stalks of sugarcane plants which are 10 to 15 months old and have more than 10 internodes are harvested. After all of the leaves have been removed, the internodes of the stalk are numbered from top (=1) to bottom (for example=36). A stalk disc approximately 1-2 g in weight is excised from the middle of each internode. The stalk discs of 3 internodes are then combined to give one sample and frozen in liquid nitrogen.

For the sugar extraction, the stalk discs are first comminuted in a Waring blender (from Waring, New Hartford, Conn., USA). The sugars are extracted by shaking for one hour at 95° C. in 10 mM sodium phosphate buffer pH 7.0. Thereafter, the solids are removed by filtration through a 30 μm sieve. The resulting solution is subsequently employed for the sugar determination (see herein below).

The transgenic sugarcane plants are grown for 10 to 15 months. In each case a sugarcane stalk of the transgenic line and a wild-type sugarcane plant is defoliated, the stalk is divided into segments of 3 internodes, and these internode segments are frozen in liquid nitrogen in a sealed 50 ml plastic container. The fresh weight of the samples is determined. The extraction for the purposes of the sugar determination is done as described below.

The glucose, fructose and sucrose contents in the extract obtained in accordance with the sugar extraction method described above is determined photometrically in an enzyme assay via the conversion of NAD+ (nicotinamide adenine dinucleotide) into NADH (reduced nicotinamide adenine dinucleotide). During the reduction, the aromatic character at the nicotinamide ring is lost, and the absorption spectrum thus changes. This change in the absorption spectrum can be detected photometrically. The glucose and fructose present in the extract is converted into glucose-6-phosphate and fructose-6-phosphate by means of the enzyme hexokinase and adenosin triphosphate (ATP). The glucose-6-phosphate is subsequently oxidized by the enzyme glucose-6-phosphate dehydrogenase to give 6-phosphogluconate. In this reaction, NAD+ is reduced to give NADH, and the amount of NADH formed is determined photometrically. The ratio between the NADH formed and the glucose present in the extract is 1:1, so that the glucose content can be calculated from the NADH content using the molar absorption coefficient of NADH (6.3 1 per mmol and per cm lightpath). Following the complete oxidation of glucose-6-phosphate, fructose-6-phosphate, which has likewise formed in the solution, is converted by the enzyme phosphoglucoisomerase to give glucose-6-phosphate which, in turn, is oxidized to give 6-phosphogluconate. Again, the ratio between fructose and the amount of NADH formed is 1:1. Thereafter, the sucrose present in the extract is cleaved by the enzyme sucrase (Megazyme) to give glucose and fructose. The glucose and fructose molecules liberated are then converted with the abovementioned enzymes in the NAD+-dependent reaction to give 6-phosphogluconate. The conversion of one sucrose molecule into 6-phosphogluconate results in two NADH molecules. The amount of NADH formed is likewise determined photometrically and used for calculating the sucrose content, using the molar absorption coefficient of NADH.

The sugarcane stalks are divided into segments of in each case three internodes, as specified above. The internodes are numbered from top to bottom (top=internode 1, bottom=internode 21).

Furthermore transgenic sugarcane plants may be analysed using any method known in the art for example but not limited to:
The Sampling of Sugar Cane by the Full Width Hatch Sampler; ICUMSA (International Commission for Uniform Methods of Sugar Analysis, http://www.icumsa.org/index.php?id=4) Method GS 5-5 (1994) available from Verlag Dr. Albert Bartens K G, Lückhoffstr. 16, 14129 Berlin (http://www.bartens.com/)
The Sampling of Sugar Cane by the Corer Method; ICUMSA Method GS 5-7 (1994) available from Verlag Dr. Albert Bartens K G, Lückhoffstr. 16, 14129 Berlin (http://www.bartens.com/)
The Determination of Sucrose by Gas Chromatography in Molasses and Factory Products—Official; and Cane Juice; ICUMSA Method G S 4/7/8/5-2 (2002) available from Verlag Dr. Albert Bartens K G, Lückhoffstr. 16, 14129 Berlin (http://www.bartens.com/)
The Determination of Sucrose, Glucose and Fructose by HPLC—in Cane Molasses—and Sucrose in Beet Molasses; ICUMSA Method GS 7/4/8-23 (2011) available from Verlag Dr. Albert Bartens K G, Lückhoffstr. 16, 14129 Berlin (http://www.bartens.com/)
The Determination of Glucose, Fructose and Sucrose in Cane Juices, Syrups and Molasses, and of Sucrose in Beet Molasses by High Performance Ion Chromatography; ICUMSA Method GS 7/8/4-24 (2011) available from Verlag Dr. Albert Bartens K G, Lückhoffstr. 16, 14129 Berlin (http://www.bartens.com/).
   for Crops Other than Sugarcane, Similar Methods are Known in the Art or can Easily be Adapted from a Known Method for Another Crop.

In one embodiment the control plant(s) do not contain an expression cassette of the invention, and hence do not comprise a nucleic acid sequence encoding for a transit peptide and a flavodoxin polypeptide as described herein operably linked to a particular promoter as defined herein.

In another embodiment the control plant(s) carry a nucleic acid sequence encoding for a transit peptide and a flavodoxin polypeptide but this nucleic acid sequence is not functionally linked to the promoter employed in the constructs, vectors, plants, uses and methods of the present invention, i.e. the expression of said nucleic acid sequence is not under the control of said promoter.

Moreover, the present invention relates to the following specific embodiments, wherein the expression “as defined in claim/item X” is meant to direct the artisan to apply the definition as disclosed in item/claim X. For example, “a nucleic acid as defined in item 1” has to be understood such that the definition of the nucleic acid as in item 1 is to be applied to the nucleic acid. In consequence the term “as defined in item” or “as defined in claim” may be replaced with the corresponding definition of that item or claim, respectively:

### Specific Embodiments

1. A method for enhancing one or more yield-related traits in a plant relative to a control plant, comprising increasing the expression in a plant of an exogenous nucleic acid encoding a transit peptide and a flavodoxin polypeptide, wherein the expression is under the control of a promoter sequence operably linked to the nucleic acid encoding the transit peptide and the flavodoxin polypeptide and wherein the promoter sequence comprises the nucleotide sequence of a protochlorophyllide reductase promoter; or functional fragments or derivatives thereof.
2. The method according to embodiment 1, wherein the nucleotide sequence of the protochlorophyllide reductase promoter comprises at least 70% of the sequence represented by SEQ ID NO: 7.
3. The method according to embodiment 1 or 2, wherein the transit peptide targets the flavodoxin polypeptide to a plastid, preferably to a chloroplast.
4. The method according to embodiment 3, wherein the chloroplast transit peptide is selected from the transit peptides listed in Table 4 or homologs thereof.
5. The method according to anyone of embodiments 1 to 4, wherein the flavodoxin polypeptide is encoded by a nucleic acid sequence selected from the group of nucleic acid sequences listed in Table 3 or homologs thereof.
6. The method according to anyone of embodiments 1 to 5, wherein the flavodoxin polypeptide is from Anabaena sp., preferably Anabaena PCC7119.
7. The method according to anyone of embodiments 1 to 6, wherein the flavodoxin polypeptide is encoded by
   (i) an exogenous nucleic acid having at least 60% identity with SEQ ID NO: 1, or a functional fragment thereof, an orthologue or a paralogue thereof; or
   (ii) an exogenous nucleic acid encoding a protein having at least 60% identity with SEQ ID NO: 2, or a functional fragment thereof, an orthologue or a paralogue thereof; or
   (iii) an exogenous nucleic acid capable of hybridizing under stringent conditions with any of the nucleic acids according to (i) or (ii) or a complementary sequence thereof; or
   (iv) an exogenous nucleic acid encoding a polypeptide with the biological activity of a flavodoxin or a ferredoxin; or
   (v) an exogenous nucleic acid encoding the same polypeptide as the nucleic acids of (i) to (iv) above, but differing from the nucleic acids of (i) to (iv) above due to the degeneracy of the genetic code; or
   (vi) an exogenous nucleic acid combining the features of the nucleic acids of any two of (i) to (iv) above.
8. The method according to anyone of embodiments 1 to 7, comprising
   (a) stably transforming a plant cell with an expression cassette comprising an exogenous nucleic acid encoding a transit peptide and encoding a flavodoxin polypeptide, wherein the flavodoxin polypeptide is encoded by
      (i) an exogenous nucleic acid having at least 60% identity with SEQ ID NO: 1, or a functional fragment thereof, an orthologue or a paralogue thereof;
      (ii) an exogenous nucleic acid coding for a protein having at least 60% identity with SEQ ID NO: 2, or a functional fragment thereof, an orthologue or a paralogue thereof; and/or
      (iii) an exogenous nucleic acid capable of hybridizing under stringent conditions with any of the nucleic acids according to (i) or (ii) or a complementary sequence thereof;
      (iv) an exogenous nucleic acid encoding a polypeptide with the biological activity of a flavodoxin or a ferredoxin; or
      (v) an exogenous nucleic acid encoding the same polypeptide as the nucleic acids of (i) to (iv) above, but differing from the nucleic acids of (i) to (iv) above due to the degeneracy of the genetic code; or
      (vi) an exogenous nucleic acid combining the features of the nucleic acids of any two of (i) to (iv) above;
   wherein the exogenous nucleic acid is in functional linkage with a promoter sequence comprising the nucleotide sequence of the protochlorophyllide reductase promoter, or a functional fragment thereof, an orthologue or a paralogue thereof;
   (b) regenerating the plant from the plant cell; and
   (c) expressing said exogenous nucleic acid.
9. Method according to anyone of embodiments 1 to 8, wherein said one or more enhanced yield-related traits comprise enhanced biomass relative to control plants, and preferably comprises enhanced aboveground biomass relative to control plants.
10. Method according to anyone of embodiments 1 to 9, wherein said one or more enhanced yield-related traits are obtained under non-stress conditions or abiotic stress conditions.
11. Method according to embodiment 10, wherein said one or more enhanced yield-related traits are obtained under conditions of drought stress, salt stress, or nitrogen deficiency.
12. Expression construct comprising:
   (i) a nucleic acid encoding a transit peptide as defined in anyone of embodiments 3 or 4 and a flavodoxin polypeptide as defined in anyone of embodiments 5 to 8;
   (ii) a promoter sequences capable of driving expression of the nucleic acid sequence of (i) as defined in embodiment 1 or 2; and optionally
   (iii) a transcription termination sequence.
13. Recombinant expression vector comprising an expression construct according to embodiment 12.
14. Use of an expression construct according to embodiment 12 or a recombinant expression vector according to embodiment 13 in a method for making a transgenic plant having one or more enhanced yield-related traits, preferably increased biomass, relative to control plants, and more preferably increased aboveground biomass relative to control plants.
15. Method for the production of a transgenic plant, transgenic plant part, or transgenic plant cell having one or more enhanced yield-related traits relative to control plants, preferably increased biomass, comprising:
   (a) introducing a recombinant vector construct according to embodiment 13 into a plant, a plant part, or a plant cell;
   (b) generating a transgenic plant, transgenic plant part, or transgenic plant cell from the transformed plant, transformed plant part or transformed plant cell; and
   (c) expressing the exogenous nucleic acid encoding the transit peptide and the flavodoxin polypeptide.
16. The method of embodiment 15, further comprising the step of harvesting propagation material of the transgenic plant and planting the propagation material and growing the propagation material to plants, wherein the propagation material comprises the exogenous nucleic acid encoding the transit peptide and the flavodoxin polypeptide and the promoter sequence operably linked thereto.
17. Transgenic plant, transgenic plant part, or transgenic plant cell obtainable by a method according to any one of embodiments 1 to 11, 15, or 16, wherein said transgenic plant, transgenic plant part, or transgenic plant cell expresses an exogenous nucleic acid encoding a transit peptide and a flavodoxin polypeptide under the control of a promoter sequence as defined in anyone of embodiments 1 to 8.
18. Transgenic plant, transgenic plant part, or transgenic plant cell transformed with an expression construct according to embodiment 12 or with a recombinant expression vector according to embodiment 13, and comprising the promoter sequence operably linked to the nucleic acid encoding the transit peptide and the flavodoxin polypeptide each as defined in any of the embodiments 1 to 8.
19. Transgenic plant, transgenic plant part or transgenic plant cell according to embodiment 17 or 18, wherein the transgenic plant, transgenic plant part or transgenic plant cell has one or more enhanced yield-related traits, preferably an enhanced biomass relative to control plants.
20. Harvestable part of a transgenic plant according to anyone of embodiments 17 to 19, wherein said harvestable part is an above ground organ, preferably the stem or parts thereof.
21. Product produced from a transgenic plant according to anyone of embodiments 17 to 19, or from the harvestable part of a transgenic plant according to embodiment 20.
22. A method for manufacturing a product comprising the steps of growing the transgenic plants according to anyone of embodiments 17 to 19 and producing said product from or by said plants or parts, preferably the stem, of the plant.
23. A method for plant improvement comprising
   a) obtaining a transgenic plant by the method of anyone of embodiments 1 to 11, 15, or 16;
   b) combining within one plant cell the genetic material of at least one plant cell of the plant of a) with the genetic material of at least one cell differing in one or more gene from the plant cells of the plants of a) or crossing the transgenic plant of a) with a second plant;
   c) obtaining seed from at least on plant generated from the one plant cell of b) or the plant of the cross of step (b);
   d) planting said seeds and growing the seeds to plants; and
   e) selecting from said plants, plants expressing the nucleic acid encoding the transit peptide and the flavodoxin polypeptide; and optionally
   f) producing propagation material from the plants expressing the nucleic acid encoding the transit peptide and the flavodoxin polypeptide.
24. The expression construct of embodiment 12 or a recombinant chromosomal DNA comprising an expression cassette comprising a promoter as defined in embodiment 12 item (ii), a nucleic acid encoding a transit peptide linked to a flavodoxin as defined in embodiment 12 item (i) and a transcription termination sequence in functional linkage, wherein the construct or the recombinant chromosomal DNA is comprised in a plant cell.
25. The method according to anyone of embodiments 1 to 11, 15, 16, 22, or 23, or the transgenic plant, transgenic plant part, or transgenic plant cell according to anyone of embodiments 17 to 19, or the use according to embodiment 14, the harvestable part according to embodiment 20, or the product according to embodiment 21, or the construct or recombinant chromosomal DNA of embodiment 24 wherein the plant cell is from or the plant is selected from the group consisting of beans, soya, pea, clover, kudzu, lucerne, lentils, lupins, vetches, groundnut, rice, wheat, maize, barley, arabidopsis, lentil, banana, oilseed rape including canola, cotton, potato, sugar cane, alfalfa, sugar beet, millet, rye, triticale, sorghum, emmer, spelt, einkorn, teff, milo and oats.
26. The method according to anyone of embodiments 1 to 11, 15, 16, 22, or 23, or the transgenic plant, transgenic plant part, or transgenic plant cell according to anyone of embodiments 17 to 19, or the use according to embodiment 14, the harvestable part according to embodiment 20, or the product according to embodiment 21, or the construct or recombinant chromosomal DNA of embodiment 24 wherein the plant cell is from or the plant is a poaceae, preferably of the genus saccharum, more preferably selected from the group consisting of Saccharum arundinaceum, Saccharum bengalense, Saccharum edule, Saccharum munja, Saccharum officinarum, Saccharum procerum, Saccharum ravennae, Saccharum robustum, Saccharum sinense, and Saccharum spontaneum.

### EXAMPLES

The present invention will now be described with reference to the following examples, which are by way of illustration only. The following examples are not intended to limit the scope of the invention.

In particular, the plants used in the described experiments are used because Arabidopsis, tobacco, rice and corn plants are model plants for the testing of transgenes. They are widely used in the art for the relative ease of testing while having a good transferability of the results to other plants used in agriculture, such as but not limited to maize, wheat, rice, soybean, cotton, oilseed rape including canola, sugarcane, sugar beet and alfalfa, or other dicot or monocot crops.

Unless otherwise indicated, the present invention employs conventional techniques and methods of plant biology, molecular biology, bioinformatics and plant breedings.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to SEQ ID NO: 1 and SEQ ID NO: 2

Sequences (full length cDNA, ESTs or genomic) related to SEQ ID NO: 1 and SEQ ID NO: 2 are identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid of SEQ ID NO: 1 is used for the TBLASTN algorithm, with default settings and the filter to ignore low complexity sequences set off. The output of the analysis is viewed by pairwise comparison, and ranked according to the probability score (E-value), where the score reflect the probability that a particular alignment occurs by chance (the lower the E-value, the more significant the hit). In addition to E-values, comparisons are also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

### Example 2

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequencebased searches. The InterPro database combines these databases, which use different methodologies and varying degrees of biological information about well-characterized proteins to derive protein signatures. Collaborating databases include SWISS-PROT, PROSITE, TrEMBL, PRINTS, Propom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom. The results of the InterPro scan (see Zdobnov E. M. and Apweiler R.; “InterProScan—an integration platform for the signature-recognition methods in InterPro.”; Bioinformatics, 2001, 17(9): 847-8; InterPro database, release Release 36.0, 23 Feb. 2012) of the polypeptide sequence as represented by SEQ ID NO: 2 are presented in Table B and FIG. 1.

TABLE B
InterProScan results (major accession numbers) of the
polypeptide sequence as represented by SEQ ID NO: 2.

| | | | Position within |
|---|---|---|---|
| | | | polypeptide |
| Database/ | Accession | Accession | (amino acid |
| method | number | name | residues) |
| PROSITE | PS00201 | FLAVODOXIN | 7-23 |
| PFAM | PF00258 | Flavodoxin_1 | 7-160 |
| PROFILE | PS50902 | FLAVODOXIN_LIKE | 5-165 |
| TIGRFAMs | TIGR01752 | flav_long: flavodoxin | 4-168 |

A repeat analysis using the InterproScan software version 4.8, InterPro database release 41 of Feb. 13, 2013 gave the domains and motifs as listed in table B with the coordinates as given in the last column of table B, and in addition the domains and motifs PIRSF038996, G3DSA:3.40.50.360, PTHR30112, SSF52218 were detected.

In one embodiment a flavodoxin polypeptide comprises a conserved domain (or motif) with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a conserved domain from table B.

### Example 3

### Cloning of the Flavodoxin Encoding Nucleic Acid Sequence

Rice transformation construct:

The nucleic acid encoding transit peptide and flavodoxin polypeptide (SEQ ID NO: 5- or codon optimized for higher plants as shown in SEQ ID NO: 14) or encoding the transit peptide and the Synechocystis flavodoxin (SEQ ID NO: 17) were synthesized so that they include the AttB sites for Gateway recombination (Life Technologies GmbH, Frankfurter Straβe 129B, 64293 Darmstadt, Germany).

Alternatively the nucleic acid sequence coding for the flavodoxin can be amplified by PCR using as template cDNA library in case of eucaryotes or genomic DNA for procaryotes, like Anabaena. PCR is performed using a commercially available proofreading Taq DNA polymerase in standard conditions, using 200 ng of template in a 50 μl PCR mix. The primers used include the AttB sites for Gateway recombination. The amplified PCR fragment is purified also using standard methods.

The first step of the Gateway procedure, the BP reaction, is then performed, during which the PCR fragment recombines in vivo with the pDONR201 plasmid to produce, according to the Gateway terminology, an “entry clone”, pFLD. Plasmid pDONR201 can be purchased from Invitrogen, as part of the Gateway® technology.

A nucleic acid fusing the nucleic acid (SEQ ID NO: 3) for the pea FNR transit peptide to the coding sequence (SEQ ID NO: 2) of the Anabaena flavodoxin may also be generated as described in paragraphs [0075] and [0076] on page 8 of the European patent EP 1 442 127, said paragraphs are incorporated by reference. The resulting nucleic acid sequence may be attached to attB sites to allow for Gateway recombination.

The entry clone comprising the synthesised flavodoxin encoding nucleic acid, of SEQ ID NO: 1, 13 or 15 (linked to the nucleic acid encodingthe transit peptide as shown in SEQ ID NO: 5, 14 and 17, respectively was then used in a LR reaction with a destination vector used for rice transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A PCPR promoter (SEQ ID NO: 7) for specific expression was located upstream of this Gateway cassette. Said promoter was amplified by PCR using genomic DNA of Oryza sativa alternatively it may be synthesized.

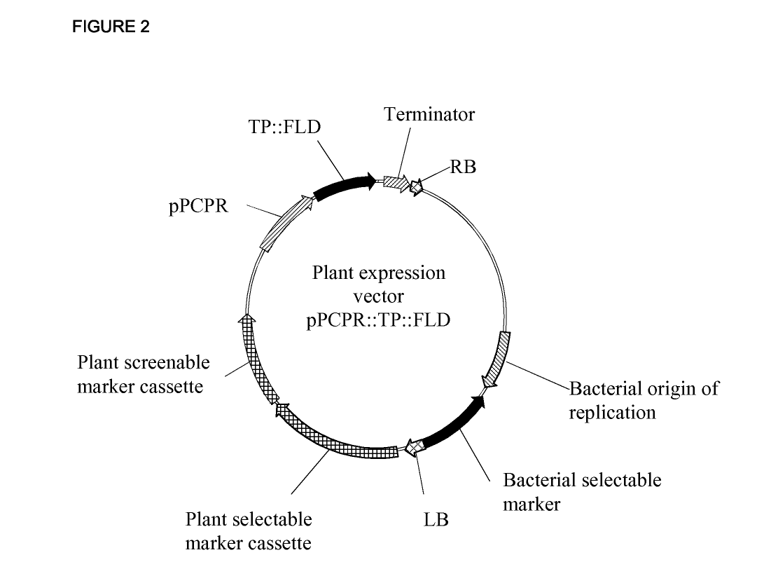

After the LR recombination step, the resulting expression vector PCPR::TP::flavodoxin (FIG. 2) comprising the combination (SEQ ID NO: 19, 20 or 21) of the promoter of SEQ ID NO: 7 with the transit peptide nucleic acid of SEQ ID NO 3 and the flavodoxin nucleic acid (SEQ ID NO: 1, 13 or 15, respectively) was transformed into a suitable Agrobacterium strain according to methods well known in the art.

As an alternative the PCPR promoter (SEQ ID DNO: 7) and the nucleic acid encoding the transit peptide and the Anabaena flavodoxin (SEQ ID NO: 5- or codon optimized for higher plants as shown in SEQ ID NO: 14) or encoding the transit peptide and the Synechocystis flavodoxin (SEQ ID NO: 17) is synthesised as one piece and inserted into a binary vector for Agrobacterium mediated transformation, or in two or more pieces ligated together or assembled to one expression cassette within a vector, e.g. a binary vector.

### Sugarcane Expression Construct

For the expression of the nucleic acid encoding the fusion protein (of transit peptide from Cyanophora paradoxa and flavodoxin from Anabaena) as shown in SEQ ID NO: 11 under control of the PCPR promoter, the PCPR promoter (SEQ ID NO: 7) sequence and the nucleic acid of SEQ ID NO: 9 encoding the transit peptide (SEQ ID NO: 10) and the nucleic acid of SEQ ID NO: 1 coding for the Anabaena flavodoxin of SEQ ID NO: 2 were synthesised linked to the Zein terminator sequence of corn. The resulting expression cassette is shown in SEQ ID NO: 12. To improve the selection efficacy of the transformed plants over non-transformed plants, a selection marker cassette comprising a corn Ubiquitin promoter controlling the expression of the nptII selection marker and the NOS terminator, was included the construct for particle bombardment. Sugarcane plants were transformed with the expression cassette as shown in SEQ ID NO: 12 by particle bombardment. Said expression cassette may be used also for Agrobacterium mediated transformation of sugarcane or other plants after insertion into a binary vector and introduction into Agrobacteria.

The construct comprising the expression cassettes for the transit peptide-flavodoxin expression and the selectable marker cassette may be isolated from the vector as needed and used for particle bombardment of sugarcane cells as described below.

### Example 4

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 to 60 minutes, preferably 30 minutes in sodium hypochlorite solution (depending on the grade of contamination), followed by a 3 to 6 times, preferably 4 time ish with sterile distilled water. The sterile seeds were then germinated on a medium containing 2,4-D (callus induction medium). After incubation in light for 6 days scutellum-derived calli was transformed with Agrobacterium as described herein below.

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The calli were immersed in the suspension for 1 to 15 minutes. The callus tissues were then blotted dry on a filter paper and transferred to solidified, co-cultivation medium and incubated for 3 days in the dark at 25° C. After washing away the Agrobacterium, the calli were grown on 2,4-D-containing medium for 10 to 14 days (growth time for indica: 3 weeks) under light at 28° C.-32° C. in the presence of a selection agent. During this period, rapidly growing resistant callus developed. After transfer of this material to regeneration media, the embryogenic potential was released and shoots developed in the next four to six weeks. Shoots were excised from the calli and incubated for 2 to 3 weeks on an auxin-containing medium from which they were transferred to soil. Hardened shoots were grown under high humidity and short days in a greenhouse.

Transformation of rice cultivar indica can also be done in a similar way as give above according to techniques well known to a skilled person.

35 to 90 independent TO rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

As an alternative, the rice plants may be generated according to the following method: The Agrobacterium containing the expression vector is used to transform Oryza sativa plants. Mature dry seeds of the rice japonica cultivar Nipponbare are dehusked. Sterilization is carried out by incubating for one minute in 70% ethanol, followed by 30 minutes in 0.2% HgCl2, followed by a 6 times 15 minutes wash with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are subcultured on fresh medium 3 days before co-cultivation (to boost cell division activity).

Agrobacterium strain LBA4404 containing the expression vector is used for co-cultivation. Agrobacterium is inoculated on AB medium with the appropriate antibiotics and cultured for 3 days at 28° C. The bacteria are then collected and suspended in liquid co-cultivation medium to a density (OD₆₀₀) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks. Shoots are excised from the calli and incubated for 2 to 3 weeks on an auxin-containing medium from which they are transferred to soil. Hardened shoots are grown under high humidity and short days in a greenhouse.

Approximately 35 to 90 independent TO rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges 1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7 Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Sugarbeet Transformation

Seeds of sugarbeet (Beta vulgaris L.) are sterilized in 70% ethanol for one minute followed by 20 min. shaking in 20% Hypochlorite bleach e.g. Clorox® regular bleach (commercially available from Clorox, 1221 Broadway, Oakland, Calif. 94612, USA). Seeds are rinsed with sterile water and air dried followed by plating onto germinating medium (Murashige and Skoog (MS) based medium (Murashige, T., and Skoog, . . . , 1962. Physiol. Plant, vol. 15, 473-497) including B5 vitamins (Gamborg et al.; Exp. Cell Res., vol. 50, 151-8.) supplemented with 10 g/l sucrose and 0.8% agar). Hypocotyl tissue is used essentially for the initiation of shoot cultures according to Hussey and Hepher (Hussey, G., and Hepher, A., 1978. Annals of Botany, 42, 477-9) and are maintained on MS based medium supplemented with 30 g/l sucrose plus 0.25 mg/l benzylamino purine and 0.75% agar, pH 5.8 at 23-25° C. with a 16-hour photoperiod. Agrobacterium tumefaciens strain carrying a binary plasmid harbouring a selectable marker gene, for example nptII, is used in transformation experiments. One day before transformation, a liquid LB culture including antibiotics is grown on a shaker (28° C., 150 rpm) until an optical density (O.D.) at 600 nm of ˜1 is reached. Overnight-grown bacterial cultures are centrifuged and resuspended in inoculation medium (O.D.˜1) including Acetosyringone, pH 5.5. Shoot base tissue is cut into slices (1.0 cm×1.0 cm×2.0 mm approximately). Tissue is immersed for 30s in liquid bacterial inoculation medium. Excess liquid is removed by filter paper blotting. Co-cultivation occurred for 24-72 hours on MS based medium incl. 30 g/l sucrose followed by a non-selective period including MS based medium, 30 g/l sucrose with 1 mg/l BAP to induce shoot development and cefotaxim for eliminating the Agrobacterium. After 3-10 days explants are transferred to similar selective medium harbouring for example kanamycin or G418 (50-100 mg/l genotype dependent). Tissues are transferred to fresh medium every 2-3 weeks to maintain selection pressure. The very rapid initiation of shoots (after 3-4 days) indicates regeneration of existing meristems rather than organogenesis of newly developed transgenic meristems. Small shoots are transferred after several rounds of subculture to root induction medium containing 5 mg/l NAA and kanamycin or G418. Additional steps are taken to reduce the potential of generating transformed plants that are chimeric (partially transgenic). Tissue samples from regenerated shoots are used for DNA analysis. Other transformation methods for sugarbeet are known in the art, for example those by Linsey & Gallois (Linsey, K., and Gallois, P., 1990. Journal of Experimental Botany; vol. 41, No. 226; 529-36) or the methods published in the international application published as WO9623891A.

### Sugarcane Transformation

Spindles are isolated from 6-month-old field grown sugarcane plants (Arencibia et al., 1998. Transgenic Research, vol. 7, 213-22; Enriquez-Obregon et al., 1998. Planta, vol. 206, 20-27). Material is sterilized by immersion in a 20% Hypochlorite bleach e.g. Clorox® regular bleach (commercially available from Clorox, 1221 Broadway, Oakland, Calif. 94612, USA) for 20 minutes. Transverse sections around 0.5 cm are placed on the medium in the top-up direction. Plant material is cultivated for 4 weeks on MS (Murashige, T., and Skoog, . . . , 1962. Physiol. Plant, vol. 15, 473-497) based medium incl. B5 vitamins (Gamborg, 0., et al., 1968. Exp. Cell Res., vol. 50, 151-8) supplemented with 20 g/l sucrose, 500 mg/l casein hydrolysate, 0.8% agar and 5 mg/l 2,4-D at 23° C. in the dark. Cultures are transferred after 4 weeks onto identical fresh medium. Agrobacterium tumefaciens strain carrying a binary plasmid harbouring a selectable marker gene, for example hpt, is used in transformation experiments. One day before transformation, a liquid LB culture including antibiotics is grown on a shaker (28° C., 150 rpm) until an optical density (O.D.) at 600 nm of ˜0.6 is reached. Overnight-grown bacterial cultures are centrifuged and resuspended in MS based inoculation medium (O.D.˜0.4) including acetosyringone, pH 5.5. Sugarcane embryogenic callus pieces (2-4 mm) are isolated based on morphological characteristics as compact structure and yellow colour and dried for 20 min. in the flow hood followed by immersion in a liquid bacterial inoculation medium for 10-20 minutes. Excess liquid is removed by filter paper blotting. Co-cultivation occurred for 3-5 days in the dark on filter paper which is placed on top of MS based medium incl. B5 vitamins containing 1 mg/l 2,4-D. After co-cultivation calli are washed with sterile water followed by a non-selective cultivation period on similar medium containing 500 mg/l cefotaxime for eliminating remaining Agrobacterium cells. After 3-10 days explants are transferred to MS based selective medium incl. B5 vitamins containing 1 mg/l 2,4-D for another 3 weeks harbouring 25 mg/l of hygromycin (genotype dependent). All treatments are made at 23° C. under dark conditions. Resistant calli are further cultivated on medium lacking 2,4-D including 1 mg/l BA and 25 mg/l hygromycin under 16 h light photoperiod resulting in the development of shoot structures. Shoots are isolated and cultivated on selective rooting medium (MS based including, 20 g/l sucrose, 20 mg/l hygromycin and 500 mg/l cefotaxime). Tissue samples from regenerated shoots are used for DNA analysis. Other transformation methods for sugarcane are known in the art, for example from the international application published as WO2010/151634A and the granted European patent EP1831378.

For transformation by particle bombardment the induction of callus and the transformation of sugarcane can be carried out by the method of Snyman et al. (Snyman et al., 1996, S. Afr. J. Bot 62, 151-154). The construct can be cotransformed with the vector pEmuKN, which expressed the nptII gene (Beck et al. Gene 19, 1982, 327-336; Gen-Bank Accession No. V00618) under the control of the pEmu promoter (Last et al. (1991) Theor. Appl. Genet. 81, 581-588). Plants are regenerated by the method of Snyman et al. 2001 (Acta Horticulturae 560, (2001), 105-108).

### Example 5

### Phenotypic Evaluation Procedure

### Rice Plants

### 5.1 Evaluation Setup

35 to 90 independent TO rice transformants were generated. The primary transformants were transferred from a tissue culture chamber to a greenhouse for growing and harvest of T1 seed. Nine events, of which the T1 progeny segregated 3:1 for presence/absence of the transgene, were retained. For each of these events, approximately six T1 seedlings containing the transgene (hetero- and homo-zygotes) and approximately six T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions were watered at regular intervals to ensure that water and nutrients were not limiting and to satisfy plant needs to complete growth and development, unless they were used in a stress screen.

From the stage of sowing until the stage of maturity the plants were passed several times through a digital imaging cabinet. At each time point digital images (2048×1536 pixels, 16 million colours) were taken of each plant from at least 6 different angles.

T1 events can be further evaluated in the T2 generation following the same evaluation procedure as for the T1 generation, e.g. with less events and/or with more individuals per event.

### Drought Screen

### Early Drought Screen

T1 or T2 plants were germinated under normal conditions and transferred into potting soil as normally. After potting the plants in their pots were then transferred to a “dry” section where irrigation was withheld. Soil moisture probes were inserted in randomly chosen pots to monitor the soil water content (SWC). When SWC went below certain thresholds, the plants were automatically re-watered continuously until a normal level was reached again. The plants were then re-transferred again to normal conditions. The drought cycle was repeated two times during the vegetative stage with the second cycle starting shortly after rewatering after the first drought cycle was complete. The plants were imaged before and after each drought cycle.

The rest of the cultivation (plant maturation, seed harvest) was the same as for plants not grown under abiotic stress conditions. Growth and yield parameters were recorded as detailed for growth under normal conditions.

### Reproductive Drought Screen

T1 or T2 plants were grown in potting soil under normal conditions until they approached the heading stage. They were then transferred to a “dry” section where irrigation was withheld. Soil moisture probes were inserted in randomly chosen pots to monitor the soil water content (SWC). When SWC went below certain thresholds, the plants were automatically re-watered continuously until a normal level was reached again. The plants were then retransferred again to normal conditions. The rest of the cultivation (plant maturation, seed harvest) was the same as for plants not grown under abiotic stress conditions. Growth and yield parameters were recorded as detailed for growth under normal conditions.

### Nitrogen Use Efficiency Screen

T1 or T2 plants were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Salt Stress Screen

T1 or T2 plants are grown on a substrate made of coco fibers and particles of baked clay (Argex) (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Growth and yield parameters are recorded as detailed for growth under normal conditions.

### Sugarcane

5.2.1 The transgenic sugarcane plants generated as described in Example 4 expressing the flavodoxin gene fused to a transit peptide are grown for 10 to 15 months, either in the greenhouse or the field. Standard conditions for growth of the plants are used.

### 5.2.2 Sugar Extraction Method

The extraction of the sugars is done using standard methods for example as described herein above.

### 5.2.3 Fresh Weight and Biomass

Fresh weight and green biomass are measured using a standard method for example as described herein above.

### 5.2.4 Sugar Determination (Glucose, Fructose and Sucrose)

The glucose, fructose and sucrose contents in the extract obtained in accordance with the sugar extraction method described above is determined by one of the standard methods for example as described herein above

### 5.3 Statistical Analysis of Rice Plant Experimental Data: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

### 5.4 Parameters Measured in Rice

From the stage of sowing until the stage of maturity the plants were passed several times through a digital imaging cabinet. At each time point digital images (2048×1536 pixels, 16 million colours) were taken of each plant from at least 6 different angles as described in WO2010/031780. These measurements were used to determine different parameters.

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass (AreaMax).

Increase in root biomass is expressed as an increase in total root biomass (measured as maximum biomass of roots observed during the lifespan of a plant); or as an increase in the root/shoot index, measured as the ratio between root mass and shoot mass in the period of active growth of root and shoot. In other words, the root/shoot index is defined as the ratio of the rapidity of root growth to the rapidity of shoot growth in the period of active growth of root and shoot. Root biomass can be determined using a method as described in WO 2006/029987.

The height of the plant was measured. A robust indication of the height of the plant is the measurement of the location of the centre of gravity, i.e. determining the height (in mm) of the gravity centre of the leafy biomass. This avoids influence by a single erect leaf, based on the asymptote of curve fitting or, if the fit is not satisfactory, based on the absolute maximum.

### Parameters Related to Development Time

Emergence vigour (“EmVg”) is an indication of early plant growth. It is the above-ground biomass of the plant one week after re-potting the established seedlings from their germination trays into their final pots. It is the area (in mm²) covered by leafy biomass in the imaging. It was determined by counting the total number of pixels from aboveground plant parts discriminated from the background. This value was averaged for the pictures taken on the same time point from different angles and was converted to a physical surface value expressed in square mm by calibration.

The “time to flower” of the plant can be determined using the method as described in WO 2007/093444.

The parameter “first panicle” gives the total number of panicles in the first flush.

The parameter “flowers per panicle” is a calculated parameter estimating the average number of florets per panicle on a plant. It is calculated by the number of total seed divided by the first panicle parameter value.

The greenness before flowering is an indication of the greenness of a plant before flowering. It is the proportion (expressed as %) of green and dark green pixels in the last imaging before flowering.

### Seed-Related Parameter Measurements

The mature primary panicles were harvested, counted, bagged, barcode-labelled and then dried for three days in an oven at 37° C. The panicles were then threshed and all the seeds were collected and counted. The seeds are usually covered by a dry outer covering, the husk. The filled husks (herein also named filled florets) were separated from the empty ones using an air-blowing device. The empty husks were discarded and the remaining fraction was counted again. The filled husks were weighed on an analytical balance.

The total number of seeds was determined by counting the number of filled husks that remained after the separation step. The total seed weight was measured by weighing all filled husks harvested from a plant.

The total number of seeds (or florets) per plant was determined by counting the number of husks (whether filled or not) harvested from a plant.

Thousand Kernel Weight (TKW) is extrapolated from the number of seeds counted and their total weight.

The Harvest Index (HI) in the present invention is defined as the ratio between the total seed weight and the above ground area (mm²), multiplied by a factor 106.

The number of flowers per panicle as defined in the present invention is the ratio between the total number of seeds over the number of mature primary panicles.

The “seed fill rate” or “Fillrate” was the proportion (expressed as a %) of the number of filled seeds (i.e. florets containing seeds) over the total number of seeds (i.e. total number of florets). In other words, the seed filling rate is the percentage of florets that are filled with seed.

### Example 6

### Results of the Phenotypic Evaluation of the Transgenic Plants

### 6.1 Rice Plants

### Experiment 1

### Three Flavodoxin Genes Tested Under Standard Conditions and Reproductive Drought Conditions

Using the PCPR promoter of SEQ ID NO: 7 and the nucleic acid of SEQ ID NO: 3 encoding the pea FNR transit peptide, three flavodoxin nucleic acid sequences (SEQ ID NO: 1, 13 and 15) were expressed in the transgenic rice plants generated as described herein above under standard and drought conditions (see example 5). The three nucleic acid sequences were
Nostoc sp. PCC 7119 anabaena sp wildtype flavodoxin sequence (SEQ ID NO: 1),
Synechocystis sp. PCC 6803 wildtype flavodoxin (SEQ ID NO: 15), and
A Nostoc anabaena flavodoxin optimized for plant codon usage (SEQ ID NO: 13).

TABLE Ia
Results for three flavodoxins under standard conditions

| Gene source | TWS | Fillrate | TTF |
|---|---|---|---|
| Nostoc sp. PCC 7119 anabaena sp | −6.4 | 3.9 | 1.8 |
| Synechocystis sp. PCC 6803 | −1.6 | 4.6 | −1.2 |
| codon optimized Nostoc anabaena | 4.2 | 7.0 | −0.8 |
| Average | −1.2 | 5.2 | −0.1 |

TABLE Ib
Results for three flavodoxins under reproductive drought conditions

| Gene source | TWS | Fillrate | TTF |
|---|---|---|---|
| Nostoc sp. PCC 7119 anabaena sp | 4.1 | 19.2 | 1.3 |
| Synechocystis sp. PCC 6803 | 13.5 | 12.2 | 1.6 |
| codon optimized Nostoc anabaena | 11.0 | 5.8 | −0.1 |
| Average | 9.5 | 12.4 | 0.9 |

TWS Total weight of seed;
TTF Time to flower

### Experiment 2

### Nostoc Anabaena Flavodoxin Under Reproductive Drought Conditions

Rice plants carrying the construct comprising the nucleic acid of the Nostoc anabaena flavodoxin (SEQ ID NO: 1) linked to the PCPR promoter of SEQ ID NO: 7 and the nucleic acid of SEQ ID NO: 3 encoding a transit peptide were tested again under reproductive drought conditions and additional parameters were measured.

TABLE II
Results for Nostoc anabaena flavodoxin of SEQ ID NO:
1 under reproductive drought conditions

| Gene source | TWS | Fillrate | TTF | ArMx | HI | EmVg |
|---|---|---|---|---|---|---|
| N. anabaena | −2.45 | 4.72 | 0.82 | 0.81 | 3.11 | 1.56 |

TWS Total weight of seed;
TTF Time to flower;
ArMx AreaMax;
HI Harvest index;
EmVg Emergence vigour

### Experiment 3

### Nostoc Anabaena Flavodoxin Under Standard, Early Drought and Low Nitrogen Conditions

Rice plants carrying the construct comprising the nucleic acid of the Nostoc anabaena flavodoxin (SEQ ID NO: 1) linked to the PCPR promoter of SEQ ID NO: 7 and the nucleic acid of SEQ ID NO: 3 encoding a transit peptide were tested under standard conditions, early drought conditions and low nitrogen conditions. Additional parameters were measured.

TABLE III
Seed yield and biomass yield parameters under three conditions

| | | | | | | |
|---|---|---|---|---|---|---|
| Gene source | TWS | Fillrate | TTF | ArMx | HI | EmVg |

a) standard conditions

| | | | | | | |
|---|---|---|---|---|---|---|
| N. anabaena | 22.92 | 17.29 | 0.81 | −0.72 | 21.43 | −0.83 |

b) early drought

| | | | | | | |
|---|---|---|---|---|---|---|
| N. anabaena | 12.78 | 10.61 | 1.29 | 1.33 | 13.21 | −0.61 |

c) Low nitrogen

| | | | | | | |
|---|---|---|---|---|---|---|
| N. anabaena | 5.19 | 5.86 | −3.01 | 1.08 | 3.47 | −0.49 |

TWS Total weight of seed;
TTF Time to flower;
ArMx AreaMax;
HI Harvest index;
EmVg Emergence vigour

In all experiments, control plants not carrying the construct for overexpression for the flavodoxin were used.

### 6.1.2 Results Summary Over Multiple Experiments

Total weight of the seed:

Table 4 summarizes the seed weight of rice plants expressing the Nostoc anabaena wildtype flavodoxin under control of the PCPR promoter over the different conditions tested.

TABLE IV
Seed weight summary of multiple experiments

| | Reproductive | | | | Early | Low | |
|---|---|---|---|---|---|---|---|
| Gene | drought | | standard | | drought | N | Av- |
| source | Year 1 | Year 2 | Year 1 | Year 3 | Year 3 | Year 3 | erage |
| N. | 4.1 | −2.5 | −6.4 | 22.9 | 12.8 | 5.2 | 6.0 |
| anabaena | | | | | | | |

In addition, the Fillrate and Harvest Index of the rice plants were increased in all experiments.

Under conditions of environmental stress like drought or nitrogen limitation the above ground biomass of the plants was increased as indicated by the AreaMax value of the plants.

Summary of other parameters measured, but not shown in the above tables: The total number of seed and the first panicle value were increased under early drought conditions but decreased under reproductive drought conditions. Flowers per panicle and root to shoot index were increased under early drought conditions but reduced under other conditions. Greenness before flowering, root biomass and thousand kernel weight appeared largely unaffected under all conditions.

### 6.1.3 Summary

The PCPR-transit-peptide-flavodoxin constructs expressed in the transgenic rice plants under different conditions resulted in increased parameters of seed yield as well as of above-ground biomass.

Over all experimental conditions, the total seed weight of rice plants expressing the Nostoc anabaena wildtype flavodoxin under control of the PCPR promoter and linked to a transit peptide as described herein was increased by 6% compared to the one of control plants not carrying this construct. Also, the Fillrate and Harvest Index of the transgenic rice plants were increased under all conditions tested. Under conditions of environmental stress namely drought and nitrogen limitation he above-ground biomass as indicated by the AreaMax value was increased compared to the control plants.

TABLE 2
Examples of flavodoxin nucleic acids as recited in WO 03/035881 on page 35-38

| Accession No | | Gene | Species |
|---|---|---|---|
| NP_358768 | gi\|15903218 | Flavodoxin | Streptococcus pneumoniae R6 |
| NP_345761 | gi\|15901157 | Flavodoxin | Streptococcus pneumoniae TIGR4 |
| NP_311794 | gi\|15833021 | flavodoxin 2 | Escherichia coli 0157:H7] |
| NP_311593 | gi\|15832820 | putative flavodoxin | Escherichia coli 0157:H7 |
| NP_308742 | gi\|15829969 | flavodoxin 1 | Escherichia coli 0157:H7 |
| CAC92877 | gi\|15980620 | flavodoxin 1 | Yersinia pestis |
| CAC89737 | gi\|15978964 | flavodoxin 2 | Yersinia pestis |
| NP_350007 | gi\|15896658 | Flavodoxin | Clostridium acetobutylicum |
| NP_349066 | gi\|15895717 | Flavodoxin | Clostridium acetobutylicum |
| NP_347225 | gi\|15893876 | Flavodoxin | Clostridium acetobutylicum |
| NP_346845 | gi\|15893496 | Flavodoxin | Clostridium acetobutylicum |
| NP_348645 | gi\|15895296 | Predicted flavodoxin | Clostridium acetobutylicum |
| NP_347225 | gi\|15893876 | Flavodoxin | Clostridium acetobutylicum |
| NP_346845 | gi\|15893496 | Flavodoxin | Clostridium acetobutylicum |
| NP_282528 | gi\|15792705 | Flavodoxin | Campylobacter jejuni |
| AAK28628 | gi\|13507531 | Flavodoxin | Aeromonas hydrophila |
| NP_268951 | gi\|15674777 | putative flavodoxin | Streptococcus pyogenes |
| NP_266764 | gi\|15672590 | Flavodoxin | Lactococcus lactis subsp. lactis |
| NP_207952 | gi\|15645775 | flavodoxin (fldA) | Helicobacter pylori 26695 |
| NP_232050 | gi\|15642417 | flavodoxin 2 | Vibrio cholerae |
| NP_231731 | gi\|15642099 | flavodoxin 1 | Vibrio cholerae |
| NP_219360 | gi\|15639910 | Flavodoxin | Treponema pallidum |
| NP_24012 | gi\|15616909 | flavodoxin 1 | Buchnera sp. APS |
| NP_214435 | gi\|15607053 | Flavodoxin | Aquifex aeolicus |
| FXAVEP | gi\|625194 | flavodoxin | Azotobacter vinelandii |
| S38632 | gi\|481443 | flavodoxin | -Synechocystis sp. (strain PCC 6803) |
| FXDV | gi\|476442 | flavodoxin | Desulfovibrio vulgaris |
| A34640 | gi\|97369 | flavodoxin | Desulfovibrio salexigens |
| S24311 | gi\|97368 | flavodoxin | Desulfovibrio gigas (ATCC 19364) |
| A37319 | gi\|95841 | flavodoxin A | Escherichia coli |
| S06648 | gi\|81145 | flavodoxin | red alga (Chondrus crispus) |
| S04600 | gi\|79771 | flavodoxin | Anabaena variabilis |
| A28670 | gi\|79632 | flavodoxin | Synechococcus sp |
| S02511 | gi\|78953 | flavodoxin | Klebsiella pneumoniae |
| FXDVD | gi\|65884 | flavodoxin | Desulfovibrio desulfuricans (ATCC 29577) |
| FXCLEX | gi\|65882 | flavodoxin | Clostridium sp |
| FXME | gi\|65881 | flavodoxin | Megasphaera elsdenii |
| NP_071157 | gi\|11499913 | flavodoxin, putative | Archaeoglobus fulgidus |
| BAA17947 | gi\|1653030 | flavodoxin | Synechocystis sp. PCC 6803 |
| BAB61723 | gi\|14587807 | flavodoxin 2 | Vibrio fischeri |
| BAB61721 | gi\|14587804 | flavodoxin 1 | Vibrio fischeri |
| AAK66769 | gi\|14538018 | flavodoxin | Histophilus ovis |
| P57385 | gi\|11132294 | FLAVODOXIN | |
| AAC7593 | gi\|1789262 | flavodoxin 2 | Escherichia coli K12 |
| AAC73778 | gi\|1786900 | flavodoxin 1 | Escherichia coli K12 |
| AAC75752 | gi\|1789064 | putative flavodoxin | Escherichia coli K12 |
| F69821 | gi\|7429905 | flavodoxin homolog | Bacillus subtilis |
| | | yhcB | |
| QQKBFP | gi\|2144338 | pyruvate | Klebsiella pneumoniae |
| | | (flavodoxin) | |
| | | dehydrogenase nifJ | |
| S16929 | gi\|95027 | flavodoxin A | Azotobacter chroococcum |
| F71263 | gi\|7430914 | probable flavodoxin | Syphilis spirochete |
| A64665 | gi\|7430911 | flavodoxin | Helicobacter pylori (strain 26695 |
| JE0109 | gi\|7430907 | flavodoxin | Desulfovibrio vulgaris |
| S42570 | gi\|628879 | flavodoxin | Desulfovibrio desulfuricans (ATCC 27774) |
| BAB13365 | gi\|10047146 | flavodoxin | Alteromonas sp. O-7 |
| AAF34250 | gi\|6978032 | flavodoxin | Desulfovibrio gigas |
| CAB73809 | gi\|6968816 | flavodoxin | Campylobacter jejuni |
| D69541 | gi\|7483302 | flavodoxin homolog | Archaeoglobus fulgidus |
| F70479 | gi\|7445354 | flavodoxin | Aquifex aeolicus |
| S55234 | gi\|1084290 | flavodoxin isoform I | Chlorella fusca |
| S18374 | gi\|2117434 | flavodoxin | Anabaena sp. (PCC 7119) |
| S55235 | gi\|1084291 | flavodoxin isoform II | Chlorella fusca |
| C64053 | gi\|1074088 | flavodoxin A | Haemophilus influenzae (strain Rd KW20) |
| A61338 | gi\|625362 | flavodoxin | Clostridium pasteurianum |
| A39414 | gi\|95560 | flavodoxin | Enterobacter agglomerans plasmid pEA3 |
| AAD08207 | gi\|2314319 | flavodoxin (fldA) | Helicobacter pylori 26695 |
| CAB37851 | gi\|4467982 | flavodoxin | Rhodobacter capsulatus |
| AAC65882 | gi\|3323245 | flavodoxin | Treponema pallidum |
| AAB88920 | gi\|2648181 | flavodoxin, putative | Archaeoglobus fulgidus |
| AAB65080 | gi\|2289915 | flavodoxin | Klebsiella pneumoniae |
| AAB53659 | gi\|710356 | flavoprotein | Methanothermobacter thermautotrophicus |
| AAB51076 | gi\|1914879 | flavodoxin | Klebsiella pneumoniae |
| AAB36613 | gi\|398014 | flavodoxin | Azotobacter chroococcum |
| AAB20462 | gi\|239748 | flavodoxin | Anabaena |
| AAA64735 | gi\|142370 | flavodoxin (nifF) | Azotobacter vinelandii |
| BAA35341 | gi\|1651296 | Flavodoxin | Escherichia coli |
| BAA35333 | gi\|1651291 | Flavodoxin | Escherichia coli |
| AAA27288 | gi\|415254 | flavodoxin | Synechocystis sp. |
| AAA27318 | gi\|154528 | Flavodoxin | Synechococcus sp. |
| AAC45773 | gi\|1916334 | putative flavodoxin | Salmonella typhimurium |
| AAC07825 | gi\|2984302 | flavodoxin | Aquifex aeolicus |
| AAC02683 | gi\|2865512 | flavodoxin | Trichodesmium erythraeum |

TABLE 3
Examples of chloroplast transit peptides as recited in WO 03/035881 on page 39-45

| Accession No | | Gene | Species |
|---|---|---|---|
| P32260 | gi\|12644209 | CYSTEINE SYNTHASE, CHLOROPLAST | Spinacia oleracea |
| | | PRECURSOR | |
| AAG59996 | gi\|12658639 | ferredoxin:sulfite reductase precursor | Glycine max |
| S10200 | gi\|100078 | carbonate dehydratase precursor | Pisum sativum |
| CAB89287 | gi\|7672161 | chloroplast FtsZ-like protein | Nicotiana tabacum |
| P17067 | gi\|115471 | CARBONIC ANHYDRASE, CHLOROPLAST | Pisum sativum |
| | | PRECURSOR (CARBONATE DEHYDRATASE) | |
| AAD22109 | gi\|4530595 | heme oxygenase 2 | Arabidopsis thaliana |
| AAD22108 | gi\|4530593 | heme oxygenase 1 | Arabidopsis thaliana |
| AAC50035 | gi\|450235 | APS kinase | Arabidopsis thaliana |
| AAC12846 | gi\|1051180 | phytoene desaturase | Zea mays |
| AAB87573 | gi\|2645999 | chlorophyll a/b binding protein of LHCII type I precursor | Panax ginseng |
| CAA47329 | gi\|312944 | cysteine synthase | Spinacia oleracea |
| CAA31137 | gi\|41201 | O-acetylserine sulfhydrylase | Escherichia coli |
| AAA82068 | gi\|1079732 | cpFtsZ | Arabidopis thaliana |
| T06368 | gi\|7489040 | photosystem II oxygen-evolving complex protein 1 precursor | Lycopersicon esculentum |
| S71750 | gi\|7488813 | import intermediate-associated 100K protein precursor | Pisum sativum |
| S71749 | gi\|7459239 | DCL protein precursor, chloroplast | Lycopersicon esculentum |
| 15825883 | gi\|15825883 | Chain B, Structure of Threonine Synthase | Arabidopsis thaliana |
| 15825882 | gi\|15825882 | Chain A, Structure Of Threonine Synthase | Arabidopsis thaliana |
| T09543 | gi\|7488970 | deoxyxylulose synthase TKT2 precursor | Capsicum annum |
| JC5876 | gi\|7447856 | early light-inducible protein precursor | Glycine max |
| P24493 | gi\|1170215 | DELTA-AMINOLEVULINIC ACID DEHYDRATASE | Spinacia oleracea |
| | | PRECURSOR | |
| S47966 | gi\|1076532 | probable lipid transfer protein M30 precursor | Pisum sativum |
| A44121 | gi\|322404 | ribosomal protein S1 precursor | Spinacia oleracea |
| S01056 | gi\|81896 | early light-induced protein precursor | Pisum sativum |
| O22773 | gi\|7388292 | THYLAKOID LUMENAL 16.5 KDA PROTEIN, | Arabidopsis |
| | | CHLOROPLAST PRECURSOR | thaliana |
| P80470 | gi\|6093830 | PHOTOSYSTEM II CORE COMPLEX PROTEINS PSBY | Spinacia oleracea |
| | | PRECURSOR | |
| P55195 | gi\|1709930 | PHOSPHORIBOSYLAMINOMIDAZOLE | Vigna aconitifolia |
| | | CARBOXYLASE, CHLOROPLAST PRECURSOR | |
| P11970 | gi\|1709654 | PLASTOCYANIN B, CHLOROPLAST PRECURSOR | Populus nigra |
| P00299 | gi\|1709651 | PLASTOCYANIN A, CHLOROPLAST PRECURSOR | Populus nigra |
| P80484 | gi\|1709608 | PERIDININ-CHLOROPHYLL A PROTEIN 1 PRECURSOR | Amphidinium carterae |
| P08823 | gi\|134102 | RUBISCO SUBUNIT BINDING-PROTEIN ALPHA | Triticum aestivum |
| | | SUBUNIT PRECURSOR | |
| P04045 | gi\|130173 | ALPHA-1,4 GLUCAN PHOSPHORYLASE, L-1 | Solanum tuberosum |
| | | ISOZYME, CHLOROPLAST PRECURSOR | |
| S30897 | gi\|7427677 | 3-isopropylmalate dehydrogenese precursor | Solanum tuberosum |
| TXSPM | gi\|7427615 | thioredoxin m precursor | Spinacia oleracea |
| FEKM | gi\|7427604 | ferredoxin [2Fe—2S] precursor | Chlamydomonas |
| | | | reinhardtii |
| CCKM6R | gi\|2144284 | cytochrome c6 precursor | Chlamydomonas |
| | | | reinhardtii |
| S30145 | gi\|419757 | ketol-acid reductoisomerase precursor | Arabidopisis thaliana |
| DEMZMC | gi\|319840 | malate dehydrogenase (NADP+) precursor | Zea mays |
| S20510 | gi\|81676 | 3-isopropylmalate dehydrogenase precursor | Brassica napus |
| S17180 | gi\|81509 | ketol-acid reductoisomerase precursor | Spinacia oleracea |
| Q9SEL7 | gi\|15214049 | PROTEASE HHOA, CHLOROPLAST PRECURSOR | Arabidopsis thaliana |
| O23403 | gi\|13959580 | THYLAKOID LUMENAL 21.5 KDA PROTEIN, | Arabidopsis thaliana |
| | | CHLOROPLAST PRECURSOR | |
| P82281 | gi\|12644689 | PUTATIVE L-ASCORBATE PEROXIDASE, | Arabidopsis thaliana |
| | | CHLOROPLAST PRECURSOR | |
| O22609 | gi\|9910645 | PROTEASE DO-LIKE, CHLOROPLAST PRECURSOR | Arabidopsis thaliana |
| P48417 | gi\|1352186 | ALLENE OXIDE SYNTHASE, CHLOROPLAST | Linum usitatissimum |
| | | PRECURSOR | |
| P49080 | gi\|1351905 | BIFUNCTIONAL ASPARTOKINASE/HOMOSERINE | Zea mays |
| | | DEHYDROGENASE 2, CHLOROPLAST PRECURSOR | |
| P31853 | gi\|461595 | ATP SYNTHASE B′ CHAIN, CHLOROPLAST | Spinacia oleracea |
| | | PRECURSOR | |
| P10933 | gi\|119905 | FERREDOXIN--NADP REDUCTASE, LEAP ISOZYME | Pisum sativum |
| | | PRECURSOR | |
| P52422 | gi\|14917033 | PHOSPHORIBOSYLGLYCINAMIDE FORMYL | Arabidopsis thaliana |
| | | TRANSFERASE, CHLOROPLAST PRECURSOR | |
| p49077 | gi\|14917032 | ASPARTATE CARBAMOYLTRANSFERASE | Arabidopsis thaliana |
| | | PERCURSOR | |
| O50039 | gi\|14917022 | ORNITHINE CARBAMOYLTRANSFERASE | Arabidopsis thaliana |
| | | CHLOROPLAST PRECURSOR | |
| P55229 | gi\|14916987 | GLUCOSE-1-PHOSPHATE ADENYLYLTRANSFERASE | Arabidopsis thaliana |
| | | LARGE SUBUNIT 1, CHLOROPLAST PRECURSOR | |
| Q96291 | gi\|14916972 | 2-CYS PEROXIREDOXIN BAS1, CHLOROPLAST | Arabidopsis thaliana |
| | | PRECURSOR | |
| Q9ZT00 | gi\|14916690 | RIBULOSE BISPHOSPHATE CARBOXYLASE/ | Zea mays |
| | | OXYGENASE ACTIVASE, CHLOROPLAST PRECURSOR | |
| Q9LZX6 | gi\|14547977 | DIHYDRODIPICOLINATE SYNTHASE 1, | Arabidopsis thaliana |
| | | CHLOROPLAST PRECURSOR | |
| O64903 | gi\|12644076 | NUCLEOSIDE DIPHOSPHATE KINASE II, | Arabidopsis thaliana |
| | | CHLOROPLAST PRECURSOR | |
| O04130 | gi\|3122858 | D-3-PHOSPHOGLYCERATE | Arabidopsis thaliana |
| | | DEHYDROGENASE PRECURSOR | |
| O24364 | gi\|3121825 | 2-CYC PEROXIREDOXIN BAS1, CHLOROPLAST | Spinacia oleracea |
| | | PRECURSOR | |
| P49107 | gi\|1709825 | PHOTOSYSTEM I REACTION CENTRE SUBUNIT | Arabidopsis thaliana |
| | | N PRECURSOR | |
| P49132 | gi\|1352199 | TRIOSE PHOSPHATE/PHOSPHATE | Flaveria trinervia |
| | | TRANSLOCATOR, CHLOROPLAST PRECURSOR | |
| P37107 | gi\|586038 | SIGNAL RECOGNITION PARTICLE 54 KDA | Arabidopsis thaliana |
| | | PROTEIN, CHLOROPLAST PRECURSOR | |
| Q04836 | gi\|464662 | 31 KDA RIBONUCLEOPROTEIN, CHLOROPLAST | Arabidopsis thaliana |
| | | PRECURSOR | |
| Q01909 | gi\|461551 | ATP SYNTHASE GAMMA CHAIN 2, CHLOROPLAST | Arabidopsis thaliana |
| | | PRECURSOR | |
| P14671 | gi\|136251 | TRYPTOPHAN SYNTHASE BETA CHAIN 1 | Arabidopsis thaliana |
| | | PRECURSOR | |
| P07089 | gi\|132144 | RIBULOSE BISPHOSPHATE CARBOXYLASE SMALL | Flaveria trinervia |
| | | CHAIN PRECURSOR | |
| P22221 | gi\|130384 | PYRUVATE, PHOSPHATE DIKINASE PRECURSOR | Flaveria trinervia |
| P22178 | gi\|126736 | NADP-DEPENDENT MALIC ENZYME, CHLOROPLAST | Flaveria trinervia |
| | | PRECURSOR | |
| P26259 | gi\|118241 | DIHYDRODIPICOLINATE SYNTHASE, CHLOROPLAST | Zea mays |
| | | PRECURSOR | |
| P23577 | gi\|118044 | APOCYTOCHROME F PRECURSOR | Chlamydomonas |
| | | | reinhardtii |
| Q42522 | gi\|14195661 | GLUTAMATE-1-SEMIALDEHYDE 2,1-AMINOMUTASE | Arabidopsis thaliana |
| | | 2 PRECURSOR | |
| Q96242 | gi\|13878924 | ALLENE OXIDE SYNTHASE PRECURSOR | Arabidopsis thaliana |
| P46312 | gi\|13432148 | OMEGA-6 FATTY ACID DESATURASE, CHLOROPLAST | Arabidopsis thaliana |
| | | PRECURSOR | |
| P34802 | gi\|13432144 | GERANYLGERANYL PYROPHOSPHATE | Arabidopsis thaliana |
| | | SYNTHETASE, CHLOROPLAST PRECURSOR | |
| P50318 | gi\|12644295 | PHOSPHOGLYCERATE KINASE, CHLOROPLAST | Arabidopsis thaliana |
| | | PERCURSOR | |
| P46309 | gi\|12644273 | GLUTAMATE--CYSTEINE LIGASE, CHLOROPLAST | Arabidopsis thaliana |
| | | PRECURSOR | |
| P21276 | gi\|12644157 | SUPEROXIDE DISMUTASE [FE], CHLOROPLAST | Arabidopsis thaliana |
| | | PERCURSOR | |
| O23787 | gi\|6094476 | THIAZOLE BIOSYNTHETIC ENZYME, CHLOROPLAST | Citrus sinensis |
| | | PERCURSOR | |
| P93407 | gi\|3915008 | SUPEROXIDE DISMUTASE [CU-ZN], CHLOROPLAST | Oryza sativa |
| | | PRECURSOR | |
| Q96255 | gi\|3914995 | PHOSPHERINE AMINOTRANSFERASE, CHLOROPLAST | Arabidopsis thaliana |
| | | PRECURSOR | |
| O24600 | gi\|3914826 | DNA-DIRECTED RNA POLYMERASE, CHLOROPLAST | Arabidopsis thaliana |
| | | PERCURSOR | |
| O49937 | gi\|3914665 | 50S RIBOSOMAL PROTEIN L4, CHLOROPLAST | Spinacia oleracea |
| | | PRECURSOR | |
| Q42915 | gi\|3914508 | RIBULOSE BISPHOSPHATE CARBOXYLASE | Manihot esculenta |
| | | SMALL CHAIN PRECURSOR | |
| Q39199 | gi\|2500098 | DNA REPAIR PROTEIN RECA, CHLOROPLAST | Arabidopsis thaliana |
| | | PRECURSOR | |
| Q96529 | gi\|2500026 | ADENYLOSUCCINATE SYNTHETASE PRECURSOR | Arabidopsis thaliana |
| P55826 | gi\|2495184 | PROTOPORPHYRINOGEN OXIDASE, | Arabidopsis thaliana |
| | | CHLOROPLAST PRECURSOR | |
| Q42496 | gi\|2493687 | CYTOCHROME B6-F COMPLEX 4 KDA SUBUNIT, | Chlamydomonas |
| | | CHLOROPLAST PRECURSOR | reinhardtii |
| P52424 | gi\|1709925 | PHOSPHORIBOSYLFORMYLGLYCINAMIDINE | Vigna unguiculata |
| | | CYCLO-LIGASE, CHLOROPLAST PRECURSOR | |
| P49572 | gi\|1351303 | INDOLE-3-GLYCEROL PHOSPHATE SYNTHASE, | Arabidopsis thaliana |
| | | CHLOROPLAST PRECURSOR | |
| P48496 | gi\|1351271 | TRIOSEPHOSPHATE ISOMERASE, CHLOROPLAST | Spinacia oleracea |
| | | PRECURSOR | |
| P25269 | gi\|1174779 | TRYPTOPHAN SYNTHASE BETA CHAIN 2 | Arabidopsis thaliana |
| | | PRECURSOR | |
| P46225 | gi\|1174745 | TRIOSEPHOSPHATE ISOMERASE, CHLOROPLAST | Secale cereale |
| | | PRECURSOR | |
| P46283 | gi\|1173345 | SEDOHEPTULOSE-1-7-BISPHOSPHATASE, | Arabidopsis thaliana |
| | | CHLOROPLAST PRECURSOR | |
| P32069 | gi\|418134 | ANTHRANILATE SYNTHASE COMPONENT I-2 | Arabidopsis thaliana |
| | | PRECURSOR | |
| P29450 | gi\|267120 | THIOREDOXIN F-TYPE, CHLOROPLAST | Pisum sativum |
| | | PRECURSOR | |
| Q9ZTN9 | gi\|13878459 | PHYTOENE DEHYDROGENASE PRECURSOR | Oryza sativa |
| Q9SHI1 | gi\|13627881 | TRANSLATION INTIATION FACTOR IF-2, | Arabidopsis thaliana |
| | | CHLOROPLAST PRECURSOR | |
| Q9LR75 | gi\|13431553 | COPROPORPHYRINOGEN III OXIDASE, | Arabidopsis thaliana |
| | | CHLOROPLAST PRECURSOR | |
| Q9ZNZ7 | gi\|12643970 | FERREDOXIN-DEPENDENT GLUTAMATE | Arabidopsis thaliana |
| | | SYNTHASE 1, CHLOROPLAST PRECURSOR | |
| Q9SZ30 | gi\|12643854 | BIFUNCTIONAL HISTIDINE BIOSYNTHESIS | Arabidopsis thaliana |
| | | PROTEIN HISHF, CHLOROPLAST PRECURSOR | |
| Q9SJE1 | gi\|12643848 | MAGNESIUM-CHELATASE SUBUNIT CHLD | Arabidopsis thaliana |
| | | PRECURSOR | |
| Q42624 | gi\|12643761 | GLUTAMINE SYNTHETASE, CHLOROPLAST | Brassica napus |
| | | PRECURSOR | |
| Q38933 | gi\|12643749 | LYCOPENE BETA CYCLASE CHLOROPLAST | Arabidopsis thaliana |
| | | PRECURSOR | |
| Q42435 | gi\|12643508 | CAPSANTHIN/CAPSORUBIN SYNTHASE, | Capsicum annuum |
| | | CHLOROPLAST PRECURSOR | |

1-25. (canceled)

26. A method for enhancing above ground biomass or seed yield or storage carbohydrate content in a crop plant species, the method comprising the steps of:
a) stably transforming a cell of said crop plant with an expression cassette comprising a polynucleotide encoding a protochlorophyllide reductase promoter in operative association with a polynucleotide encoding a transit peptide and a polynucleotide encoding a flavodoxin polypeptide;
b) regenerating a population of transformed plants from said transformed cell; and
c) selecting from said population transformed plants expressing the flavodoxin polypeptide and exhibiting increased above ground biomass or seed yield, as compared to crop plants of the same species which do not express the flavodoxin polypeptide.

27. The method of claim 26, wherein the polynucleotide encoding the protochlorophyllide reductase promoter comprises the sequence set forth in SEQ ID NO: 7.

28. The method of claim 26, wherein the flavodoxin polypeptide has a sequence selected from the group consisting of SEQ ID NO: 2 and SEQ ID NO: 16.

29. The method of claim 26, wherein the expression cassette comprises a promoter having the sequence set forth in SEQ ID NO: 7, and a polynucleotide having a sequence selected from the group consisting of SEQ ID NO: 5, SEQ ID NO: 8, SEQ ID NO: 14 and SEQ ID NO: 17.

30. The method of claim 26, wherein the crop plant species is selected from the group consisting of rice, wheat, maize, cotton, sugar cane, alfalfa and sugar beet.

31. The method of claim 30, wherein the crop plant is maize.

32. The method of claim 30, wherein the crop plant is rice.

33. The method of claim 30, wherein the crop plant is sugar cane.

34. A method for enhancing above ground biomass or seed yield or storage carbohydrate content in a crop plant species, the method comprising the steps of:
a) stably transforming a cell of said crop plant with an expression cassette comprising a polynucleotide encoding a protochlorophyllide reductase promoter in operative association with a polynucleotide encoding a transit peptide and a polynucleotide encoding a flavodoxin polypeptide comprising the PFAM domain PF00258;
b) regenerating a population of transformed plants from said transformed cell; and
c) selecting from said population transformed plants expressing the flavodoxin polypeptide and exhibiting increased above ground biomass or seed yield, as compared to crop plants of the same species which do not express the flavodoxin polypeptide.

35. The method of claim 34, wherein the polynucleotide encoding the protochlorophyllide reductase promoter comprises the sequence set forth in SEQ ID NO: 7.

36. The method of claim 34, wherein the flavodoxin polypeptide has a sequence selected from the group consisting of SEQ ID NO: 2 and SEQ ID NO: 16.

37. The method of claim 34, wherein the expression cassette comprises a promoter having the sequence set forth in SEQ ID NO: 7, and a polynucleotide selected from the group consisting of SEQ ID NO: 5, SEQ ID NO: 8, SEQ ID NO: 14 and SEQ ID NO: 17.

38. The method of claim 34, wherein the crop plant species is selected from the group consisting of rice, wheat, maize, cotton, sugar cane, alfalfa and sugar beet.

39. The method of claim 34, wherein the crop plant is maize.

40. The method of claim 34, wherein the crop plant is rice.

41. The method of claim 34, wherein the crop plant is sugar cane.

42. The method of claim 41 wherein the enhanced above ground biomass is stem, stalk and/or sett biomass, or parts thereof.

43. A method for manufacturing a product comprising the steps of:
a) stably transforming a cell of a Poaceae plant with an expression cassette comprising a polynucleotide encoding a protochlorophyllide reductase promoter in operative association with a polynucleotide encoding a transit peptide and a polynucleotide encoding a flavodoxin polypeptide;
b) regenerating a population of transformed plants from said transformed cell;
c) selecting from said population transformed plants expressing the flavodoxin polypeptide and exhibiting increased above ground biomass, as compared to crop plants of the same species which do not express the flavodoxin polypeptide;
d) growing said selected Poaceae plants;
e) obtaining the stem from said plants or parts thereof, and
f) extracting the juice from the stem and/or extracting the residual fibers after juice extraction, and optionally
g) extracting sugar from the juice of the stem.

44. The method of claim 43 wherein the Poaceae plant is a Saccharum species.

45. The method of claim 43 wherein the Poaceae plant is a sugarcane plant.
